(19) United States
(12) Patent Application Publication
(10) Pub. No.: US 2023/0138851 A1
(43) Pub. Date: May 4, 2023

(54) NOVEL ALKYNE DERIVATIVES

(71) Applicant: CARNA BIOSCIENCES, INC., Hyogo (JP)

(72) Inventors: Ayako SAWA, Osaka-shi, Osaka (JP); Wataru KAWAHATA, Kobe-shi, Hyogo (JP); Yuko ASAMITSU, Kobe-shi, Hyogo (JP); Masaaki SAWA, Ibaraki-shi, Osaka (JP); Yasuhiro IWATA, Taito-ku, Tokyo (JP); Hideki MORIYAMA, Kobe-shi, Hyogo (JP); Shingo TOJO, Takarazuka-shi, Hyogo (JP); Daisuke URABE, Osaka-shi, Osaka (JP)

(73) Assignee: CARNA BIOSCIENCES, INC., Hyogo (JP)

(21) Appl. No.: 17/795,728

(22) PCT Filed: Jan. 28, 2021
(86) PCT No.: PCT/JP2021/003026

§ 371 Date: Jul. 27, 2022

(30) Foreign Application Priority Data
Jan. 30, 2020 (JP) 2020-013536

(51) Int. Cl. A61K 31/429; C07D 513/04; A61K 31/437; A61K 31/5365

(52) U.S. Cl. CPC A61K 31/429; C07D 513/04; A61K 31/437; A61K 31/5365; C07B 2200/05

(57) ABSTRACT

The present invention provides a compound that has an inhibitory effect on DYRK and that is represented by general formula (I):

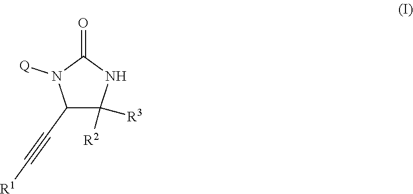

wherein Q, R¹, R² and R³ are as defined in the description.

The present invention relates to a medicament, particularly a novel alkyne derivative having a DYRK inhibitory effect or a pharmaceutically acceptable salt thereof.

### BACKGROUND ART

DYRK (dual-specificity tyrosine-phosphorylation regulated kinase) is one of the bispecific protein kinases that phosphorylate tyrosine, serine, and threonine. DYRK functions as a tyrosine kinase only in the case of autophosphorylation and catalyzes the phosphorylation of serine or threonine residues on exogenous substrates. Five members of the DYRK family are known in humans: DYRK1A, DYRK1B, DYRK2, DYRK3, and DYRK4 (Non Patent Literature 1).

It has been widely reported that DYRK1A is associated with neuropsychiatric diseases. For example, in patients with Alzheimer's disease, the expression of β-amyloid is significantly consistent with that of DYRK1A (Non Patent Literature 2), and it is speculated that DYRK1A is involved in abnormal phosphorylation of a tau protein (Tau), which is considered to contribute to the onset of Alzheimer's disease (Non Patent Literature 3).

In addition, Parkinson's disease is a neurodegenerative disease caused by the degeneration of dopamine neurons, which are important for motor function, but one of the causes is considered to be mitochondrial dysfunction (Non Patent Literature 4). An enzyme involved in protein degradation called Parkin is known to metabolize abnormal mitochondria and suppress abnormal accumulation, but DYRK1A has been reported to suppress the activity of this parkin protein (Non Patent Literature 5).

The gene for DYRK1A is located in the Down's syndrome critical region, and it has been reported that mice overexpressing DYRK1A exhibit neuropsychiatric dysfunction and appear like Down's syndrome (Non Patent Literature 6). It has also been reported that DYRK1A expression is increased in the brain of patients with Down's syndrome and Down's syndrome-like model mice (Non Patent Literature 7). These reports suggest that DYRK1A is involved in the onset of neurological symptoms in the patients with Down's syndrome (Non Patent Literature 8).

In addition, it has been reported that early-onset Alzheimer's disease occurs frequently in patients with Down's syndrome, thus indicating that DYRK1A is closely related to Alzheimer's disease (Non Patent Literature 8).

Therefore, compounds inhibiting DYRK1A are considered useful for treating neuropsychiatric diseases such as Alzheimer's disease, Down's syndrome, mental retardation, memory impairment, memory loss, and Parkinson's disease.

Recently, it has been reported that DYRK1A is highly expressed in brain tumors such as glioblastoma and regulates the expression of an epidermal growth factor receptor (EGFR) (Non Patent Literature 9). Therefore, compounds inhibiting DYRK1A are considered useful for treating EGFR-dependent cancers by suppressing the proliferation of cancer cells in EGFR-dependent brain tumors and other tumors.

Compounds inhibiting the family enzymes DYRK1B, DYRK2, and DYRK3 are also considered to have various pharmaceutical applications. For example, it has been reported that DYRK1B is highly expressed in quiescent (GO-phase) cancer cells and contributes to resistance to various chemotherapeutic agents (Non Patent Literature 10). It has also been reported that inhibition of DYRK1B promotes withdrawal from the GO phase and enhances sensitivity to chemotherapeutic agents (Non Patent Literature 11). Therefore, compounds inhibiting DYRK1B are considered useful for treating pancreatic cancer, ovarian cancer, osteosarcoma, colorectal cancer, and lung cancer (Non Patent Literatures 11, 12, 13, 14, and 15).

It is suggested that DYRK2 controls p53 to induce apoptosis in response to DNA damages (Non Patent Literature 16). Furthermore, it has been reported that compounds inhibiting DYRK3 are useful for treating sickle cell anemia and chronic kidney disease (Non Patent Literature 17).

In addition to Patent Literature 1 for compounds inhibiting DYRK, Patent Literature 2 has been reported for DYRK1A and DYRK1B inhibitors. However, the alkyne derivative of the present invention is not disclosed therein.

### PRIOR ART DOCUMENT(S)

### Patent Document(s)

[Patent Literature 1] WO2010/10797
[Patent Literature 2] WO2013/26806

### Non-Patent Document(s)

[Non-Patent Literature 1] Becker W. et al., J. Biol. Chem., 1998, 273, 25893-25902
[Non-Patent Literature 2] Kimura R. et al., Hum. Mol. Genet., 2007, 16, 15-23
[Non-Patent Literature 3] Ryoo S R. et al., J. Biol. Chem., 2007, 282, 34850-34857
[Non-Patent Literature 4] Narendra D. et al., J. Cell. Biol., 2008, 183, 795-803
[Non-Patent Literature 5] Im E., J. Neurochem., 2015,134, 756-768
[Non-Patent Literature 6] Branchi I. et al., J. Neuropathol. Exp. Neurol., 2004, 63, 429-440
[Non-Patent Literature 7] Dowjat W K. et al., Neurosci. Lett., 2007, 413, 77-81
[Non-Patent Literature 8] Wegiel J. et al., FEBS J., 2011,278, 236-245
[Non-Patent Literature 9] Pozo N. et al., J. Clin. Invest., 2013, 123, 2475-2487.
[Non-Patent Literature 10] Deng X. et al., Cancer Res., 2006, 66, 4149-4158.
[Non-Patent Literature 11] Ewton D Z. et al., Mol. Cancer Ther., 2011, 10, 2104-2114.
[Non-Patent Literature 12] Deng X. et al., Genes Cancer., 2014, 5, 201-211.
[Non-Patent Literature 13] Yang C. et al., Carcinogenesis., 2010, 31, 522-528.
[Non-Patent Literature 14] Jin K. et al., J. Biol. Chem., 2009,
[Non-Patent Literature 15] Gao J et al., Cancer Cell Int. 2013, 13, 2
[Non-Patent Literature 16] Taira N. et al., Mol. Cell., 2007, 25, 725-738.
[Non-Patent Literature 17] Bogacheva O. et al., J. Biol. Chem., 2008, 283, 36665-36675.

### DISCLOSURE OF INVENTION

### Problem to be Solved by the Invention

An object of the present invention is to provide a medicament, particularly a novel compound having a DYRK inhibitory effect.

### Means for Solving Problem

The object of the present invention is achieved by the following (1) to (18).

(1) An alkyne derivative of the following formula (I):

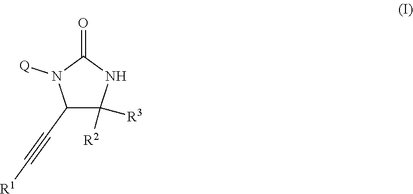

wherein R¹ represents a hydrogen atom, a halogen atom, a trimethylsilyl group, an optionally substituted aryl group, an optionally substituted heteroaryl group, an optionally substituted lower alkyl group, and an optionally substituted cycloalkyl group;

R² and R³ represent each independently a hydrogen atom, a halogen atom, an optionally substituted lower alkyl group, an optionally substituted cycloalkyl group, an optionally substituted aryl group, an optionally substituted heteroaryl group, an optionally substituted saturated heterocyclic group, an optionally substituted heterocyclic fused ring, an optionally substituted alkoxy group, an optionally substituted amino group, an optionally substituted alkynyl group, an optionally substituted alkenyl group, an optionally substituted alkylcarbonyl group, a carboxy group, an alkoxycarbonyl group, an azido group, a nitrile group, an optionally substituted carbamoyl group, an optionally substituted thioether group, an optionally substituted alkylsulfonyl group, an optionally substituted sulfonamide group, a nitro group, or a formyl group; and

Q indicates a structure selected from the following structures (a) to (o):

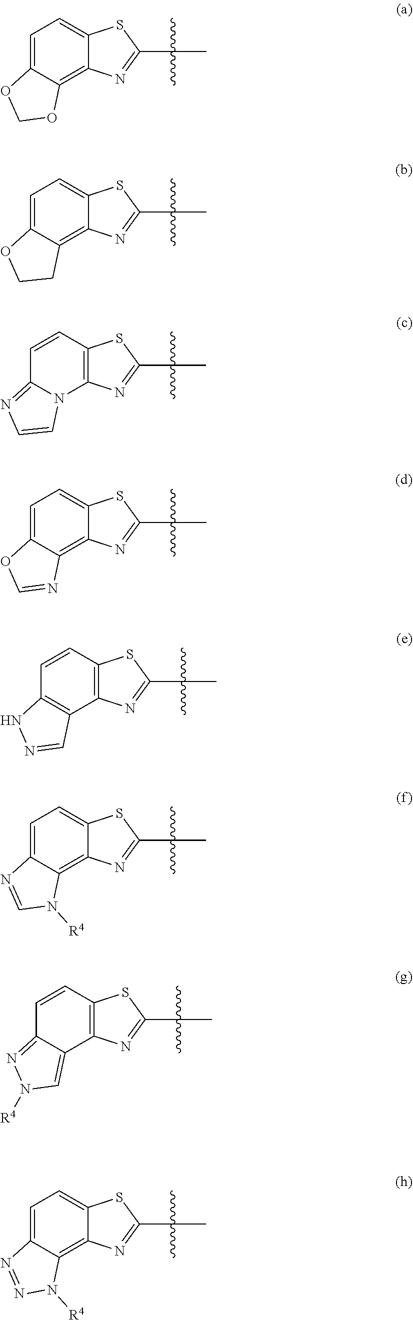

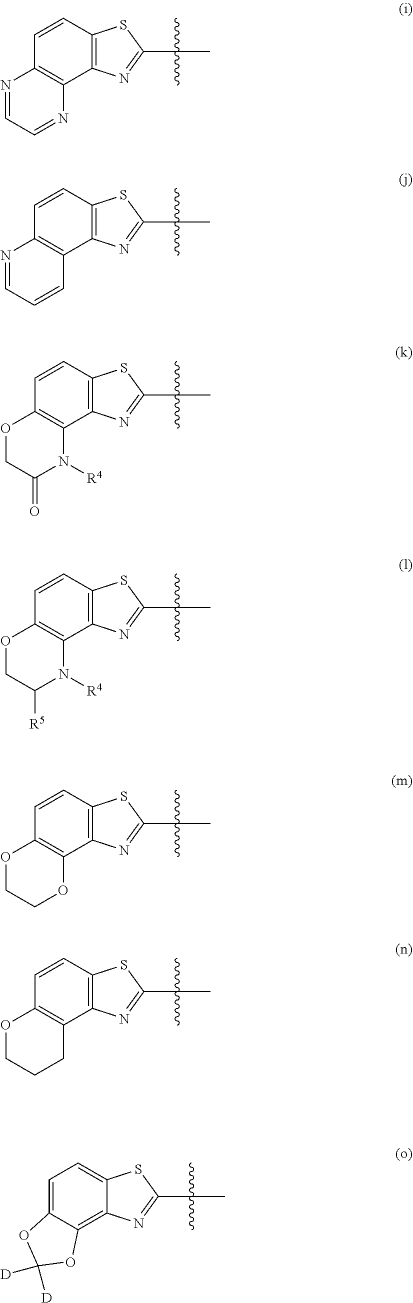

wherein R⁴ represents a hydrogen atom, an optionally substituted lower alkyl group, an optionally substituted cycloalkyl group, an optionally substituted alkylcarbonyl group, an optionally substituted alkylsulfonyl group, or an optionally substituted saturated heterocyclic group; and

R⁵ represents a hydrogen atom or an optionally substituted lower alkyl group,

or a pharmaceutically acceptable salt thereof.
(2) The alkyne derivative according to (1) above or a pharmaceutically acceptable salt thereof, wherein Q is selected from the structures (a) to (d) and (m) in formula (I) above.
(3) The alkyne derivative according to (2) above or a pharmaceutically acceptable salt thereof, wherein Q is structure (a) in formula (I) above.
(4) The alkyne derivative according to (2) above or a pharmaceutically acceptable salt thereof, wherein Q is structure (b) in formula (I) above.
(5) The alkyne derivative according to (2) above or a pharmaceutically acceptable salt thereof, wherein Q is structure (c) in formula (I) above.
(6) The alkyne derivative according to (2) above or a pharmaceutically acceptable salt thereof, wherein Q is structure (d) in formula (I) above.
(7) The alkyne derivative according to (2) above or a pharmaceutically acceptable salt thereof, wherein Q is structure (m) in formula (I) above.
(8) An alkyne derivative according to Examples 1 to 84 below or a pharmaceutically acceptable salt thereof.
(9) The alkyne derivative according to (1) above or a pharmaceutically acceptable salt thereof, which is selected from the group consisting of the following compounds:
(R)-1-([1,3]dioxolo[4′,5′:5,6]benzo[1,2-d]thiazol-7-yl)-5-(1-propyn-1-yl)imidazolidin-2-one (Example 2);
(4S,5R)-1-(7,8-dihydrobenzofuro[4,5-d]thiazol-2-yl)-4-methyl-5-(prop-1-yn-1-yl)imidazolidin-2-one (Example 5);
1-(7,8-dihydrobenzofuro[4,5-d]thiazol-2-yl)-5-(prop-1-yn-1-yl)imidazolidin-2-one (Example 9);
(4S,5R)-1-([1,3]dioxolo[4′,5′:5,6]benzo[1,2-d]thiazol-7-yl)-4-methyl-5-(prop-1-yn-1-yl)imidazolidin-2-one (Example 23);
(4S,5R)-1-(7,8-dihydro-[1,4]dioxyno[2′,3′:5,6]benzo[1,2-d]thiazol-2-yl)-4-methyl-5-(prop-1-yn-1-yl)imidazolidin-2-one (Example 56);
(4S,5R)-1-(8,9-dihydro-7H-chromeno[5,6-d]thiazol-2-yl)-4-methyl-5-(prop-1-yn-1-yl)imidazolidin-2-one (Example 57);
1-(7,8-dihydro-[1,4]dioxyno[2′,3′:5,6]benzo[1,2-d]thiazol-2-yl)-5-(prop-1-yn-1-yl)imidazolidin-2-one (Example 58);
(R)-1-([1,3]dioxolo[4′,5′:5,6]benzo[1,2-d]thiazol-7-yl-2,2-d2)-5-(prop-1-yn-1-yl)imidazolidin-2-one (Example 62);
cis-1-(7,8-dihydro-[1,4]dioxyno[2′,3′:5,6]benzo[1,2-d]thiazol-2-yl)-4-(hydroxymethyl)-5-(prop-1-yn-1-yl)imidazolidin-2-one (Example 64);
cis-1-(7,8-dihydrobenzofuro[4,5-d]thiazol-2-yl)-4-ethyl-5-(prop-1-yn-1-yl)imidazolidin-2-one (Example 73);
cis-1-(7,8-dihydrobenzofuro[4,5-d]thiazol-2-yl)-4-(methoxymethyl)-5-(prop-1-yn-1-yl)imidazolidin-2-one (Example 74);
(4R,5R)-1-(7,8-dihydrobenzofuro[4,5-d]thiazol-2-yl)-4-((R)-1-hydroxyethyl)-5-(prop-1-yn-1-yl)imidazolidin-2-one (Example 82); and
(4R,5R)-1-(7,8-dihydrobenzofuro[4,5-d]thiazol-2-yl)-4-((S)-1-hydroxyethyl)-5-(prop-1-yn-1-yl)imidazolidin-2-one (Example 84).
   (10) A medicament comprising the alkyne derivative according to any one of (1) to (9) above or a pharmaceutically acceptable salt thereof as an active ingredient.
   (11) A pharmaceutical composition comprising the alkyne derivative according to any one of (1) to (9) above or a pharmaceutically acceptable salt thereof as an active ingredient.
   (12) A therapeutic agent and/or a prophylactic agent for a disease involving DYRK, comprising the alkyne derivative according to any one of (1) to (9) above or a pharmaceutically acceptable salt thereof as an active ingredient.
   (13) The therapeutic agent and/or the prophylactic agent according to (12) above, wherein the disease involving DYRK is frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and mental retardation associated therewith, memory impairment, memory loss, learning disability, intellectual disability, cognitive impairment, mild cognitive impairment, treatment for progression of dementia symptoms or prevention of dementia onset, or brain tumor, pancreatic cancer, ovarian cancer, osteosarcoma, colorectal cancer, lung cancer, bone resorption disease, osteoporosis, sickle cell anemia, or bone resorption disease in chronic kidney disease.
   (14) A method for treating and/or preventing a disease involving DYRK, comprising administering to a patient in need of the treatment a therapeutically effective amount of the alkyne derivative according to any one of (1) to (9) above or a pharmaceutically acceptable salt thereof.
   (15) Use of the alkyne derivative according to any one of (1) to (9) above or a pharmaceutically acceptable salt thereof, for producing a therapeutic agent and/or a prophylactic agent for a disease involving DYRK.
   (16) The alkyne derivative according to any one of (1) to (9) above or a pharmaceutically acceptable salt thereof, for use in the treatment and/or prevention of a disease involving DYRK.
   (17) A medicament comprising the medicament according to (10) above in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.
   (18) The medicament according to (17) above, for treating frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and a complication associated therewith, mental retardation, memory impairment, memory loss, learning disability, intellectual disability, cognitive impairment, mild cognitive impairment, or treatment for progression of dementia symptoms or prevention of dementia onset; or treating brain tumor, pancreatic cancer, ovarian cancer, osteosarcoma, colorectal cancer, lung cancer, bone resorption disease, osteoporosis, sickle cell anemia, or bone resorption disease in chronic kidney disease in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.

### Effect of the Invention

The present inventors have intensively studied so as to achieve the above object and found that a novel alkyne derivative of formula (I) and a pharmaceutically acceptable salt thereof have an excellent DYRK inhibitory effect, and thus completed the present invention. Compounds provided by the present invention are useful as a therapeutic agent for diseases which are known to be involved in abnormal cell response through DYRK1A, for example, Alzheimer's disease, Parkinson's disease, Down's syndrome, neuropsychiatric disorder such as depression, mental retardation associated therewith, memory impairment, memory loss, learning disability, intellectual disability, cognitive impairment, mild cognitive impairment, progression of dementia symptoms or a prophylactic agent for dementia onset; and a prophylactic or therapeutic pharmaceutical (pharmaceutical composition) for tumors such as brain tumors. The compounds provided by the present invention are DYRK1B inhibitors that are useful as a prophylactic or therapeutic pharmaceutical (pharmaceutical composition) for tumors such as pancreatic cancer, ovarian cancer, osteosarcoma, colorectal cancer, and lung cancer. Since DYRK2 controls p53 to induce apoptosis in response to DNA damages, the compounds provided by the present invention are further useful as a prophylactic or therapeutic pharmaceutical (pharmaceutical composition) for bone resorption disease and osteoporosis. The compounds provided by the present invention are also DYRK3 inhibitors that are useful as a prophylactic or therapeutic pharmaceutical (pharmaceutical composition) for sickle cell anemia, bone resorption disease in chronic kidney disease, and osteoporosis. The compounds are also useful, as a compound inhibiting DYRK, for reagents to be used in pathological imaging and for reagents for basic experiments and research regarding the above diseases.

### BEST MODE TO CARRY OUT THE INVENTION

The present invention will be described in detail below.

The novel alkyne derivative of the present invention is a compound of the following formula (I):

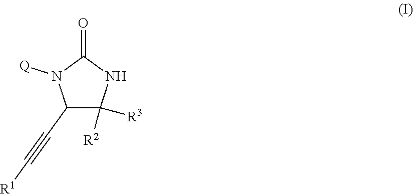

wherein R¹ represents a hydrogen atom, a halogen atom, a trimethylsilyl group, an optionally substituted aryl group, an optionally substituted heteroaryl group, an optionally substituted lower alkyl group, and an optionally substituted cycloalkyl group;

R² and R³ represent each independently a hydrogen atom, a halogen atom, an optionally substituted lower alkyl group, an optionally substituted cycloalkyl group, an optionally substituted aryl group, an optionally substituted heteroaryl group, an optionally substituted saturated heterocyclic group, an optionally substituted heterocyclic fused ring, an optionally substituted alkoxy group, an optionally substituted amino group, an optionally substituted alkynyl group, an optionally substituted alkenyl group, an optionally substituted alkylcarbonyl group, a carboxy group, an alkoxycarbonyl group, an azido group, a nitrile group, an optionally substituted carbamoyl group, an optionally substituted thioether group, an optionally substituted alkylsulfonyl group, an optionally substituted sulfonamide group, a nitro group, or a formyl group; and

Q is a structure selected from the following structures (a) to (o):

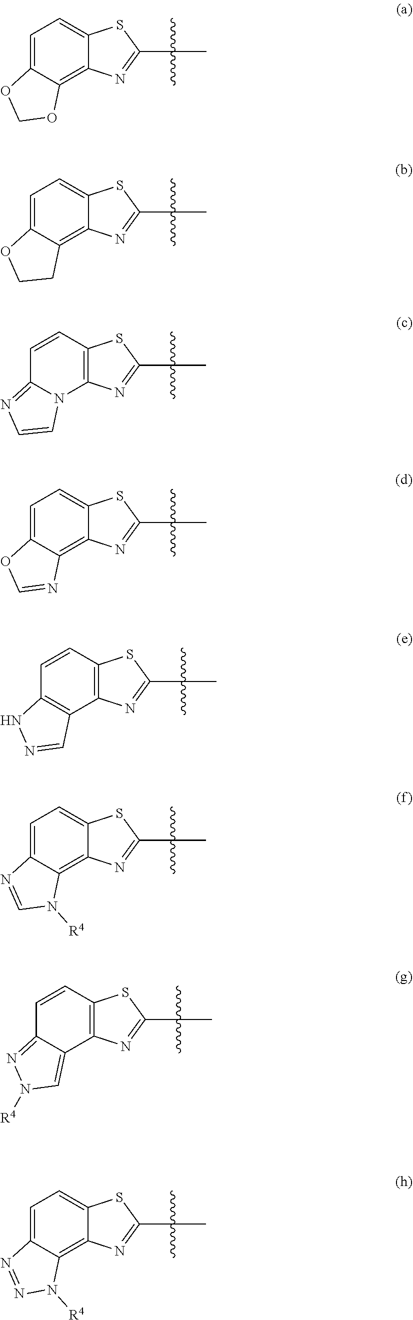

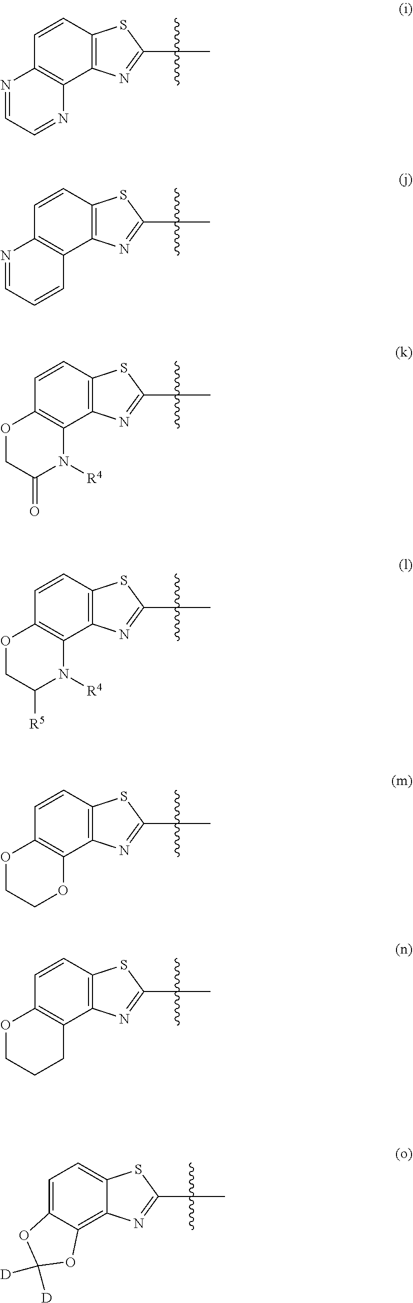

wherein R⁴ represents a hydrogen atom, an optionally substituted lower alkyl group, an optionally substituted cycloalkyl group, an optionally substituted alkylcarbonyl group, an optionally substituted alkylsulfonyl group, and an optionally substituted saturated heterocyclic group; and

R⁵ represents a hydrogen atom or an optionally substituted lower alkyl group.

The term “DYRK” represents a dual-specificity tyrosine-phosphorylation regulated protein kinase and means one or more DYRK family members (DYRK1A, DYRK1B, DYRK2, DYRK3, and DYRK4).

The term “lower alkyl group” means a linear or branched saturated hydrocarbon group of 1 to 6 carbon atoms (C₁₋₆ alkyl group). The lower alkyl group preferably includes a “C₁₋₄ alkyl group” and more preferably a “C₁₋₃ alkyl group”. Specific examples of the “lower alkyl group” include a methyl group, an ethyl group, an n-propyl group, a 1-methylethyl group, an n-butyl group, a tert-butyl group, a 1-methylpropyl group, a 2-methylpropyl group, an n-pentyl group, a 1,1-dimethylpropyl group, a 1,2-dimethylpropyl group, a 1-methylbutyl group, a 2-methylbutyl group, a 4-methylpentyl group, a 3-methylpentyl group, a 2-methylpentyl group, a 1-methylpentyl group, and a hexyl group.

The term “cycloalkyl group” means a cyclic saturated hydrocarbon group of 3 to 10 carbon atoms and also includes those having a partially unsaturated bond and a cross-linked structure. The “cycloalkyl group” preferably includes a “C₃₋₇ cycloalkyl group” and more preferably a “C₃₋₆ cycloalkyl group”. Specific examples of the “cycloalkyl group” include a cyclopropyl group, a cyclobutyl group, a cyclopentyl group, a cyclohexyl group, a cycloheptyl group, a cyclooctyl group, a cyclononyl group, a cyclodecyl group, and an adamantyl group.

The term “aryl group” means an aromatic cyclic group of 6 to 14 carbon atoms. The “aryl group” preferably includes a “C₆₋₁₀ aryl group” and more preferably a “C₆ aryl group”. Specific examples of the “aryl group” include a phenyl group and a naphthyl group.

The term “heteroaryl group” means a 5- to 10-membered heterocyclic aromatic cyclic group containing at least one heteroatom selected from the group consisting of a nitrogen atom, a sulfur atom, and an oxygen atom. The “heteroaryl group” preferably includes a 5- to 8-membered heteroaryl group and more preferably a 5- or 6-membered heteroaryl group. Specific examples of the “heteroaryl group” include an imidazolyl group, a pyrazolyl group, a thiazolyl group, a thienyl group, a furyl group, a pyrrole group, and a pyridyl group.

The term “saturated heterocyclic group” means a 3- to 8-membered saturated or partially unsaturated monocyclic heterocyclic group containing at least one heteroatom selected from the group consisting of a nitrogen atom, a sulfur atom, and an oxygen atom. The “saturated heterocyclic group” preferably includes a 3- to 6-membered saturated heterocyclic group and more preferably a 5- or 6-membered heterocyclo group. Specific examples of the “saturated heterocyclic group” include an epoxy group, an oxetanyl group, a tetrahydrofuranyl group, a tetrahydropyranyl group, an azetidinyl group, a pyrrolidinyl group, a piperidinyl group, a piperazinyl group, a morpholinyl group, and a thiomorpholinyl group.

The term “heterocyclic fused-ring group” means a bicyclic cyclic group with a fused 3- to 8-membered ring, which is a fused heterocyclic group having a 3- to 8-membered alicyclic or aromatic cyclic heterocyclic group comprising at least one heteroatom selected from the group consisting of a nitrogen atom, a sulfur atom, and an oxygen atom. The “heterocyclic fused-ring group” preferably includes a heterocyclic fused-ring group having a 3- to 6-membered alicyclic or aromatic cyclic heterocyclic group, and more preferably a heterocyclic fused-ring group having a 5- or 6-membered alicyclic or aromatic cyclic heterocyclic group. Specific examples of the “heterocyclic fused-ring group” include a tetrahydroisoquinolyl group, a benzothiophenyl group, a benzimidazolyl group, a benzoxazolyl group, a benzothiazolyl group, an indolyl group, an isoquinolyl group, and a phthalimide group.

The term “alkoxy group” means an oxy group substituted with the “lower alkyl group” or a 3- to 6-membered cyclic alkyl group. The “alkoxy group” preferably includes a “C₁₋₆ alkoxy group” and more preferably a “C₁₋₃ alkoxy group”. Specific examples of the “alkoxy group” include a methoxy group, an ethoxy group, a propoxy group, a 1-methylethoxy group, a butoxy group, a 1,1-dimethylethoxy group, a 1-methylpropoxy group, a 2-methylpropoxy group, a pentyloxy group, a 1,1-dimethylpropoxy group, a 1,2-dimethylpropoxy group, a 1-methylbutoxy group, a 2-methylbutoxy group, a 4-methylpentyloxy group, a 3-methylpentyloxy group, a 2-methylpentyloxy group, a 1-methylpentyloxy group, a hexyloxy group, and a cyclopropyloxy group.

The term “alkynyl group” means a linear or branched saturated hydrocarbon group of 2 to 6 carbon atoms having 1 to 3 triple bonds (C₂₋₆ alkynyl group). The “alkynyl group” preferably includes a “C₂₋₅ alkynyl group” and more preferably a “C₂₋₄ alkynyl group”. Specific examples of the “alkynyl group” include an ethynyl group, a propargyl group, and a 2-butynyl group.

The term “alkenyl group” means a linear or branched saturated hydrocarbon group of 2 to 6 carbon atoms having 1 to 3 double bonds (C₂₋₆ alkenyl group). The “alkenyl group” preferably includes a “C₂₋₅ alkenyl group” and more preferably a “C₂₋₄ alkenyl group”. Specific examples of the “alkenyl group” include a vinyl group, an allyl group, a 1-propenyl group, an isopropenyl group, and a 2-methylallyl group.

The term “alkylcarbonyl group” means a carbonyl group substituted with the “lower alkyl group” or a 3- to 6-membered cyclic alkyl group, and examples thereof include an acetyl group.

The term “alkylsulfonyl group” means a sulfonyl group substituted with the “lower alkyl group” or a 3- to 6-membered cyclic alkyl group, and examples thereof include a methylsulfonyl group.

Examples of the optionally substituted sulfonamide group include a methylsulfonamide group and an ethylsulfonamide group.

The optionally substituted amino group may be, for example, any amino groups having a linear, branched, or cyclic alkyl group of 1 to 3 carbon atoms, and specific examples thereof include an amino group, a methylamino group, and a dimethylamino group.

Examples of the optionally substituted carbamoyl group include a methylcarbamoyl group, an ethylcarbamoyl group, and a dimethylcarbamoyl group.

The optionally substituted thioether group may be, for example, any thioether groups having a linear, branched, or cyclic alkyl group of 1 to 3 carbon atoms, and specific examples thereof includes a methylsulfanyl group, an ethylsulfanyl group, an isopropylsulfanyl group, and a cyclopropylsulfanyl group.

The alkoxycarbonyl group include, for example, a methoxycarbonyl group and an ethoxycarbonyl group.

For a “substituent” of the optionally substituted lower alkyl group, the optionally substituted cycloalkyl group, the optionally substituted saturated heterocyclic group, the optionally substituted alkoxy group, the optionally substituted amino group, the optionally substituted alkynyl group, the optionally substituted alkenyl group, the optionally substituted alkylcarbonyl group, the optionally substituted carbamoyl group, the optionally substituted thioether group, the optionally substituted alkylsulfonyl group, or the optionally substituted sulfonamide group, the group may have one or more of any type of substituents at any chemically allowable position, and when the above group has two or more substituents, the substituents may be the same or different, unless otherwise specified. The substituent specifically includes, for example, a C₃₋₆ cycloalkyl group, a halogen atom, a C₁₋₄ alkoxy group, a cyano group, a benzyloxy group, a phenyl group, a hydroxy group, a methanesulfonyl group, and a substituted or unsubstituted amino group.

For a “substituent” of the optionally substituted aryl group, the optionally substituted heteroaryl group, and the optionally substituted heterocyclic fused ring, the group or ring may have one or more of any type of substituents at any chemically allowable position, and when the above group has two or more substituents, the substituents may be the same or different, unless otherwise specified. The substituent specifically includes, for example, a halogen atom, a vinyl group, a methoxy group, a cyano group, a hydroxy group, and a hydroxymethyl group.

As used herein, a halogen atom refers to a chlorine atom (Cl), a bromine atom (Br), a fluoride atom (F), and iodine atom (I), and in particular, is preferably Cl, Br, and F.

Compounds of the present invention represented by formula (I) include R¹, R², R³, R⁴, R⁵, and Q whose definition and preferred scope are each described as follows, but the technical scope of the present invention is not limited to the scope of the compounds mentioned below:

R¹ is preferably a hydrogen atom, an optionally substituted lower alkyl group, or an optionally substituted cycloalkyl group, and more preferably an optionally substituted lower alkyl group;
R² is preferably a hydrogen atom or an optionally substituted lower alkyl group, and more preferably a hydrogen atom;
R³ is preferably an optionally substituted lower alkyl group or a hydrogen atom, and more preferably an optionally substituted lower alkyl group;
R⁴ is preferably an optionally substituted lower alkyl group;
R⁵ is preferably a hydrogen atom; and
Q is preferably (a), (b), (c), (d), (f), (g), (i), (j), (m), (n), or (o) and more preferably (a), (b), (d), (g), (m), (n), or (o).

Among the compounds of the present invention represented by formula (I), preferred compounds include the following alkyne derivative or a pharmaceutically acceptable salt thereof:

a compound in which R¹ is a hydrogen atom or a “C₁₋₄ alkyl group”; R² is a hydrogen atom or a “C₁₋₄ alkyl group”; R³ is a hydrogen atom or a “C₁₋₄ alkyl group”; and

### Q is (a), (b), (d), (m), (n), or (o).

More preferred compounds include the following alkyne derivative or a pharmaceutically acceptable salt thereof:

a compound in which R¹ is a “C₁₋₃ alkyl group”; R² is a hydrogen atom or “C₁₋₃ alkyl group”; R³ is a hydrogen atom; and

### Q is (a), (b), (m), or (o).

Further preferred compounds include the following compound or a pharmaceutically acceptable salt thereof:

a compound in which R¹ is a “C₁₋₃ alkyl group”; R² is a “C₁₋₃ alkyl group”; R³ is a hydrogen atom; and

### Q is (a), (b), or (m).

Among the compounds of the present invention represented by formula (I), preferred compounds specifically include the following alkyne derivative or a pharmaceutically acceptable salt thereof:
(R)-1-([1,3]dioxolo[4′,5′:5,6]benzo[1,2-d]thiazol-7-yl)-5-(1-propyn-1-yl)imidazolidin-2-one (Example 2);
(4S,5R)-1-(7,8-dihydrobenzofuro[4,5-d]thiazol-2-yl)-4-methyl-5-(prop-1-yn-1-yl)imidazolidin-2-one (Example 5);
1-(7,8-dihydrobenzofuro[4,5-d]thiazol-2-yl)-5-(prop-1-yn-1-yl)imidazolidin-2-one (Example 9);
(4S,5R)-1-([1,3]dioxolo[4′,5′:5,6]benzo[1,2-d]thiazol-7-yl)-4-methyl-5-(prop-1-yn-1-yl)imidazolidin-2-one (Example 23);
(4S,5R)-1-(7,8-dihydro-[1,4]dioxyno[2′,3′:5,6]benzo[1,2-d]thiazol-2-yl)-4-methyl-5-(prop-1-yn-1-yl)imidazolidin-2-one (Example 56);
(4S,5R)-1-(8,9-dihydro-7H-chromeno[5,6-d]thiazol-2-yl)-4-methyl-5-(prop-1-yn-1-yl)imidazolidin-2-one (Example 57);
1-(7,8-dihydro-[1,4]dioxyno[2′,3′:5,6]benzo[1,2-d]thiazol yl)-5-(prop-1-yn-1-yl)imidazolidin-2-one (Example 58);
(R)-1-([1,3]dioxolo[4′,5′:5,6]benzo[1,2-d]thiazol-7-yl-2,2-d2)-5-(prop-1-yn-1-yl)imidazolidin-2-one (Example 62);
cis-1-(7,8-dihydro-[1,4]dioxyno[2′,3′:5,6]benzo[1,2-d]thiazol-2-yl)-4-(hydroxymethyl)-5-(prop-1-yn-1-yl)imidazolidin-2-one (Example 64);
cis-1-(7,8-dihydrobenzofuro[4,5-d]thiazol-2-yl)-4-ethyl-5-(prop-1-yn-1-yl)imidazolidin-2-one (Example 73);
cis-1-(7,8-dihydrobenzofuro[4,5-d]thiazol-2-yl)-4-(methoxymethyl)-5-(prop-1-yn-1-yl)imidazolidin-2-one (Example 74);
(4R,5R)-1-(7,8-dihydrobenzofuro[4,5-d]thiazol-2-yl)-4-((R)-1-hydroxyethyl)-5-(prop-1-yn-1-yl)imidazolidin-2-one (Example 82); and
(4R,5R)-1-(7,8-dihydrobenzofuro[4,5-d]thiazol-2-yl)-4-((S)-1-hydroxyethyl)-5-(prop-1-yn-1-yl)imidazolidin-2-one (Example 84).

Examples of the pharmaceutically acceptable salt of compound (I) of the present invention include inorganic acid salts with hydrochloric acid, sulfuric acid, carbonic acid, and phosphoric acid; and organic acid salts with fumaric acid, maleic acid, methanesulfonic acid, and p-toluenesulfonic acid. The present invention also encompasses ammonium salts, in addition to salts with an alkali metal such as sodium and potassium; with an alkaline earth metal such as magnesium and calcium; with an organic amine such as lower alkylamine and lower alcoholamine; and with a basic amino acid such as lysine, arginine, and ornithine.

Isomers may exist in compound (I) of the present invention, for example, depending on the type of the substituent. The isomers may be herein described by a chemical structure of only one form thereof, but the present invention encompasses all isomers (geometrical isomer, stereoisomer, tautomer, etc.) which can be structurally formed, and also encompasses isomers alone, or a mixture thereof. In the present invention, the “hydrogen atom” includes ¹H and ²H (D), and the compound represented by formula (I) also encompasses a deuterium converter in which either one or more ¹H of the compound represented by formula (I) has been converted into ²H (D).

Compound (I) or a pharmaceutically acceptable salt thereof of the present invention can be produced, for example, by the methods described below. When a defined group is chemically affected under the conditions of an exemplified method in the production method shown below or is unsuitable for use to carry out the method, it is possible to easily produce them by a method which is usually used in organic synthetic chemistry, for example, a method of applying means such as protection or deprotection of a functional group [T. W. Greene, Protective Groups in Organic Synthesis 3rd Edition, John Wiley & Sons, Inc., 1999]. If necessary, the order of reaction steps such as introduction of substituents cab also be changed.

Meanings of abbreviations and symbols used in the following description are as follows.

DCM: dichloromethane
THF: tetrahydrofuran
DMF: N,N-dimethyl formamide
TEA: triethylamine
EtOH: ethanol
LAH: lithium aluminum hydride
DMA: N,N-dimethyl acetamide
LDA: lithium diisopropyl amide

### [Preparative Method of Compound (I) of the Present Invention]

The compound of the present invention represented by the formula (I) can be produced, for example, according to Scheme 1:

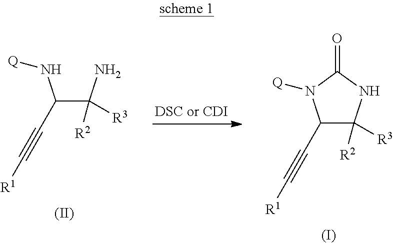

wherein R¹, R², R³, and Q are the same as defined in the above (I).

Compound (I) of the present invention can be obtained by reacting compound (II) with a condensing reagent such as 1,1′-carbonyldiimidazole (CDI) and di(N-succinimidyl) carbonate (DSC) in a solvent. Although the condensing reagent can be used in an excess amount, compound (I) can be synthesized by reacting compound (II) with preferably 1 to 5 molar equivalents of CDI or DSC. Any solvent may be used as long as it is inert to the reaction. Although it is not particularly limited, for example, THF, DMF, and DMA can be used, and DMF can be preferably used. The reaction can be carried out within several minutes to several hours at a temperature ranging from 0° C. to room temperature, and it is possible to synthesize the product preferably by reacting approximately for 30 minutes to 1 hour at room temperature.

Compound (II) to be used as a raw material of Scheme 1 can be produced, for example, by the method shown in Scheme 2:

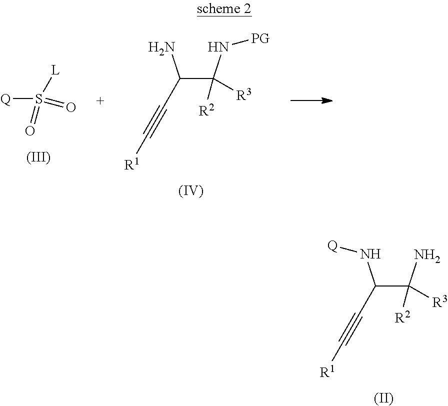

wherein R¹, R², R³, and Q are the same as defined in the above (I); L represents a lower alkyl group; and PG represents a protecting group.

Compound (II) can be produced by reacting compound (III) with amine (IV) in a solvent such as THF, acetonitrile, or DMA, or in the absence of the solvent, followed by deprotection. In other words, compound (II) can be synthesized by reacting compound (III) with 3 to 10 molar equivalents of amine (IV) so as to protect an amino group of compound (II). The reaction can be carried out within several minutes to several days at a temperature ranging from room temperature to 150° C., and it is possible to synthesize the product preferably by reacting for several hours to 24 hours at 80° C. to 120° C. Compound (II) can be obtained by deprotecting the protecting group in the amino group under a condition generally used in organic chemistry.

A compound (II-b), in which Q has a structure (b), of compound (II) to be used as a raw material of Scheme 1 can be produced, for example, by the method shown in Scheme 3:

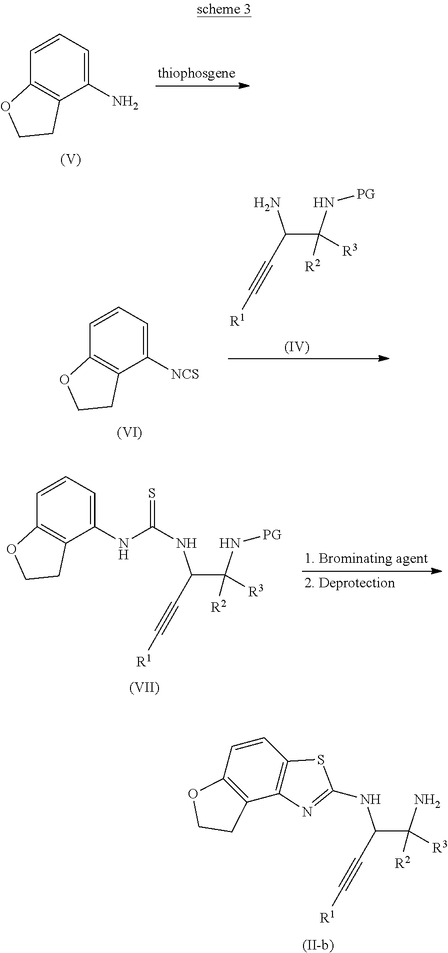

wherein R¹, R², and R³ are the same as defined in the above (I); and PG represents a protecting group.

Compound (II-b) can be produced by converting aniline (V) into isothiocyanate (VI), then treating thiourea (VII) synthesized by reacting isothiocyanate (VI) with amine (IV) using a brominating agent, followed by deprotection. In other words, the isothiocyanate (VI) can be obtained by reacting the aniline (V) with 1 to 10 molar equivalents of thiophosgene in an aqueous solution. The reaction can be carried out within several minutes to 24 hours at a temperature ranging from −30° C. to room temperature, and it is possible to synthesize the product preferably by reacting for 1 hour to 4 hours at −10° C. to 0° C.

Thiourea (VII) can be obtained by reacting the resulting isothiocyanate (VI) with 1 to 1.5 molar equivalents of amine (IV) in the presence or absence of a base such as sodium ethoxide in a solvent such as ethanol. The reaction can be carried out within several minutes to several days at a temperature ranging from 0° C. to room temperature, and it is possible to synthesize the product preferably by reacting for several hours to 24 hours at 10° C. to room temperature.

Compound (II-b) having a protected amino group can be obtained by reacting the resulting thiourea (VII) with a large excess of acetic acid and 0.9 to 1 molar equivalents of bromine in a solvent such as acetonitrile or DCM. Compound (II-b) having the protected amino group can also be synthesized by reacting thiourea (VII) with 5 to 10 molar equivalents of sodium hydrogencarbonate and a brominating agent such as 0.9 to 1 molar equivalents of benzyltrimethylammonium tribromide. The reaction can be carried out within several minutes to several days at a temperature ranging from 0° C. to room temperature, and it is possible to synthesize the product preferably by reacting for several hours to 24 hours at 10° C. to room temperature. Compound (II-b) can also be obtained by deprotecting the protecting group in the amino group under a condition generally used in organic chemistry.

The compound of formula (I), in which Q has structures (a) and (c) to (o), can be produced in the same manner by using other corresponding aniline derivatives in place of aniline (V) of Scheme 3.

A compound (II-c), in which Q has a structure (c), of compound (II) to be used as a raw material of Scheme 1 can be produced for example, by the method shown in Scheme 4:

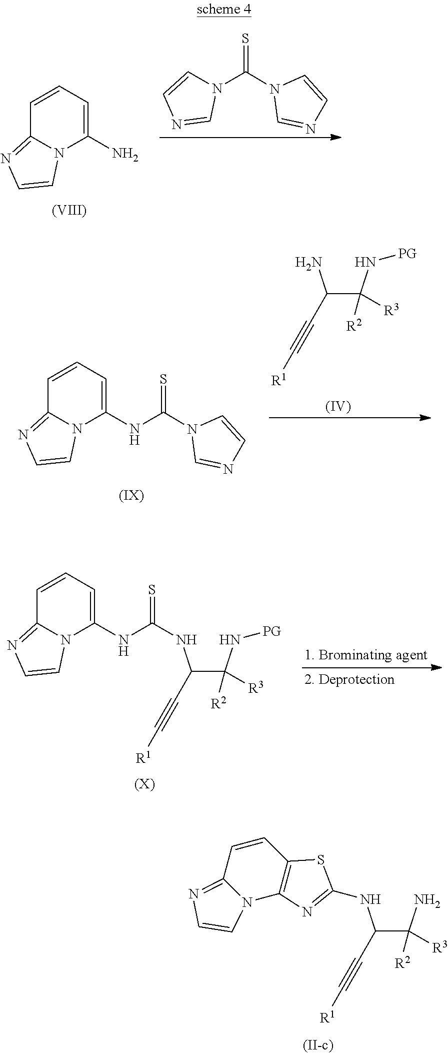

wherein R¹, R², and R³ are the same as defined in the above (I); and PG represents a protecting group.

Compound (II-c) can be produced by reacting thiourea (X), obtained by reacting thiocarbonylimidazole (IX) prepared from amine (VIII) and 1,1′-dithiocarbonyldiimidazole with amine (IV), with a brominating agent to form a thiazole ring in a solvent, followed by deprotection. In other words, the thiocarbonylimidazole (IX) can be obtained by reacting the amine (VIII) with 1 to 10 molar equivalents of 1,1′-dithiocarbonyldiimidazole in a solvent such as THF or DCM. The reaction can be carried out within several minutes to 24 hours at a temperature ranging from −30° C. to 60° C., and it is possible to synthesize the product preferably by reacting for 1 hour to 24 hours at 10° C. to room temperature.

Thiourea (X) can be obtained by reacting the resulting thiocarbonylimidazole (IX) with 1 to 1.5 molar equivalents of amine (IV) in a solvent such as THF. The reaction can be carried out within several minutes to 24 hours at a temperature ranging from 0° C. to room temperature, and it is possible to synthesize the product preferably by reacting for 30 minutes to 2 hours at 10° C. to room temperature.

Compound (II-c) having a protected amino group can be obtained by reacting the resulting thiourea (X) with large excess acetic acid and 0.9 to 1 molar equivalents of bromine in a solvents such as acetonitrile. The reaction can be carried out within several minutes to 18 hours at a temperature ranging from 0° C. to room temperature, and it is possible to synthesize the product preferably by reacting for 30 minutes to 2 hours at 10° C. to room temperature. Compound (II-c) having the protected amino group can also be synthesized by reacting thiourea (X) with 5 to 10 molar equivalents of sodium hydrogencarbonate and a brominating reagent such as 0.9 to 1 molar equivalents of benzyltrimethylammonium tribromide. The reaction can be carried out within several minutes to several days at a temperature ranging from 0° C. to room temperature, and it is possible to synthesize the product preferably by reacting for several hours to 24 hours at 10° C. to room temperature. Compound (II-c) can be obtained by deprotecting a protecting group in the amino group under a condition generally used in organic chemistry.

The compound of formula (I), in which Q has structures (a), (b), and (d) to (o), can also be produced in the same manner by using other corresponding aniline derivatives in place of to amine (VIII) of Scheme 4.

Compound (II-d), in which Q has structure (d), of compound (II) to be used as a raw material of Scheme 1 can be produced, for example, by the method shown in Scheme 5:

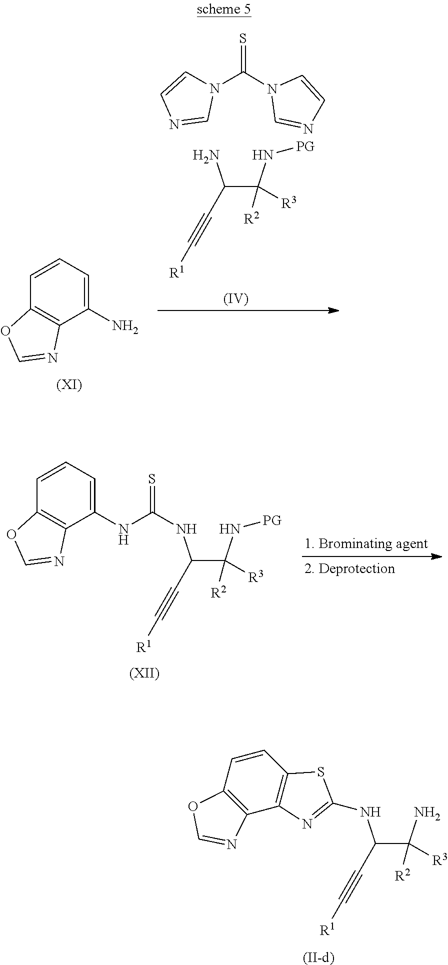

wherein R¹, R², and R³ are the same as defined in the above (I); and PG represents a protecting group.

Compound (II-d) can be produced by reacting thiourea (XII), obtained by reacting amine (XI) with 1,1′-dithiocarbonyldiimidazole and amine (IV), with a brominating agent to form a thiazole ring in a solvent, followed by deprotection. In other words, thiourea (XII) can be obtained by adding 1 to 5 molar equivalents of 1,1′-dithiocarbonyldiimidazole and 1 to 5 molar equivalents of amine (XI) at the same time to amine (IV) in a solvent such as THF and then reacting them. The reaction can be carried out within several minutes to 24 hours at a temperature ranging from −30° C. to 60° C., and it is possible to synthesize the product preferably by reacting for 30 minutes to 4 hours at 10° C. to room temperature.

Compound (II-d) having a protected amino group can be obtained by reacting the resulting thiourea (XII) with a large excess of acetic acid and 0.9 to 1 molar equivalents of bromine in solvents such as acetonitrile and DCM. Compound (II-d) having the protected amino group can also be obtained by reacting thiourea (XII) with 5 to 10 molar equivalents of sodium hydrogencarbonate and a brominating agent such as 0.9 to 1 molar equivalents of benzyltrimethylammonium tribromide. The reaction can be carried out within several minutes to 18 hours at a temperature ranging from −30° C. to room temperature, and it is possible to synthesize the product preferably by reacting for 30 minutes to 2 hours at −10° C. to 0° C. Compound (II-d) can be obtained by deprotecting a protecting group in the amino group under a condition generally used in organic chemistry.

The compound of formula (I), in which Q has structures (a) to (c) and (e) to (o), can also be produced in the same manner by using other corresponding aniline derivatives in place of to amine (XI) of Scheme 5.

Amine (IV) to be used as a raw material of Schemes 2 to 5 can be produced, for example, by the method shown in Scheme 6:

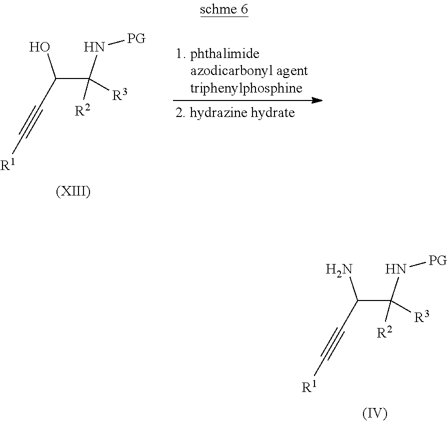

wherein R¹, R², and R³ are the same as defined in the above (I); and PG represents a protecting group.

The amine (IV) can be produced by converting a hydroxy group of compound (XIII) into a phthaloyl group using the Mitsunobu reaction and deprotecting phthalimide. In other words, phthaloyl-protected amine (IV) can be obtained by reacting compound (XIII) with 1 to 5 molar equivalents of diethyl azodicarboxylate, 1 to 5 molar equivalents of triphenylphosphine, and 1 to 5 molar equivalents of phthalimide in a solvent such as THF. The reaction can be carried out within several minutes to 24 hours at a temperature ranging from −30° C. to room temperature, and it is possible to synthesize the product preferably by reacting for 2 hours to 24 hours at −10° C. to 0° C. Amine (IV) can be obtained by reacting the resulting phthaloyl-protected amine (IV) with a large excess of hydrazine hydrate in a solvent such as ethanol. The reaction can be carried out within several minutes to 24 hours at a temperature ranging from room temperature to 100° C., and it is possible to synthesize the product preferably by reacting for 2 hours to 24 hours at 40° C. to 70° C.

Compound (XIII) to be used as a raw material of Scheme 6 can be produced, for example, by the method shown in Scheme 7:

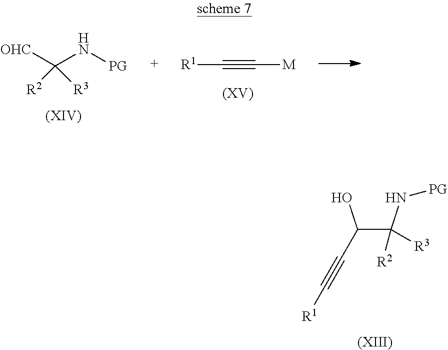

wherein R¹, R², and R³ are the same as defined in the above (I); M represents a metal such as lithium and magnesium; and PG represents a protecting group.

Compound (XIII) can be produced by the Grignard reaction of aldehyde (XIV) with alkyne (XV). In other words, compound (XIII) can be obtained by reacting aldehyde (XIV) with a Grignard reagent prepared from 5 to 10 molar equivalents of alkyne (XV) in a solvent such as THF. The reaction can be carried out within several minutes to 24 hours at a temperature ranging from −80° C. to room temperature, and it is possible to synthesize the product preferably by reacting for 30 minutes to 2 hours at −80° C. to −20° C.

Aldehyde (XIV) to be used as a raw material of Scheme 7 can be obtained as a commercially available product or produced by a known method or a method which is usually used in organic synthetic chemistry.

Compound (XIII) to be used as a raw material of Scheme 6 can be produced, for example, by the method shown in Scheme 8:

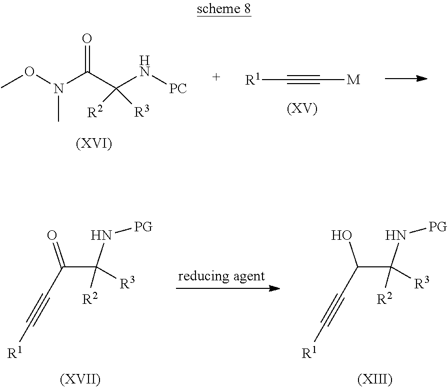

wherein R¹, R², and R³ are the same as defined in the above (I); M represents a metal such as lithium and magnesium; and PG represents a protecting group.

Compound (XIII) can be produced by reducing ketone (XVII) obtained by the reaction of Weinreb amide (XVI) with alkyne (XV) as a Grignard reagent. In other words, ketone (XVII) can be obtained by reacting Weinreb amide (XVI) with the Grignard reagent prepared from 5 to 10 molar equivalents of the alkyne (XV) in a solvent such as THF. The reaction can be carried out within several minutes to 24 hours at a temperature ranging from −80° C. to room temperature, and it is possible to synthesize the product preferably by reacting for 1 hour to 4 hours at −80° C. to −20° C. Compound (XIII) can be obtained by reacting the resulting ketone (XVII) with 1 to 5 molar equivalents of a reducing agent such as sodium borohydride or borane complex in the presence or absence of a catalyst in a solvent such as methanol or THF. The reaction can be carried out within several minutes to 24 hours at a temperature ranging from −80° C. to room temperature, and it is possible to synthesize the product preferably by reacting for 30 minutes to 2 hours at −20° C. to 0° C.

Weinreb amide (XVI) to be used as a raw material of Scheme 8 can be obtained as a commercially available product or produced by a known method or a method which is usually used in organic synthetic chemistry.

Compound (III-a), in which Q has a structure (a), of compound (III) to be used as a raw material of Scheme 2 can be produced, for example, by the method shown in Scheme 9:

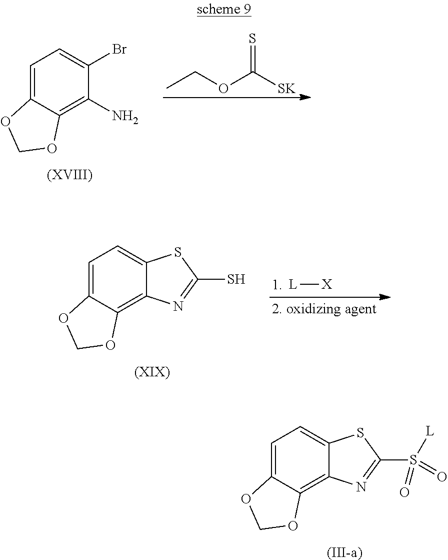

wherein L represents a lower alkyl group; and X represents halogen.

Compound (III-a) can be produced by alkylating mercaptobenzothiazole (XIX), obtained by cyclization of bromoaniline (XVIII) with potassium ethylxanthate, with halogenated alkyl and then oxidizing the resulting alkylthioether with an oxidizing agent. In other words, mercaptobenzothiazole (XIX) can be obtained by reacting bromoaniline (XVIII) with 2.5 to 3 molar equivalents of potassium ethylxanthate under heating in a solvent such as DMF, for example. The reaction can be carried out within several minutes to several days at a temperature ranging from 90° C. to reflux temperature, and it is possible to synthesize the product preferably by reacting for 1 day to 2 days at 100° C. to 120° C. The corresponding alkylthioether can be obtained by reacting the resulting mercaptobenzothiazole (XIX) with 3 to 4 molar equivalents of halogenated alkyl in the presence of base such as potassium carbonate in a solvent such as DMF, for example. The reaction can be carried out within several minutes to several days at a temperature ranging from 0° C. to room temperature, and it is possible to synthesize the product preferably by reacting for 30 minutes to 4 hours at 10° C. to room temperature. Compound (III-a) can be obtained by oxidizing the resulting alkylthioether, 0.8 to 2.5 molar equivalents of meta-chloroperoxybenzoic acid (m-CPBA), a peroxide such as hydrogen peroxide, KMnO₄ (potassium permanganate), or other oxidizing agents usually used in organic synthesis in a solvent such as acetic acid, water, or DCM, for example. The reaction can be carried out within 10 minutes to 2 days at a temperature ranging from 0° C. to room temperature, and it is possible to synthesize the product preferably by reacting for 10 minutes to 2 hours at 10° C. to room temperature.

The compound of formula (I), in which Q has structures (b) to (o), can be produced in the same manner by using other corresponding bromoaniline derivatives in place of bromoaniline (XVIII) of Scheme 9.

The compound of the present invention represented by formula (I) can also be produced, for example, by the method shown in Scheme 10:

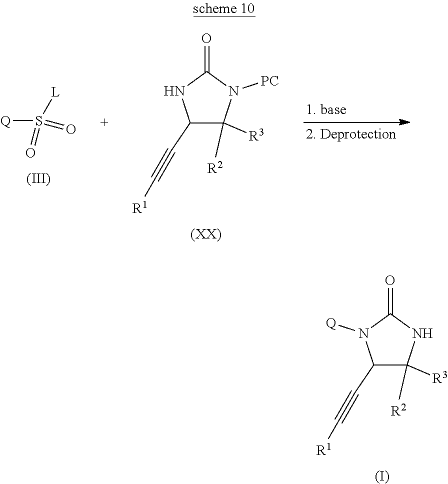

wherein R¹, R², R³, and Q are the same as defined in the above (I); L represents a lower alkyl group; and PG represents a protecting group.

Compound (I) of the present invention can be synthesized by reacting compound (XX) with compound (III) in the presence of a base such as potassium carbonate, cesium carbonate, or sodium hydride in a solvent such as THF, acetonitrile, or DMA so as to have a protected amide group. The reaction can be carried out within several minutes to several days at a temperature ranging from 0 to 150° C., and it is possible to synthesize the product preferably by reacting for several hours to 24 hours at room temperature to 100° C. Compound (I) can be obtained by deprotecting a protecting group in compound (I) in which the resulting amide group is protected under a condition generally used in organic chemistry.

Compound (XX) to be used as a raw material of Scheme 10 can be produced, for example, by the method shown in Scheme 11:

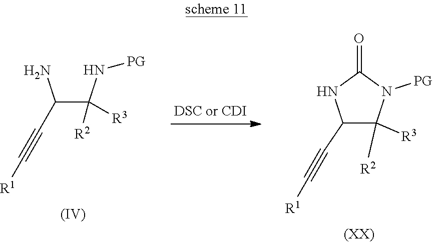

wherein R¹, R², R³ and Q are the same as defined in the above (I); and PG represents a protecting group.

Compound (XX) can be obtained by reacting compound (IV) with an imidazole-based condensing reagent such as 1,1′-carbonyldiimidazole (CDI) or a carbonate ester-based condensing reagent such as di(N-succinimidyl) carbonate (DSC) in the presence of a base such as TEA in a solvent. Although the condensing reagent can be used in an excess amount, compound (XX) can be synthesized by reaction using preferably 1 to 3 molar equivalents of CDI or DSC. Any solvent may be used as long as it is inert to the reaction. Although it is not particularly limited, for example, THF, DCM, and DMA can be used, and DCM can be preferably used. The reaction can be carried out within several minutes to several hours at a temperature ranging from 0° C. to room temperature, and it is possible to synthesize the product preferably by reacting for 30 minutes to 5 hours at room temperature.

It is possible to obtain compound (I) having the desired functional group at the desired position of the present invention by appropriately using the above methods in combination, and then carrying out a method usually used in organic synthetic chemistry (for example, an alkylation reaction, an acylation reaction, a carbamoylation reaction, and a carbamatation reaction of an amino group; alkoxylation, acylation, and carbamatation reactions of a hydroxyl group; or a reaction of inversely converting the group).

[Use of Compound (I) of the Present Invention]

The compound represented by formula (I) or a pharmaceutically acceptable salt thereof of the present invention can be prepared in the form of a conventional pharmaceutical formulation (pharmaceutical composition), which is suitable for oral administration, parenteral administration, or local administration.

Formulations for oral administration include solid formulations such as tablets, granules, powders, and capsules; and liquid formulations such as syrups. These formulations can be prepared by a conventional method. The solid formulations can be prepared by using conventional pharmaceutical carriers, for example, lactose; starches such as corn starch; crystalline celluloses such as microcrystalline cellulose; and hydroxypropyl cellulose, calcium carboxymethyl cellulose, talc, and magnesium stearate. Capsules can be prepared by encapsulating thus prepared granules or powders. Syrups can be prepared by dissolving or suspending the compound represented by formula (I) or a pharmaceutically acceptable salt thereof of the present invention in an aqueous solution containing sucrose, carboxymethyl cellulose and the like.

Formulations for parenteral administration include injections such as formulations for drip infusion. Injection formulations can also be prepared by a conventional method and can be appropriately incorporated into isotonic agents (for example, mannitol, sodium chloride, glucose, sorbitol, glycerol, xylitol, fructose, maltose, and mannose), stabilizers (for example, sodium sulfite and albumin), and antiseptics (for example, benzyl alcohol and methyl p-oxybenzoate).

The dosage of the compound represented by formula (I) or a pharmaceutically acceptable salt thereof of the present invention can vary depending on types and severity of disease; age, sex, and body weight of the patient; and dosage form, and is usually within a range from 1 mg to 1,000 mg per day for adults. The compound or a pharmaceutically acceptable salt thereof can be administered once a day, or dividedly administered twice or three times a day through an oral or parenteral route.

The compound represented by formula (I) or a pharmaceutically acceptable salt thereof of the present invention can also be used, as a DYRK inhibitor, for reagents to be used in pathological imaging and for reagents for basic experiments and research regarding the above diseases.

### EXAMPLES

The present invention will be more specifically described below by way of Examples and Test Examples, but the present invention is not limited to these Examples.

Identification of the compound was carried out by hydrogen nuclear magnetic resonance spectrum (¹H-NMR) and mass spectrum (MS). ¹H-NMR is measured at 400 MHz unless otherwise specified, and exchangeable hydrogen may not be clearly observed depending on the compound and measurement conditions. The sign “br.” means a broad signal (broad). HPLC preparative chromatography was carried out by a commercially available ODS column in a gradient mode using water/methanol or water/acetonitrile (containing formic acid) as eluents, unless otherwise specified.

### Reference Example 1

### Production of 7-(ethylsulfonyl)-[1,3]dioxolo[4′,5′:5,6]benzo[1,2-d]thiazole

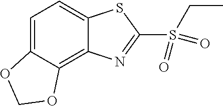 [SMILES: CCS(=O)(=O)c1nc2c3c(ccc2s1)OCO3]

(First Step)

To a solution of diisopropylamine (1.3 mL, 9.2 mmol) in THF (40 mL), n-butyllithium (1.6 M solution in hexane, 5.7 mL, 9.1 mmol) was added dropwise at −80° C. and stirred for 20 minutes. A solution of 4-bromo-1,2-(methylenedioxy)benzene (1.0 mL, 8.4 mmol) in THF (10 mL) was added dropwise to this solution and stirred for 40 minutes. This solution was stirred for 45 minutes while carbon dioxide gas was blown into it, and then stirred for another 19 hours at room temperature. The reaction solution was neutralized by addition of 2 M hydrochloric acid and extracted with ethyl acetate. The organic layer was washed with saturated brine and dried over anhydrous magnesium sulfate. After the solvent was removed under reduced pressure, the precipitated solid was collected by filtration, washed with hexane, and then dried to afford 4-bromo-3-carboxy-1,2-methylenedioxybenzene (yield: 1.1 g).

¹H NMR (DMSO-d₆) δ (ppm) 13.66 (br. s, 1H), 7.12 (d, J=8.3 Hz, 1H), 6.95 (d, J=8.3 Hz, 1H), 6.13 (s, 2H)

(Second Step)

To a solution of 4-bromo-3-carboxy-1,2-methylenedioxybenzene (1.0 g, 4.1 mmol) in dioxane (15 mL), TEA (0.63 mL, 4.5 mmol), tert-butanol (3.2 mL, 3.4 mmol), and diphenylphosphoryl azide (0.97 mL, 4.5 mmol) were added dropwise at room temperature, and the mixture was heated to reflux for 3 hours. The reaction solution was diluted with water and extracted with ethyl acetate, then the organic layer was washed with a saturated aqueous solution of sodium hydrogencarbonate, water, and saturated brine successively, and dried over anhydrous magnesium sulfate. After the solvent was removed under reduced pressure, the resultant was dried. To a solution of this intermediate in ethyl acetate (6.0 mL), 4 M hydrochloric acid in ethyl acetate solution (10 mL, 40 mmol) was added under ice cooling and the mixture was stirred for 16 hours at room temperature. The reaction solution was neutralized by addition of 2 M aqueous sodium hydroxide solution and extracted with ethyl acetate. The organic layer was washed with saturated brine and dried over anhydrous magnesium sulfate. After the solvent was removed under reduced pressure, the residue was purified by column chromatography (silica gel, hexane/ethyl acetate) to afford 3-amino-4-bromo-1,2-methylenedioxybenzene (yield: 0.80 g).

¹H NMR (DMSO-d₆) δ (ppm) 6.89 (d, J=8.4 Hz, 1H), 6.22 (d, J=8.4 Hz, 1H), 5.98 (s, 2H), 5.05 (br. s, 2H)

(Third Step)

To a solution of 3-amino-4-bromo-1,2-methylenedioxybenzene (0.79 g, 3.1 mmol) in DMF (15 mL), potassium xanthogenate (1.2 g, 7.3 mmol) was added at room temperature and stirred for 5 days at 120° C. Under ice cooling, potassium carbonate (2.2 g, mmol) and ethyl iodide (0.76 mL, 9.4 mmol) were added to this reaction solution and stirred for 2 hours at room temperature. The reaction solution was diluted with water and extracted with ethyl acetate, then the organic layer was washed with a saturated aqueous solution of sodium hydrogencarbonate, water, and saturated brine successively, and dried over anhydrous magnesium sulfate. After the solvent was removed under reduced pressure, the residue was purified by column chromatography (silica gel, hexane/ethyl acetate) to afford 7-(ethylthio)-[1,3]dioxolo[4′,5′:5,6]benzo[1,2-d]thiazole (yield: 0.26 g).

¹H NMR (DMSO-d₆) δ (ppm) 7.46 (d, J=8.4 Hz, 1H), 7.06 (d, J=8.4 Hz, 1H), 6.16 (s, 2H), 3.28-3.41 (m, 2H), 1.41 (t, J=7.3 Hz, 3H)

(Fourth Step)

To a solution of 7-(ethylthio)-[1,3]dioxolo[4′,5′:5,6]benzo[1,2-d]thiazole (0.14 g, 0.59 mmol) in DCM (2.0 mL), m-chloroperoxybenzoic acid (0.36 g, 0.48 mmol) was added under ice cooling and stirred for 1 day at room temperature. The reaction solution was diluted with water and extracted with ethyl acetate, then the organic layer was washed with saturated brine and dried over anhydrous magnesium sulfate. After the solvent was removed under reduced pressure, the residue was purified by column chromatography (silica gel, chloroform) to afford the titled compound (yield: 0.11 g).

¹H NMR (DMSO-d₆) δ (ppm) 7.81 (d, J=8.6 Hz, 1H), 7.44 (d, J=8.6 Hz, 1H), 6.30 (s, 2H), 3.70 (q, J=7.3 Hz, 2H), 1.27 (t, J=7.3 Hz, 3H)

### Reference Example 2

### Production of 4-isothiocyanato-2, 3-dihydrobenzofuran

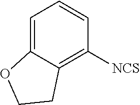

To a solution of 2,3-dihydrobenzofuran-4-amine (1.0 g, 7.41 mmol) in aqueous solution (10 mL), thiophosgene (1.41 mL, 18.52 mmol) was added at 0° C., and the mixture was stirred for 4 hours at room temperature. After completion of the reaction, the reaction mixture was diluted with water and extracted with ethyl acetate. The organic layer was washed with water and saturated brine solution and then dried over anhydrous sodium sulfate. After the solvent was concentrated under reduced pressure, the residue was purified by column chromatography (silica gel, petroleum ether) to afford the titled compound (yield: 0.9 g).

¹H NMR (CDCl₃) δ (ppm) 7.07 (t, J=8.0 Hz, 1H), 6.70 (dd, J=1.1, 7.9 Hz, 2H), 4.62 (t, J=8.8 Hz, 2H), 3.30 (t, J=8.6 Hz, 2H). GC-MS: 177.1 (M⁺).

### Reference Example 3

### Production of tert-butyl (RS)-(2-aminopent-3-yn-1-yl)carbamate

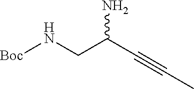

(First Step)

tert-Butyl (2,3-dihydroxypropyl)carbamate (1.5 g, 7.8 mmol) was dissolved in water (13 mL), and the reaction vessel was shielded with aluminum foil. Then, sodium periodate (2 g, 9.4 mmol) was added thereto. After the mixture solution was stirred for 2 hours at room temperature, the precipitated product was filtered, and the filtrate was extracted with chloroform. The organic layer was dried over anhydrous sodium sulfate and the solvent was removed under reduced pressure to afford an aldehyde intermediate. This was immediately dissolved in THF (15 mL) and added to 1-propynylmagnesium bromide in 0.5 M-THF solution (31 mL, 16 mmol), which was cooled to −80° C. After the reaction liquid was stirred for 30 minutes at −80° C. and for another 30 minutes at 0° C., aqueous ammonium chloride solution was added thereto to quench the reaction, and the mixture was extracted with ethyl acetate. The organic layer was dried over anhydrous sodium sulfate, and the solvent was removed to afford tert-butyl (RS)-(2-hydroxypent-3-yn-1-yl)carbamate (yield: 1.4 g).

¹H NMR (CDCl₃) δ (ppm) 4.93-4.98 (m, 1H), 4.38-4.43 (m, 1H), 3.40-3.45 (m, 1H), 3.20-3.30 (m, 1H), 2.59 (s, 1H), 1.85 (d, J=2.1 Hz, 3H), 1.41-1.54 (m, 9H)

(Second Step)

To a solution of tert-butyl (RS)-(2-hydroxypent-3-yn-1-yl)carbamate (1.3 g, 6.3 mmol) in THF (25 mL), phthalimide (1.0 g, 7.0 mmol) and triphenylphosphine (2.2 g, 8.2 mmol) were added, and a solution of diethyl azodicarboxylate in 2.2 M toluene (3.5 mL, 7.6 mmol) was then added slowly at room temperature. After the mixture solution was stirred for 17 hours, the solvent was removed to afford a crude product of the intermediate. This intermediate was dissolved in a THF-methanol mixed solvent (1:1, 24 mL), then hydrazine monohydrate (4.8 g, 95 mmol) was added thereto and stirred for 3 hours at 50° C. The reaction liquid was filtered by celite, and the filtrate was concentrated under reduced pressure. The residue was diluted with ethyl acetate, washed with 1 M aqueous sodium hydroxide solution, and then extracted with 1 M hydrochloric acid. The resulting acidic extract was made alkaline with 2 M aqueous sodium hydroxide solution and extracted with ethyl acetate. The organic layer was dried over anhydrous sodium sulfate, and the solvent was removed to afford the titled compound (yield: 0.42 g).

¹H NMR (CDCl₃) δ (ppm) 4.96 (s, 1H), 3.71-3.76 (m, 1H), 3.57-3.66 (m, 1H), 3.23-3.33 (m, 1H), 3.08-3.20 (m, 1H), 1.81 (d, J=2.2 Hz, 3H), 1.45 (s, 9H)

### Reference Example 4

### Production of 2-(ethylsulfonyl)-7,8-dihydro-[1,4]dioxyno[2′,3′:5,6]benzo[1,2-d]thiazole

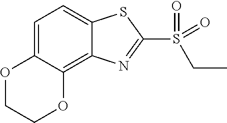 [SMILES: CCS(=O)(=O)c1nc2c3c(ccc2s1)OCCO3]

(First Step)

To a solution of 7-(ethylthio)-[1,3]dioxolo[4′,5′:5,6]benzo[1,2-d]thiazole (239 mg, 1.0 mmol) in chloroform (2 mL), boron tribromide (1 M-DCM solution, 1.5 mL, 1.5 mmol) was added at room temperature and stirred for 17 hours. Methanol was added to the reaction solution, concentrated, and then azeotroped with ethanol. Ethanol was added to the residue, and the precipitated solid was collected by filtration, washed with ethanol, and then dried to afford 2-(ethylthio)benzo[d]thiazole-4,5-diol (yield: 252 mg).

¹H-NMR (DMSO-d₆) δ: 7.17 (d, J=8.5 Hz, 1H), 6.85 (d, J=8.5 Hz, 1H), 5.89 (br s, 2H), 3.31 (q, J=7.3 Hz, 2H), 1.40 (t, J=7.3 Hz, 3H).

(Second Step)

To a solution of 2-(ethylthio)benzo[d]thiazole-4,5-diol (252 mg, 1 mmol) in DMF (3 mL), cesium carbonate (652 mg, 2.0 mmol) and 1,2-dibromoethane (0.172 mL, 2.0 mmol) were added at room temperature and stirred for 2 hours at 70° C. Water and saturated sodium bicarbonate solution were added to the reaction solution, the mixture was extracted with ethyl acetate, then the organic layer was washed with saturated brine and dried over anhydrous magnesium sulfate. After the solvent was removed under reduced pressure, the residue was purified by column chromatography (silica gel, hexane/ethyl acetate) to afford 2-(ethylthio)-7,8-dihydro-[1,4]dioxyno[2′,3′:5,6]benzo[1,2-d]thiazole (yield: 0.14 g).

¹H-NMR (CDCl₃) δ: 7.16 (d, J=8.5 Hz, 1H), 6.89 (d, J=8.5 Hz, 1H), 4.46-4.42 (m, 2H), 4.34-4.30 (m, 2H), 3.33 (q, J=7.5 Hz, 2H), 1.46 (t, J=7.5 Hz, 3H).

(Third Step)

To a solution of 2-(ethylthio)-7,8-dihydro-[1,4]dioxyno[2′,3′:5,6]benzo[1,2-d]thiazole (0.14 g, 0.56 mmol) in chloroform (5.0 mL), m-chloroperoxybenzoic acid (0.41 g, 1.67 mmol) was added under ice cooling and stirred for 66 hours at room temperature. Saturated sodium bicarbonate solution and a saturated aqueous solution of thiosodium sulfate were added to the reaction solution, and the mixture was extracted with ethyl acetate, then the organic layer was washed with a mixed solution of the saturated sodium bicarbonate solution and the saturated aqueous solution of thiosodium sulfate and dried over anhydrous magnesium sulfate. After the solvent was removed under reduced pressure, the residue was purified by column chromatography (silica gel, hexane/ethyl acetate) to afford the titled compound (yield: 0.10 g).

¹H-NMR (CDCl₃) δ: 7.40 (d, J=9.1 Hz, 1H), 7.18 (d, J=9.1 Hz, 1H), 4.49-4.47 (m, 2H), 4.38-4.36 (m, 2H), 3.55 (q, J=7.5 Hz, 2H), 1.40 (t, J=7.5 Hz, 3H).

### Reference Example 5

### Production of tert-butyl (RS)-2-oxo-4-(prop-1-yn-1-yl)imidazolidine-1-carboxylate

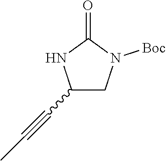 [SMILES: CC#CC1CN(C(=O)OC(C)(C)C)C(=O)N1]

To a suspension of N,N′-disuccinimidyl carbonate (194 mg, 0.76 mmol) in chloroform (1 mL), triethylamine (0.14 mL, 1.51 mmol) was added under ice cooling, and then a solution of tert-butyl (RS)-(2-aminopent-3-yn-1-yl)carbamate (100 mg, 0.504 mmol) in chloroform (2 mL) was dropwise added during a period of 40 minutes. After the mixture was stirred for 2 hours at room temperature, water was added thereto. After being extracted with chloroform, the organic layer was dried over anhydrous magnesium sulfate. After the solvent was removed under reduced pressure, the residue was purified by column chromatography (silica gel, hexane/ethyl acetate) to afford the titled compound (yield: 0.84 g).

¹H-NMR (CDCl₃) δ: 4.87 (s, 1H), 4.33-4.29 (m, 1H), 3.99 (dd, J=8.5, 10.4 Hz, 1H), 3.77 (dd, J=5.5, 10.4 Hz, 1H), 1.81 (d, J=1.8 Hz, 3H), 1.51 (s, 9H).

### Example 1

### Production of (RS)-1-([1,3]dioxolo[4′,5′:5,6]benzo[1,2-d]thiazol-7-yl)-5-(prop-1-yn-1-yl)imidazolidin-2-one [Formula 21]

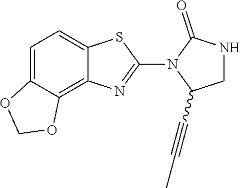 [SMILES: CC#CC1CNC(=O)N1c1nc2c3c(ccc2s1)OCO3]

(First Step)

A mixture of 7-(ethylsulfonyl)-[1,3]dioxolo[4′,5′:5,6]benzo[1,2-d]thiazole (100 mg, 0.369 mmol) and tert-butyl (RS)-(2-aminopent-3-yn-1-yl)carbamate (292 mg, 1.474 mmol) was stirred for 6 hours at 100° C. The reaction mixture was diluted with ethyl acetate and washed with 1 M hydrochloric acid. The organic layer was dried over anhydrous sodium sulfate, and to the residue obtained by removing the solvent, 4 M hydrochloric acid-dioxane solution (3 mL) was added and stirred for 30 minutes at room temperature. The reaction liquid was made alkaline by addition of aqueous sodium hydroxide solution and extracted with ethyl acetate. The organic layer was washed with water and saturated brine and then dried over anhydrous sodium sulfate. The solvent was removed under reduced pressure to afford (RS)—N2-([1,3]dioxolo[4′,5′:5,6]benzo[1,2-d]thiazol-7-yl)pent-3-yn-1,2-diamine (yield: 40 mg).

¹H NMR (CDCl₃) δ (ppm) 7.03 (d, J=8.2 Hz, 1H), 6.70 (d, J=8.2 Hz, 1H), 6.05 (s, 2H), 4.69 (s, 1H), 3.61-3.69 (m, 1H), 3.06 (dd, J=12.7, 4.6 Hz, 1H), 2.98 (dd, J=12.7, 5.2 Hz, 1H), 1.84 (d, J=2.2 Hz, 3H)

(Second Step)

To a solution of (RS)—N2-([1,3]dioxolo[4′,5′:5,6]benzo[1,2-d]thiazol-7-yl)pent-3-yn-1,2-diamine (40 mg, 0.15 mmol) in DMF (2.0 mL), N,N′-disuccinimidyl carbonate (0.11 g, 0.44 mmol) was added. After the mixture was stirred for 30 minutes at room temperature, water was added to the reaction liquid and extracted with ethyl acetate. The organic layer was washed with water and saturated brine and dried over anhydrous sodium sulfate. The solvent was removed under reduced pressure, and the residue was purified by column chromatography (aminesilica gel, hexane/ethyl acetate) to afford the titled compound (yield: 18 mg).

¹H NMR (DMSO-d₆) δ (ppm) 8.05 (s, 1H), 7.38 (d, J=8.3 Hz, 1H), 6.95 (d, J=8.3 Hz, 1H), 6.07-6.16 (m, 2H), 5.29-5.37 (m, 1H), 3.84 (dd, J=9.0 Hz, 1H), 3.25-3.48 (m, 1H), 1.80 (d, J=2.1 Hz, 3H); LCMS (m/z): 302.1 [M+H]⁺.

### Example 2

### Production of (R)-1-([1,3]dioxolo[4′,5′:5,6]benzo[1,2-d]thiazol-7-yl)-5-(1-propyn-1-yl)imidazolidin-2-one

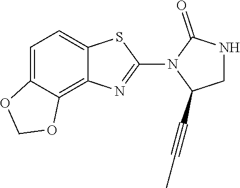 [SMILES: CC#C[C@@H]1CNC(=O)N1c1nc2c3c(ccc2s1)OCO3]

(RS)-1-([1,3]dioxolo[4′,5′:5,6]benzo[1,2-d]thiazol-7-yl)-5-(propynyl)imidazolidin-2-one (0.15 g, 0.49 mmol) produced in Example 1 was purified by using supercritical fluid chromatography (Chiralpak IG (30×250 mm), carbon dioxide/methanol) to afford the titled compound (yield: 54 mg) as a fraction that eluted first (retention time: 2.37 minutes).

¹H NMR (500 MHz, DMSO-d₆) δ (ppm) 8.04 (br. s, 1H), 7.38 (d, J=8.2 Hz, 1H), 6.94 (d, J=8.2 Hz, 1H), 6.12 (d, J=4.9 Hz, 2H), 5.28-5.36 (m, 1H), 3.83 (t, J=9.0 Hz, 1H), 3.40-3.48 (m, 1H), 1.80 (d, J=2.0 Hz, 3H); LCMS (m/z): 302.3 [M+H]⁺.

### Example 3

### Production of (S)-1-([1,3]dioxolo[4′,5′:5,6]benzo[1,2-d]thiazol-7-yl)-5-(1-propyn-1-yl)imidazolidin-2-one

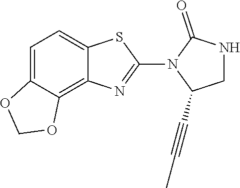 [SMILES: CC#C[C@H]1CNC(=O)N1c1nc2c3c(ccc2s1)OCO3]

In the same manner as Example 2, (RS)-1-([1,3]dioxolo[4′,5′:5,6]benzo[1,2-d]thiazol-7-yl)-5-(propynyl)imidazolidin-2-one (Example 1) (0.15 g, 0.49 mmol) was purified by using supercritical fluid chromatography (Chiralpak IG (30×250 mm), carbon dioxide/methanol) to afford a fraction eluted after the R-body (retention time: 2.37 minutes) of Example 2 (retention time: 4.19 minutes) as the titled compound having the opposite steric configuration (yield: 40 mg).

¹H NMR (500 MHz, DMSO-d₆) δ (ppm) 8.04 (br. s, 1H), 7.38 (d, J=8.2 Hz, 1H), 6.94 (d, J=8.2 Hz, 1H), 6.12 (d, J=4.9 Hz, 2H), 5.28-5.36 (m, 1H), 3.83 (t, J=9.0 Hz, 1H), 3.40-3.48 (m, 1H), 1.80 (d, J=2.0 Hz, 3H); LCMS (m/z): 302.3 [M+H]⁺.

### Example 4

### Production of (4S,5S)-1-(7,8-dihydrobenzofuro[4,5-d]thiazol-2-yl)-4-methyl-5-(prop-1-yn-1-yl)imidazolidin-2-one

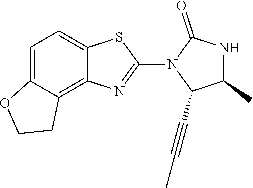 [SMILES: CC#C[C@H]1[C@H](C)NC(=O)N1c1nc2c3c(ccc2s1)OCC3]

(First Step)

To a solution of tert-butyl (S)-{1-[methoxy(methyl)amino]-1-oxopropan-2-yl}carbamate (5.0 g, 21.55 mmol) in THF (50 mL) which was cooled to −75° C., 1-propynylmagnesium bromide in 0.5 M-THF solution (128.31 mL, 64.65 mmol) was dropwise added and stirred for 16 hours at room temperature. Water was added to the reaction liquid and extracted with ethyl acetate. The organic layer was washed with water and saturated brine and dried over anhydrous sodium sulfate. The solvent was removed under reduced pressure to afford tert-butyl (S)-(3-oxohex yn-2-yl)carbamate (yield: 4.4 g).

¹H NMR (DMSO-d₆) δ (ppm) 7.35 (d, J=7.2 Hz, 1H), 4.04-3.89 (m, 1H), 2.06 (s, 3H), 1.39 (s, 9H), 1.20 (d, J=7.3 Hz, 3H). LCMS (m/z): 212.40 [M+H]⁺.

(Second Step)

To a solution of tert-butyl (S)-(3-oxohex-4-yn-2-yl)carbamate (3.9 g, 18.48 mmol) in methanol (50 mL), cerium(III) chloride heptahydrate (8.93 g, 24.03 mmol) and sodium borohydride (0.913 g, 24.03 mmol) were added under ice cooling and stirred for 1 hour at room temperature. Water was added to the reaction liquid and extracted with ethyl acetate. The organic layer was washed with water and saturated brine and dried over anhydrous sodium sulfate. The solvent was removed under reduced pressure to afford partially purified tert-butyl ((2S,3RS)-3-hydroxyhex-4-yn-2-yl)carbamate (yield: 4.1 g).

¹H NMR (DMSO-d₆) δ (ppm) 6.52-6.47 (m, 1H), 5.26 (dd, J=3.2, 5.9 Hz, 1H), 4.15-4.09 (m, 1H), 3.50 (br. s, 1H), 1.82-1.76 (m, 3H), 1.40-1.35 (m, 9H), 1.05-1.01 (m, 3H). LCMS (m/z): 214.24 [M+H]⁺.

(Third Step)

To a solution of tert-butyl ((2S,3RS)-3-hydroxyhex-4-yn-2-yl)carbamate (4.1 g, 19.25 mmol) in THF (50 mL), phthalimide (2.26 g, 15.40 mmol) and triphenylphosphine (5.54 g, 21.17 mmol) were added under ice cooling, and diethyl azodicarboxylate (3.67 g, 21.17 mmol) was then added slowly, and the mixture solution was stirred for 17 hours at room temperature. Water was added to the reaction liquid and extracted with ethyl acetate. The organic layer was washed with water and saturated brine and dried over anhydrous sodium sulfate. The solvent was removed under reduced pressure, and the residue was purified by column chromatography (silica gel, hexane/ethyl acetate) to afford tert-butyl [(2S,3RS)-3-(1,3-dioxoisoindolin-2-yl)hex-4-yn-2-yl]carbamate (yield: 3.0 g).

¹H NMR (DMSO-d₆) δ (ppm) 7.94-7.80 (m, 4H), 6.88 (d, J=7.6 Hz, 1H), 4.65 (dd, J=2.4, 9.8 Hz, 1H), 4.20-4.09 (m, 1H), 1.81 (d, J=2.4 Hz, 3H), 1.28 (d, J=6.8 Hz, 3H), 0.97 (s, 9H). LCMS (m/z): 343.43 [M+H]′.

(Fourth Step)

tert-butyl [(2S,3RS)-3-(1,3-dioxoisoindolin-2-yl)hex-4-yn-2-yl]carbamate (3.0 g, 8.77 mmol) was dissolved in a THF-methanol mixed solvent (1:1, 60 mL), and hydrazine monohydrate (4.38 g, 87.72 mmol) was then added thereto and stirred for 3 hours at 80° C. Water was added to the reaction liquid and extracted with ethyl acetate. The organic layer was washed with water and saturated brine and dried over anhydrous sodium sulfate. The solvent was removed under reduced pressure to afford tert-butyl ((2S,3RS)-3-aminohex-4-yn-2-yl)carbamate (yield: 1.6 g).

LCMS (m/z): 213.19 [M+H]⁺.

(Fifth Step)

To a solution of 4-isothiocyanato-2, 3-dihydrobenzofuran (0.85 g, 4.80 mmol) in ethanol (15 mL), TEA (2.02 mL, 14.41 mmol) and tert-butyl ((2S,3RS)-3-aminohex-4-yn-2-yl)carbamate (1.32 g, 6.24 mmol) were added and stirred for 16 hours at room temperature. Water was added to the reaction liquid, the mixture was extracted with ethyl acetate, and the organic layer was washed with water and saturated brine and dried over anhydrous sodium sulfate. The solvent was removed under reduced pressure, and the residue was purified by column chromatography (silica gel, petroleum ether/ethyl acetate) to afford tert-butyl {(2S,3RS)-3-[3-(2, 3-dihydrobenzofuran-4-yl)thioureido]hex-4-yn-2-yl}carbamate (yield: 1.5 g).

¹H NMR (DMSO-d₆) δ (ppm) 9.28 (br. s, 1H), 7.85 (d, J=8.8 Hz, 1H), 7.10-6.98 (m, 2H), 6.77 (br. s, 1H), 6.59 (d, J=8.3 Hz, 1H), 5.32-5.22 (m, 1H), 4.54-4.48 (m, 2H), 3.74-3.69 (m, 1H), 3.20-3.00 (m, 2H), 1.81 (d, J=2.0 Hz, 3H), 1.39 (s, 9H), 1.13 (d, J=6.6 Hz, 3H). LCMS (m/z): 390.55 [M+H]⁺.

(Sixth Step)

To a solution of tert-butyl {(2S,3RS)-3-[3-(2,3-dihydrobenzofuran-4-yl)thioureido]hex-4-yn-2-yl}carbamate (1.4 g, 3.60 mmol) in chloroform (15 mL), sodium carbonate (3.023 mg, 35.99 mmol) and benzyltrimethylammonium tribromide (1.11 g, 2.88 mmol) were added under ice cooling and stirred for 1 hour at room temperature. Water was added to the reaction liquid, the mixture was extracted with DCM, and the organic layer was washed with water and saturated brine and dried over anhydrous sodium sulfate. The solvent was removed under reduced pressure, and the residue was purified by column chromatography (silica gel, petroleum ether/ethyl acetate) to afford tert-butyl {(2S,3RS)-3-[(7,8-dihydrobenzofuro[4,5-d]thiazol-2-yl)amino]hex-4-yn-2-yl}carbamate (yield: 0.9 g).

¹H NMR (DMSO-d₆) δ (ppm) 8.19 (d, J=8.4 Hz, 1H), 7.36 (d, J=4.4, 8.0 Hz, 1H), 6.77 (d, J=8.4 Hz, 1H), 6.52 (dd, J=2.8, 8.4 Hz, 1H), 4.72-4.68 (m, 1H), 4.55 (t, J=8.8 Hz, 2H), 3.79-3.71 (m, 1H), 3.28-3.24 (m, 2H), 1.79 (s, 3H), 1.37 (s, 9H), 1.19-1.15 (m, 3H). LCMS (m/z): 388.35 [M+H]⁺.

(Seventh Step)

To a solution of tert-butyl {(2S,3RS)-3-[(7,8-dihydrobenzofuro[4,5-d]thiazol-2-yl)amino]hex-4-yn-2-yl}carbamate (0.9 g, 2.32 mmol) in ethyl acetate (20 mL), 4 M hydrochloric acid-ethyl acetate solution (5 mL) was added under ice cooling and stirred for 4 hours at room temperature. The solvent was removed under reduced pressure to afford (2S,3RS)—N3-(7,8-dihydrobenzofuro[4,5-d]thiazol-2-yl)hex-4-yn-2,3-diamine hydrochloride (yield: 0.9 g) as a crude purified product.

LCMS (m/z): 288.23 [M+H]⁺.

(Eighth Step)

To a solution of (2S,3RS)—N3-(7,8-dihydrobenzofuro[4,5-d]thiazol-2-yl)hex-4-yn-2,3-diamine hydrochloride (0.8 g, 2.79 mmol) in DMF (8 mL), TEA (3.91 mL, 27.87 mmol) and N,N′-disuccinimidyl carbonate (0.713 g, 2.79 mmol) were added under ice cooling. After the mixture was stirred for 3 hours at room temperature, water was added to the reaction liquid and extracted with ethyl acetate. The organic layer was washed with water and saturated brine and dried over anhydrous sodium sulfate. The solvent was removed under reduced pressure, and the residue was purified by column chromatography (silica gel, petroleum ether/ethyl acetate) to afford (4S,5RS)-1-(7,8-dihydrobenzofuro[4,5-d]thiazol-2-yl)-4-methyl-5-(prop-1-yn yl)imidazolidin-2-one (yield: 0.43 g) as a diastereomeric mixture. The resulting diastereomeric mixture was purified by using supercritical fluid chromatography (Chiralpak IC (30×250 mm), carbon dioxide/methanol) to afford the titled compound (yield: 248 mg) as a fraction eluted first (retention time: 2.527 minutes).

¹H NMR (500 MHz, DMSO-d₆) δ (ppm) 8.18 (br. s, 1H), 7.60 (d, J=8.5 Hz, 1H), 6.76 (d, J=8.5 Hz, 1H), 4.89 (t, J=2.5 Hz, 1H), 4.62 (t, J=9.5 Hz, 2H), 3.82-3.72 (m, 1H), 3.38 (t, J=9.0 Hz, 2H), 1.80 (d, J=2.0 Hz, 3H), 1.24 (d, J=6.0 Hz, 3H). LCMS (m/z): 314.28 [M+H]⁺.

### Example 5

### Production of (4S,5R)-1-(7,8-dihydrobenzofuro[4,5-d]thiazol-2-yl)-4-methyl-5-(prop-1-yn-1-yl)imidazolidin-2-one

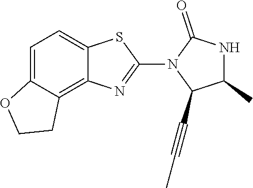 [SMILES: CC#C[C@@H]1[C@H](C)NC(=O)N1c1nc2c3c(ccc2s1)OCC3]

The diastereomeric mixture obtained in the eighth step of Example 4 was purified by using supercritical fluid chromatography (Chiralpak IC (30×250 mm), carbon dioxide/methanol) to afford (4S,5R)-1-(7,8-dihydrobenzofuro[4,5-d]thiazol-2-yl)-4-methyl-5-(prop-1-yn-1-yl)imidazolidin-2-one (titled compound) (yield: 30 mg) as a fraction eluted after (4S,5S)-1-(7,8-dihydrobenzofuro[4,5-d]thiazol-2-yl)-4-methyl-5-(prop-1-yn-1-yl)imidazolidin-2-one (retention time: 2.527 minutes) (retention time: 4.450 minutes).

¹H NMR (DMSO-d₆) δ (ppm) 8.00 (br. s, 1h), 7.60 (d, J=8.3 Hz, 1H), 6.76 (d, J=8.3 Hz, 1H), 5.35 (d, J=5.9 Hz, 1H), 4.62 (t, J=8.7 Hz, 2H), 4.10-4.00 (m, 1H), 3.38 (t, J=8.7 Hz, 2H), 1.81 (d, J=1.0 Hz, 3H), 1.29 (d, J=6.1 Hz, 3H). LCMS (m/z): 314.19 [M+H]⁺.

### Example 6

### Production of (RS)-1-(imidazo[1,2-a]thiazolo[5,4-e]pyridin-2-yl)-5-(prop-1-yn-1-yl)imidazolidin-2-one

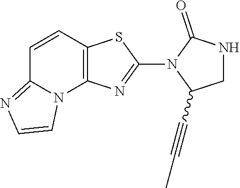 [SMILES: CC#CC1CNC(=O)N1c1nc2c(ccc3nccn32)s1]

(First Step)

Under ice cooling, imidazo[1,2-a]pyridin-5-amine (1.99 g, 14.95 mmol) was suspended in a THF-DCM mixed solvent (1:1, 266 mL), and 1,1′-dithiocarbonyldiimidazole (8.88 g, 44.8 mmol) was added thereto and stirred for 18 hours at room temperature. The reaction mixture was filtered, the solid was washed with hexane and then dried to afford N-(imidazo[1,2-a]pyridin-5-yl)-1H-imidazole-1-carbothioamide (yield: 2.0 g).

¹H NMR (DMSO-d₆) δ (ppm) 8.74-8.70 (m, 1H), 8.34 (dd, J=7.9, 1.0 Hz, 1H), 8.24 (dd, J=2.1, 0.8 Hz, 1H), 8.09-8.06 (m, 1H), 7.96 (d, J=2.1 Hz, 1H), 7.81-7.74 (m, 1H), 7.37-7.31 (m, 1H), 7.25-7.21 (m, 1H), 6.98-6.94 (m, 1H).

(Second Step)

To a solution of tert-butyl (RS)-(2-aminopent-3-yn-1-yl)carbamate (300 mg, 1.51 mmol) in THF (15 mL), N-(imidazo[1,2-a]pyridin-5-yl)-1H-imidazole-1-carbothioamide (368 mg, 1.51 mmol) was added. After the reaction mixture was stirred for 30 minutes at room temperature, water was added to the reaction liquid and extracted with ethyl acetate. The organic layer was washed with water and saturated brine and dried over anhydrous sodium sulfate. The solvent was removed under reduced pressure, and the residue was purified by column chromatography (aminesilica gel, hexane/ethyl acetate) to afford tert-butyl (RS)-{2-[3-(imidazo[1,2-a]pyridin-5-yl)thioureido]pent-3-yn-1-yl}carbamate (yield: 142 mg).

LCMS (m/z): 374.3 [M+H]⁺.

(Third Step)

To a solution of tert-butyl (RS)-{2-[3-(imidazo[1,2-a]pyridin-5-yl)thioureido]pent-3-yn-1-yl}carbamate (142 mg, 0.38 mmol) in acetonitrile (15 mL), a solution of bromine (17.63 μL, 0.342 mmol) in acetic acid (76 μL) was added. After the reaction mixture was stirred for 30 minutes at room temperature, the solvent was removed under reduced pressure, ethyl acetate was added thereto, and the mixture was washed with 2 M aqueous sodium hydroxide solution, water, and saturated brine and dried over anhydrous sodium sulfate. The solvent was removed under reduced pressure, and the residue was purified by column chromatography (silica gel, hexane/ethyl acetate) to afford tert-butyl (RS)-[2-(imidazo[1,2-a]thiazolo[5,4-e]pyridin-2-ylamino)pent-3-yn-1-yl]carbamate (yield: 43 mg).

LCMS (m/z): 372.3 [M+H]⁺.

(Fourth Step)

To a solution of tert-butyl (RS)-[2-(imidazo[1,2-a]thiazolo[5,4-e]pyridin-2-ylamino)pent-3-yn-1-yl]carbamate (43 mg, 0.116 mmol) in ethyl acetate (5.0 mL), 4 M hydrochloric acid-ethyl acetate solution (2 mL) was added and stirred for 16 hours at room temperature. To a solution of the residue obtained by removing the solvent under reduced pressure in DMF (1.15 mL), TEA (161 μL, 1.158 mmol) and N,N′-disuccinimidyl carbonate (2.97 mg, 0.116 mmol) were added. After the mixture was stirred for 3 hours at room temperature, water was added to the reaction liquid and extracted with ethyl acetate. The organic layer was washed with water and saturated brine and dried over anhydrous sodium sulfate. The solvent was removed under reduced pressure to afford the titled compound (yield: 21 mg).

¹H NMR (DMSO-d₆) δ (ppm) 8.21 (s, 1H), 8.12-8.06 (m, 1H), 7.84 (d, J=9.34 Hz, 1H), 7.66 (d, J=1.28 Hz, 1H), 7.47 (dd, J=0.76, 9.29 Hz, 1H), 5.42 (dp, J=2.19, 8.98 Hz, 1H), 3.89 (t, J=9.07 Hz, 1H), 3.48 (ddd, J=1.06, 2.80, 9.18 Hz, 1H), 1.80 (d, J=2.11 Hz, 3H). LCMS (m/z): 298.1 [M+H]⁺.

### Example 7

### Production of (RS)-5-(prop-1-yn-1-yl)-1-(thiazolo[5′,4′:5,6]benzo[1,2-d]oxazole-7-yl)imidazolidin-2-one

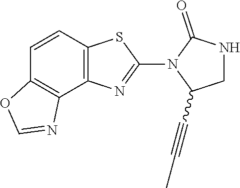 [SMILES: CC#CC1CNC(=O)N1c1nc2c(ccc3ocnc32)s1]

(First Step)

To a solution of benzo[d]oxazole-4-amine (0.5 g, 3.70 mmol) in THF (10 mL), 1,1′-dithiocarbonyldiimidazole (0.96 g, 4.81 mmol) was gradually added under ice cooling and stirred for 3 hours at room temperature, and water (0.1 mL) was added thereto and stirred for another 15 minutes. A solution of tert-butyl (RS)-(2-aminopent-3-yn-1-yl)carbamate (0.88 g, 4.44 mmol) in THF (5 mL) was added to the reaction mixture solution and stirred for 2 hours at room temperature. Water was added to the reaction liquid and extracted with ethyl acetate. The organic layer was washed with water and saturated brine and dried over anhydrous sodium sulfate. The solvent was removed under reduced pressure to afford tert-butyl (RS)-{2-[3-(benzo[d]oxazole-4-yl)thioureido]pent-3-yn-1-yl}carbamate (yield: 0.5 g).

LCMS (m/z): 375.29 [M+H]⁺.

(Second Step)

To a solution of tert-butyl (RS)-{2-[3-(benzo[d]oxazole-4-yl)thioureido]pent-3-yn-1-yl}carbamate (0.5 g, 1.33 mmol) in DCM (10 mL), benzyltrimethylammonium tribromide (0.42 g, 1.07 mmol) was added under ice cooling and stirred for 1 hour at room temperature. Water was added to the reaction liquid, the mixture was extracted with DCM, and the organic layer was washed with water and saturated brine and dried over anhydrous sodium sulfate. The solvent was removed under reduced pressure, and the residue was purified by column chromatography (silica gel, petroleum ether/ethyl acetate) to afford tert-butyl (RS)-[2-(thiazolo[5′,4′:5,6]benzo[1,2-d]oxazole-7-ylamino)pent-3-yn-1-yl]carbamate carbamate (yield: 0.26 g).

LCMS (m/z): 373.52 [M+H]⁺.

(Third Step)

To a solution of tert-butyl (RS)-[2-(thiazolo[5′,4′:5,6]benzo[1,2-d]oxazole-7-ylamino)pent-3-yn yl] carbamate (0.06 g, 0.163 mmol) in DCM (3 mL), 4 M hydrochloric acid-dioxane solution (1 mL) was added under ice cooling and stirred for 2 hours at room temperature. The solvent was removed under reduced pressure to afford (RS)—N2-(thiazolo[5′,4′:5,6]benzo[1,2-d]oxazole-7-yl)pent-3-yn-1,2-diamine hydrochloride (yield: 0.05 g) as a partially purified product.

(Fourth Step)

To a solution of (RS)—N2-(thiazolo[5′,4′:5,6]benzo[1,2-d]oxazole-7-yl)pent-3-yn-1,2-diamine hydrochloride (0.18 g, 0.58 mmol) in DMF (5 mL), TEA (0.4 mL, 2.92 mmol) and N,N′-disuccinimidyl carbonate (0.165 g, 0.64 mmol) were added under ice cooling. After the mixture was stirred for 16 hours at room temperature, water was added to the reaction liquid and extracted with ethyl acetate. The organic layer was washed with water and saturated brine and dried over anhydrous sodium sulfate. The solvent was removed under reduced pressure, and then the resulting residue was purified using HPLC preparative system to afford the titled compound (yield: 15 mg).

¹H NMR (500 MHz, DMSO-d₆) δ (ppm) 8.81 (s, 1H), 7.98 (d, J=9.0 Hz, 1H), 7.90 (br. s, 1H), 7.71 (d, J=8.5 Hz, 1H), 5.46-5.44 (m, 1H), 3.87 (t, J=9.0 Hz, 1H), 3.47 (dd, J=2.5, 9.0 Hz, 1H), 1.79 (s, 3H). LCMS (m/z): 299.17 [M+H]⁺.

Each of the compounds of Examples 8 to 22, 24 to 54, and 56 to 84 in the following [Table 1] to [Table 3] were produced with corresponding raw materials (commercially available products or compounds derivatized from commercially available compounds by a known method or an equivalent method thereof) according to the method described in the above Examples, if necessary, by using a method which is usually used in organic synthetic chemistry in combination. In addition, compounds with chiral centers were produced using chiral starting materials, asymmetric synthesis, preparative purification by chiral columns, or a combination thereof. The physicochemical data of each compound is shown in [Table 4] and [Table 5].

In the tables, when the bond of the substituent at an optically active center is indicated by a wavy line, it represents a racemic body; and when the bond of the substituent at an optically active center is indicated by a solid line, each represents an enantiomer for a substitution position thereof and a compound obtained as a single compound by optical resolution. The analytical conditions and retention times of the stereoisomers obtained by optical resolution were also shown in [Table 6].

TABLE 1

| Example | | |
|---|---|---|
| Number | Structure | Compound Name |
| 8 | 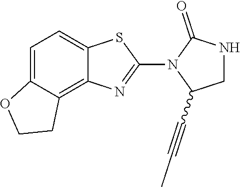 [SMILES: CC#CC1CNC(=O)N1c1nc2c3c(ccc2s1)OCC3] | (RS)-1-(7,8-dihydrobenzofuro[4,5- d]thiazol-2-yl)-5-(prop-1-yn-1- yl)imidazolidin-2-one |
| 9 (Enantiomer A of 8) | 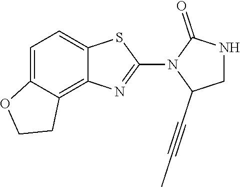 [SMILES: CC#CC1CNC(=O)N1c1nc2c3c(ccc2s1)OCC3] | 1-(7,8-dihydrobenzofuro[4,5-d]thiazol-2- yl)-5-(prop-1-yn-1-yl)imidazolidin-2-one |
| 10 (Enantiomer B of 8) | 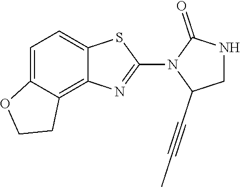 [SMILES: CC#CC1CNC(=O)N1c1nc2c3c(ccc2s1)OCC3] | 1-(7,8-dihydrobenzofuro[4,5-d]thiazol-2- yl)-5-(prop-1-yn-1-yl)imidazolidin-2-one |
| 11 | 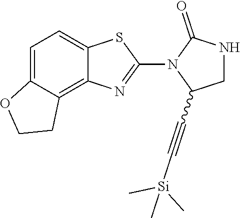 [SMILES: C[Si](C)(C)C#CC1CNC(=O)N1c1nc2c3c(ccc2s1)OCC3] | (RS)-1-(7,8-dihydrobenzofuro[4,5- d]thiazol-2-yl)-5- [(trimethylsilyl)ethynyl]imidazolidin-2- one |
| 12 | 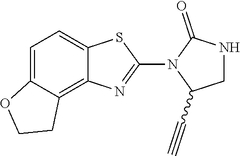 [SMILES: C#CC1CNC(=O)N1c1nc2c3c(ccc2s1)OCC3] | (RS)-1-(7,8-dihydrobenzofuro[4,5- d]thiazol-2-yl)-5-ethynylimidazolidin-2- on |
| 13 (Enantiomer A of 12) | 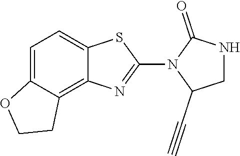 [SMILES: C#CC1CNC(=O)N1c1nc2c3c(ccc2s1)OCC3] | 1-(7,8-dihydrobenzofuro[4,5-d]thiazol-2- yl)-5-ethynylimidazolidin-2-one |
| 14 (Enantiomer B of 12) | 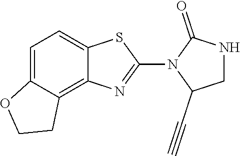 [SMILES: C#CC1CNC(=O)N1c1nc2c3c(ccc2s1)OCC3] | 1-(7,8-dihydrobenzofuro[4,5-d]thiazol-2- yl)-5-ethynylimidazolidin-2-one |
| 15 | 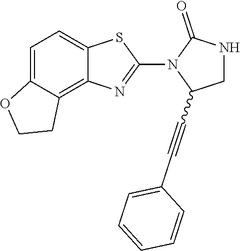 [SMILES: O=C1NCC(C#Cc2ccccc2)N1c1nc2c3c(ccc2s1)OCC3] | (RS)-1-(7,8-dihydrobenzofuro[4,5- d]thiazol-2-yl)-5- (phenylethynyl)imidazolidin-2-one |
| 16 | 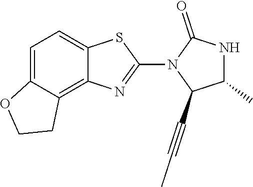 [SMILES: CC#C[C@@H]1[C@@H](C)NC(=O)N1c1nc2c3c(ccc2s1)OCC3] | (4R,5R)-1-(7,8-dihydrobenzofuro[4,5- d]thiazol-2-yl)-4-methyl-5-(prop-1-yn-1- yl)imidazolidin-2-one |
| 17 | 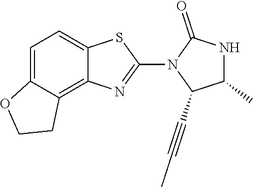 [SMILES: CC#C[C@H]1[C@@H](C)NC(=O)N1c1nc2c3c(ccc2s1)OCC3] | (4R,5S)-1-(7,8-dihydrobenzofuro[4,5- d]thiazol-2-yl)-4-methyl-5-(prop-1-yn-1- yl)imidazolidin-2-one |
| 18 | 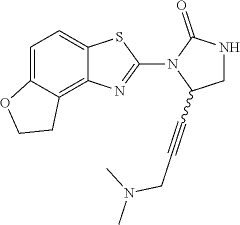 [SMILES: CN(C)CC#CC1CNC(=O)N1c1nc2c3c(ccc2s1)OCC3] | (RS)-1-(7,8-dihydrobenzofuro[4,5- d]thiazol-2-yl)-5-[3- (dimethylamino)prop-1-yn-1- yl]imidazolidin-2-one |
| 19 | 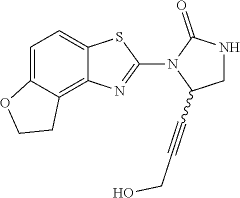 [SMILES: O=C1NCC(C#CCO)N1c1nc2c3c(ccc2s1)OCC3] | (RS)-1-(7,8-dihydrobenzofuro[4,5- d]thiazol-2-yl)-5-(3-hydroxyprop-1-yn-1- yl)imidazolidin-2-one |
| 20 | 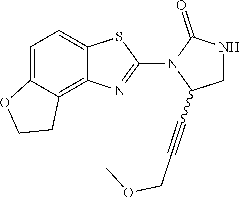 [SMILES: COCC#CC1CNC(=O)N1c1nc2c3c(ccc2s1)OCC3] | (RS)-1-(7,8-dihydrobenzofuro[4,5- d]thiazol-2-yl)-5-(3-methoxyprop-1-yn-1- yl)imidazolidin-2-one |
| 21 | 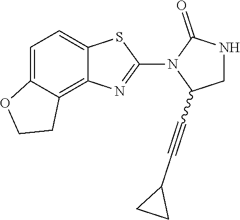 [SMILES: O=C1NCC(C#CC2CC2)N1c1nc2c3c(ccc2s1)OCC3] | (RS)-5-(cyclopropylethynyl)-1-(7,8- dihydrobenzofuro[4,5-d]thiazol-2- yl)imidazolidin-2-one |
| 22 | 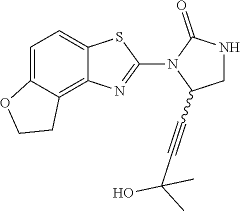 [SMILES: CC(C)(O)C#CC1CNC(=O)N1c1nc2c3c(ccc2s1)OCC3] | (RS)-1-(7,8-dihydrobenzofuro[4,5- d]thiazol-2-yl)-5-(3-hydroxy-3- methylbut-1-yn-1-yl)imidazolidin-2-one |

### Example 23

### Production of (4S,5R)-1-([1,3]dioxolo[4′,5′:5,6]benzo[1,2-d]thiazol-7-yl)-4-methyl-5-(prop-1-yn-1-yl)imidazolidin-2-one

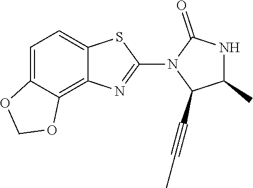 [SMILES: CC#C[C@@H]1[C@H](C)NC(=O)N1c1nc2c3c(ccc2s1)OCO3]

(First Step)

To a solution of tert-butyl ((2S,3RS)-3-aminohex-4-yn-2-yl)carbamate (1.0 g, 4.72 mmol) in DCM (30 mL) obtained in the fourth step of Example 4, TEA (1.32 mL, 9.43 mmol) and N,N′-disuccinimidyl carbonate (1.32 g, 5.17 mmol) were added. After the mixture was stirred for 4 hours at room temperature, water was added to the reaction liquid and extracted with ethyl acetate. The organic layer was washed with water and saturated brine and dried over anhydrous sodium sulfate. After the solvent was removed under reduced pressure, the residue was purified by column chromatography (silica gel, petroleum ether/ethyl acetate) to afford tert-butyl (4RS,5S)-5-methyl oxo-4-(prop-1-yn-1-yl)imidazolidine-1-carboxylate (yield: 0.6 g).

(Second Step)

To a solution of tert-butyl (4RS,5S)-5-methyl-2-oxo-4-(prop-1-yn-1-yl)imidazolidine-1-carboxylate (0.74 g, 3.1 mmol) in acetonitrile (30 mL) obtained by repeating the first step, cesium carbonate (1.09 g, 3.36 mmol) and 7-(ethylsulfonyl)-[1,3]dioxolo[4′,5′:5,6]benzo[1,2-d]thiazole (700 mg, 2.58 mmol) were added. After the mixture was stirred for 8 hours at 80° C., water was added to the reaction liquid and extracted with ethyl acetate. The organic layer was washed with water and saturated brine and dried over anhydrous sodium sulfate. After the solvent was removed under reduced pressure, the residue was purified by column chromatography (silica gel, petroleum ether/ethyl acetate) to afford tert-butyl (4RS,5S)-3-([1,3]dioxolo[4′,5′:5,6]benzo[1,2-d]thiazol-7-yl)-5-methyl-2-oxo-4-(prop-1-yn-1-yl)imidazolidine-1-carboxylate (yield: 0.3 g).

NMR (DMSO-d₆) δ=7.49-7.45 (m, 1H), 7.05-7.01 (m, 1H), 6.16-6.14 (m, 2H), 5.04-5.02 (m, 1H), 4.30-4.25 (m, 1H), 1.83-1.81 (m, 3H), 1.52-1.50 (m, 9H), 1.40 (d, J=6.4 Hz, 3H); LCMS (m/z): 416.15 [M+H]⁺.

(Third Step)

To a solution of tert-butyl (4RS,5S)-3-([1,3]dioxolo[4′,5′:5,6]benzo[1,2-d]thiazol-7-yl)-5-methyl-2-oxo-4-(prop-1-yn-1-yl)imidazolidine-1-carboxylate (0.3 g, 0.72 mmol) in DCM (15 mL), trifluoroacetic acid (1.6 mL, 21.68 mmol) was added under ice cooling and stirred for 4 hours at room temperature. The reaction liquid was concentrated under reduced pressure, and ice water was added to the residue and neutralized with a saturated aqueous solution of sodium hydrogencarbonate. The precipitate was collected by filtration and dried under reduced pressure to afford (4S,5RS)-1-([1,3]dioxolo[4′,5′:5,6]benzo[1,2-d]thiazol-7-yl)-4-methyl-5-(prop-1-yn-1-yl)imidazolidin-2-one as a diastereomeric mixture (yield: 0.19 g). The resulting diastereomeric mixture was purified by using supercritical fluid chromatography (Lux Cellulose-2 (30×250 mm), carbon dioxide/methanol) to afford the titled compound (yield: 41 mg).

¹H NMR (DMSO-d₆) δ=8.05 (s, 1H), 7.37 (d, J=8.0 Hz, 1H), 6.94 (d, J=8.3 Hz, 1H), 6.12 (d, J=2.9 Hz, 2H), 5.33 (d, J=5.9 Hz, 1H), 4.08 (quin, J=6.4 Hz, 1H), 1.82 (s, 3H), 1.29 (d, J=6.1 Hz, 3H); LCMS (m/z): 316.08[M+H]⁺; in supercritical fluid chromatography (Chiralpak IC-3 (4.6×150 mm), 0.5% DEA in Methanol), retention time: 4.77 minutes.

TABLE 2

| Example | | |
|---|---|---|
| Number | Structure | Compound Name |
| 24 | 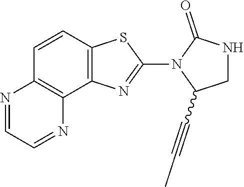 [SMILES: CC#CC1CNC(=O)N1c1nc2c(ccc3nccnc32)s1] | (RS)-5-(prop-1-yn-1-yl)-1-(thiazolo[4,5- f]quinoxalin-2-yl)imidazolidin-2-one |
| 25 | 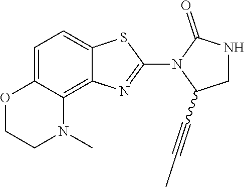 [SMILES: CC#CC1CNC(=O)N1c1nc2c3c(ccc2s1)OCCN3C] | (RS)-1-(9-methyl-8,9-dihydro-7H- thiazolo[4′,5′;3,4]benz[1,2-b][1,4]oxazin- 2-yl)-5-(prop-1-yn-1-yl)imidazolidin-2- one |
| 26 | 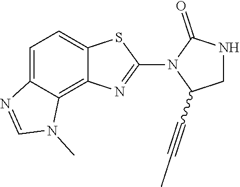 [SMILES: CC#CC1CNC(=O)N1c1nc2c(ccc3ncn(C)c32)s1] | (RS)-1-(8-methyl-8H- imidazo[4′,5′:5,6]benz[1,2-d]thiazol-2- yl)-5-(prop-1-yn-1-yl)imidazolidin-2-one |
| 27 | 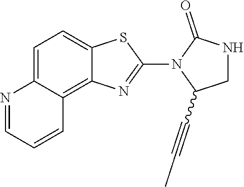 [SMILES: CC#CC1CNC(=O)N1c1nc2c(ccc3ncccc32)s1] | (RS)-5-(prop-1-yn-1-yl)-1-(thiazolo[4,5- f]quinolin-2-yl)imidazolidin-2-one |
| 28 | 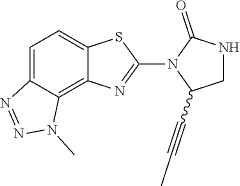 [SMILES: CC#CC1CNC(=O)N1c1nc2c(ccc3nnn(C)c32)s1] | (RS)-1-(1-methyl-1H- thiazolo[4′,5′:3,4]benz[1,2- d][1,2,3]triazol-7-yl)-5-(prop-1-yn-1- yl)imidazolidin-2-one |
| 29 (Enantiomer A of 28) | 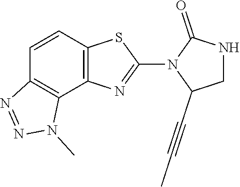 [SMILES: CC#CC1CNC(=O)N1c1nc2c(ccc3nnn(C)c32)s1] | 1-(1-methyl-1H- thiazolo[4′,5′:3,4]benz[1,2- d][1,2,3]triazol-7-yl)-5-(prop-1-yn-1- yl)imidazolidin-2-one |
| 30 (Enantiomer B of 28) | 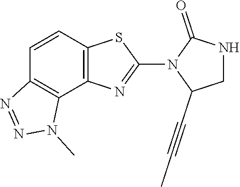 [SMILES: CC#CC1CNC(=O)N1c1nc2c(ccc3nnn(C)c32)s1] | 1-(1-methyl-1H- thiazolo[4′,5′:3,4]benz[1,2-d][1,2,3]triazol- 7-yl)-5-(prop-1-yn-1-yl)imidazolidin-2-one |
| 31 | 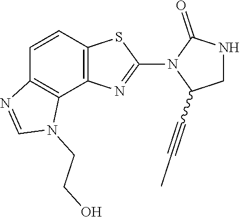 [SMILES: CC#CC1CNC(=O)N1c1nc2c(ccc3ncn(CCO)c32)s1] | (RS)-1-[8-(2-hydroxyethyl)-8H- imidazo[4′,5′:5,6]benz[1,2-d]thiazol-2-yl]- 5-(prop-1-yn-1-yl)imidazolidin-2-one |
| 32 | 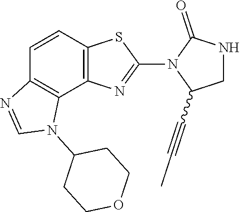 [SMILES: CC#CC1CNC(=O)N1c1nc2c(ccc3ncn(C4CCOCC4)c32)s1] | (RS)-5-(prop-1-yn-1-yl)-1-[8-(tetrahydro- 2H-pyran-4-yl)-8H-imidazo[4′,5′:5,6]benz [1,2-d]thiazol-2-yl]imidazolidin-2-one |
| 33 | 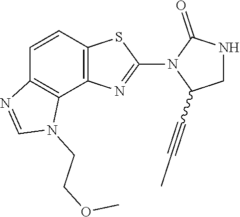 [SMILES: CC#CC1CNC(=O)N1c1nc2c(ccc3ncn(CCOC)c32)s1] | (RS)-1-[8-(2-methoxyethyl)-8H- imidazo[4′,5′:5,6]benz[1,2-d]thiazol-2-yl]- 5-(prop-1-yn-1-yl)imidazolidin-2-one |
| 34 (Enantiomer A of 27) | 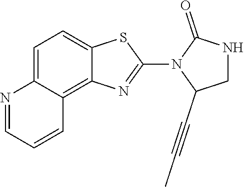 [SMILES: CC#CC1CNC(=O)N1c1nc2c(ccc3ncccc32)s1] | 5-(prop-1-yn-1-yl)-1-(thiazolo[4,5- f]quinolin-2-yl)imidazolidin-2-one |
| 35 | 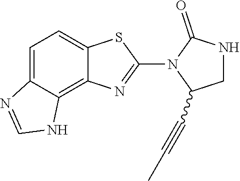 [SMILES: CC#CC1CNC(=O)N1c1nc2c(ccc3nc[nH]c32)s1] | (RS)-1-(8H-imidazo[4′,5′:5,6]benz[1,2- d]thiazol-2-yl)-5-(prop-1-yn-1- yl)imidazolidin-2-one |
| 36 (Enantiomer B of 27) | 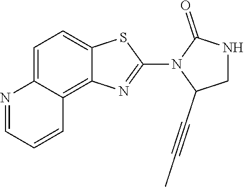 [SMILES: CC#CC1CNC(=O)N1c1nc2c(ccc3ncccc32)s1] | 5-(prop-1-yn-1-yl)-1-(thiazolo[4,5- f]quinolin-2-yl)imidazolidin-2-one |
| 37 | 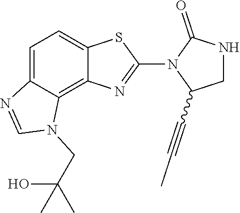 [SMILES: CC#CC1CNC(=O)N1c1nc2c(ccc3ncn(CC(C)(C)O)c32)s1] | (RS)-1-[8-(2-hydroxy-2-methyl propyl)- 8H-imidazo[4′,5′;5,6]benz[1,2-d]thiazol-2- yl]-5-(prop-1-yn-1-yl)imidazolidin-2-one |
| 38 | 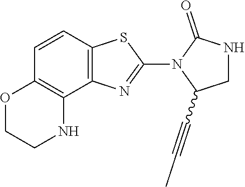 [SMILES: CC#CC1CNC(=O)N1c1nc2c3c(ccc2s1)OCCN3] | (RS)-1-(8,9-dihydro-7H- thiazolo[4′,5′:3,4]benz[1,2-b][1,4]oxazin- 2-yl)-5-(prop-1-yn-1-yl)imidazolidin-2-one |
| 39 | 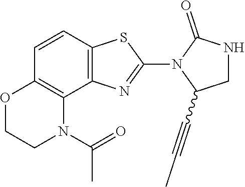 [SMILES: CC#CC1CNC(=O)N1c1nc2c3c(ccc2s1)OCCN3C(C)=O] | (RS)-1-(9-acetyl-8,9-dihydro-7H- thiazolo[4′,5′:3,4]benz[1,2-b][1,4]oxazin- 2-yl)-5-(prop-1-yn-1-yl)imidazolidin-2-one |
| 40 | 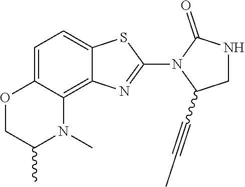 [SMILES: CC#CC1CNC(=O)N1c1nc2c3c(ccc2s1)OCC(C)N3C] | (RS)-1-((RS)-8,9-dimethyl-8,9-dihydro- 7H-thiazolo[4′,5′:3,4]benz[1,2- b][1,4]oxazin-2-yl)-5-(prop-1-yn-1- yl)imidazolidin-2-one |
| 41 | 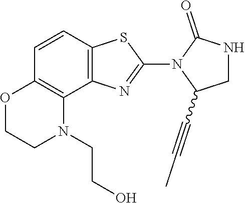 [SMILES: CC#CC1CNC(=O)N1c1nc2c3c(ccc2s1)OCCN3CCO] | (RS)-1-[9-(2-hydroxyethyl)-8,9-dihydro- 7H-thiazolo[4′,5′:3,4]benz[1,2- b][1,4]oxazin-2-yl]-5-(prop-1-yn-1- yl)imidazolidin-2-one |
| 42 | 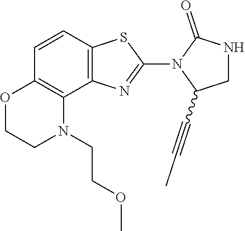 [SMILES: CC#CC1CNC(=O)N1c1nc2c3c(ccc2s1)OCCN3CCOC] | (RS)-1-[9-(2-methoxyethyl)-8,9-dihydro- 7H-thiazolo[4′,5′:3,4]benz[1,2- b][1,4]oxazin-2-yl]-5-(prop-1-yn-1- yl)imidazolidin-2-one |
| 43 | 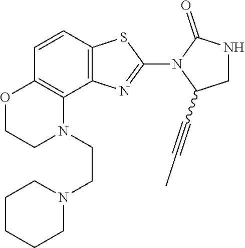 [SMILES: CC#CC1CNC(=O)N1c1nc2c3c(ccc2s1)OCCN3CCN1CCCCC1] | (RS)-1-{9-[2-(piperidin-1-yl)ethyl]-8,9- dihydro-7H-thiazolo[4′,5′:3,4]benz[1,2- b][1,4]oxazin-2-yl}-5-(prop-1-yn-1- yl)imidazolidin-2-one |
| 44 | 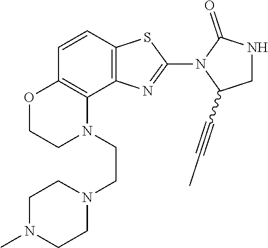 [SMILES: CC#CC1CNC(=O)N1c1nc2c3c(ccc2s1)OCCN3CCN1CCN(C)CC1] | (RS)-1-{9-[2-(4-methyl piperazin-1- yl)ethyl]-8,9-dihydro-7H- thiazolo[4′,5′:3,4]benz[1,2-b][1,4]oxazin- 2-yl}-5-(prop-1-yn-1-yl)imidazolidin-2- one |
| 45 | 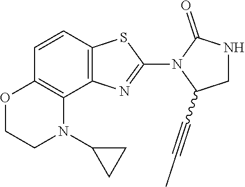 [SMILES: CC#CC1CNC(=O)N1c1nc2c3c(ccc2s1)OCCN3C1CC1] | (RS)-1-(9-cyclopropyl-8,9-dihydro-7H- thiazolo[4′,5′:3,4]benz[1,2-b][1,4]oxazin- 2-yl)-5-(prop-1-yn-1-yl)imidazolidin-2-one |
| 46 | 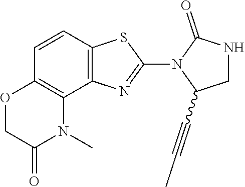 [SMILES: CC#CC1CNC(=O)N1c1nc2c3c(ccc2s1)OCC(=O)N3C] | (RS)-9-methyl-2-[2-oxo-5-(prop-1-yn-1- yl)imidazolidin-1-yl]-7H- thiazolo[4′,5′:3,4]benz[1,2-b][1,4]oxazin- 8(9H)-one |
| 47 | 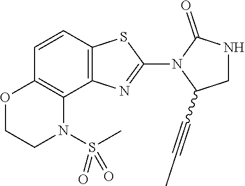 [SMILES: CC#CC1CNC(=O)N1c1nc2c3c(ccc2s1)OCCN3S(C)(=O)=O] | (RS)-1-[9-(methylsulfonyl)-8,9-dihydro- 7H-thiazolo[4′,5′:3,4]benz[1,2- b][1,4]oxazin-2-yl]-5-(prop-1-yn-1- yl)imidazolidin-2-one |
| 48 (Diastereomer A of 40) | 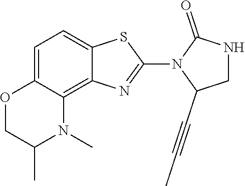 [SMILES: CC#CC1CNC(=O)N1c1nc2c3c(ccc2s1)OCC(C)N3C] | 1-(-8,9-dimethyl-8,9-dihydro-7H- thiazolo[4′,5′:3,4]benz[1,2-b][1,4]oxazin- 2-yl)-5-(prop-1-yn-1-yl)imidazolidin-2- one |
| 49 (Diastereomer B of 40) | 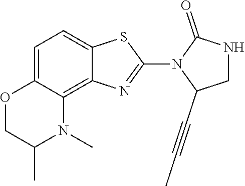 [SMILES: CC#CC1CNC(=O)N1c1nc2c3c(ccc2s1)OCC(C)N3C] | 1-(-8,9-dimethyl-8,9-dihydro-7H- thiazolo[4′,5′:3,4]benz[1,2-b][1,4]oxazin- 2-yl)-5-(prop-1-yn-1-yl)imidazolidin-2- one |
| 50 | 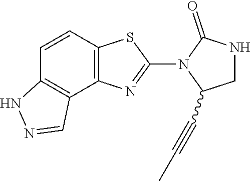 [SMILES: CC#CC1CNC(=O)N1c1nc2c(ccc3[nH]ncc32)s1] | (RS)-5-(prop-1-yn-1-yl)-1-(6H- thiazolo[4,5-e]indazol-2-yl)imidazolidin- 2-one |
| 51 | 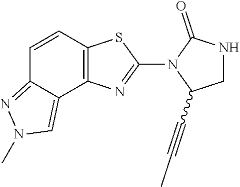 [SMILES: CC#CC1CNC(=O)N1c1nc2c(ccc3nn(C)cc32)s1] | (RS)-1-(7-methyl-7H-thiazolo[4,5- e]indazol-2-yl)-5-(prop-1-yn-1- yl)imidazolidin-2-one |
| 52 (Enantiomer A of 7) | 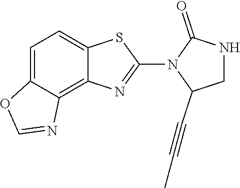 [SMILES: CC#CC1CNC(=O)N1c1nc2c(ccc3ocnc32)s1] | 5-(prop-1-yn-1-yl)-1- (thiazolo[5′,4′:5,6]benz[1,2-d]oxazol-7- yl)imidazolidin-2-one |
| 53 (Enantiomer B of 7) | 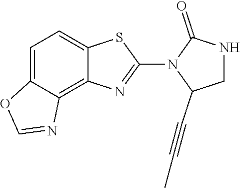 [SMILES: CC#CC1CNC(=O)N1c1nc2c(ccc3ocnc32)s1] | 5-(prop-1-yn-1-yl)-1- (thiazolo[5′,4′:5,6]benz[1,2-d]oxazol-7- yl)imidazolidin-2-one |
| 54 | 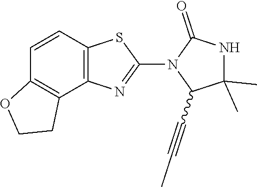 [SMILES: CC#CC1N(c2nc3c4c(ccc3s2)OCC4)C(=O)NC1(C)C] | (RS)-1-(7,8-dihydrobenzofuro[4,5- d]thiazol-2-yl)-4,4-dimethyl-5-(prop-1- yn-1-yl)imidazolidin-2-one |

### Example 55

### Production of (RS)-1-(7,8-dihydro-[1,4]dioxyno[2′,3′:5,6]benzo[1,2-d]thiazol-2-yl)-5-(prop-1-yn-1-yl)imidazolidin-2-one

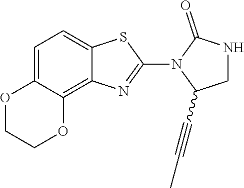 [SMILES: CC#CC1CNC(=O)N1c1nc2c3c(ccc2s1)OCCO3]

(First Step)

To a solution of 2-(ethylsulfonyl)-7,8-dihydro-[1,4]dioxyno[2′,3′:5,6]benzo[1,2-d]thiazole (55 mg, 0.19 mmol) and tert-butyl (RS)-2-oxo-4-(prop-1-yn-1-yl)imidazolidine-1-carboxylate (50 mg, 0.22 mmol) in toluene (4 mL), tripotassium phosphate (62 mg, 0.29 mmol) was added at room temperature and stirred for 3 hours at 50° C., for 2 hours at 70° C., and for 10 hours at 80° C. Water was added to the reaction solution, the mixture was extracted with chloroform, then the organic layer was washed with saturated brine and dried over anhydrous magnesium sulfate. After the solvent was removed under reduced pressure, the residue was purified by column chromatography (silica gel, chloroform/ethyl acetate) to afford tert-butyl (RS)-3-(7,8-dihydro-[1,4]dioxyno[2′,3′:5,6]benzo[1,2-d]thiazol-2-yl)-2-oxo-4-(prop-1-yn-1-yl)imidazolidine carboxylate (yield: 0.066 g).

¹H-NMR (CDCl₃) δ: 7.21 (d, J=8.5 Hz, 1H), 6.88 (d, J=8.5 Hz, 1H), 5.45-5.39 (m, 1H), 4.47-4.42 (m, 2H), 4.34-4.29 (m, 2H), 4.08-3.97 (m, 2H), 1.77 (d, J=2.4 Hz, 3H), 1.57 (s, 9H).

(Second Step)

To tert-butyl (RS)-3-(7,8-dihydro-[1,4]dioxyno[2′,3′:5,6]benzo[1,2-d]thiazol-2-yl)-2-oxo-4-(prop-1-yn-1-yl)imidazolidine-1-carboxylate (66 mg, 0.16 mmol), TFA (4 mL) was added at room temperature and stirred for 15 minutes. The reaction solution was removed under reduced pressure, saturated sodium bicarbonate solution was added thereto, and the mixture was extracted with chloroform/ethanol (3/1), the organic layer was dried over anhydrous magnesium sulfate. After the solvent was removed under reduced pressure, chloroform was added to the residue, and the precipitate was collected by filtration, washed with chloroform, and then dried to afford the titled compound (yield: 26 mg).

¹H-NMR (DMSO-d₆) δ: 7.96 (br s, 1H), 7.29 (d, J=8.5 Hz, 1H), 6.81 (d, J=8.5 Hz, 1H), 5.32-5.28 (m, 1H), 4.41-4.27 (m, 4H), 3.84-3.77 (m, 1H), 3.43-3.39 (m, 1H), 1.78 (d, J=2.4 Hz, 3H). LCMS (m/z): 316.1 [M+H]⁺.

TABLE 3

| | | |
|---|---|---|
| 56 | 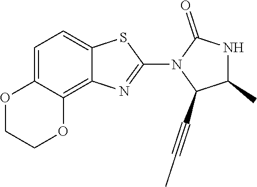 [SMILES: CC#C[C@@H]1[C@H](C)NC(=O)N1c1nc2c3c(ccc2s1)OCCO3] | (4S,5R)-1-(7,8-dihydro- [1,4]dioxino[2′,3′:5,6]benz[1,2- d]thiazol-2-yl)-4-methyl-5-(prop-1-yn- 1-yl)imidazolidin-2-one |
| 57 | 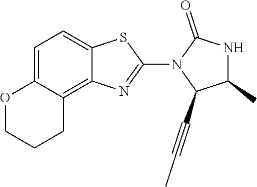 [SMILES: CC#C[C@@H]1[C@H](C)NC(=O)N1c1nc2c3c(ccc2s1)OCCC3] | (4S,5R)-1-(8,9-dihydro-7H- chromeno[5,6-d]thiazol-2-yl)-4- methyl-5-(prop-1-yn-1- yl)imidazolidin-2-one |
| 58 (Enantiomer A of 55) | 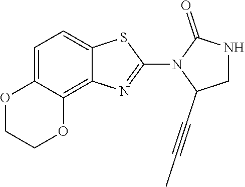 [SMILES: CC#CC1CNC(=O)N1c1nc2c3c(ccc2s1)OCCO3] | 1-(7,8-dihydro- [1,4]dioxino[2′,3′:5,6]benz[1,2- d]thiazol-2-yl)-5-(prop-1-yn-1- yl)imidazolidin-2-one |
| 59 (Enantiomer B of 55) | 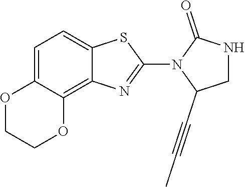 [SMILES: CC#CC1CNC(=O)N1c1nc2c3c(ccc2s1)OCCO3] | 1-(7,8-dihydro- [1,4]dioxino[2′,3′:5,6]benz[1,2- d]thiazol-2-yl)-5-(prop-1-yn-1- yl)imidazolidin-2-one |
| 60 (Diastereomer of 61) | 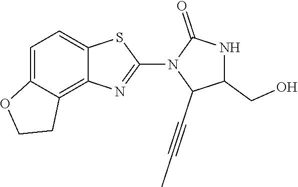 [SMILES: CC#CC1C(CO)NC(=O)N1c1nc2c3c(ccc2s1)OCC3] | cis-1-(7,8-dihydrobenzofuro[4,5- d]thiazol-2-yl)-4-(hydroxymethyl)-5- (prop-1-yn-1-yl)imidazolidin-2-one |
| 61 (Diastereomer of 60) | 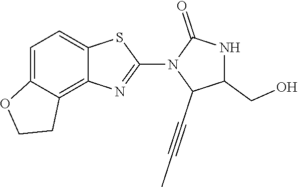 [SMILES: CC#CC1C(CO)NC(=O)N1c1nc2c3c(ccc2s1)OCC3] | trans-1-(7,8-dihydrobenzofuro[4,5- d]thiazol-2-yl)-4-(hydroxymethyl)-5- (prop-1-yn-1-yl)imidazolidin-2-one |
| 62 | 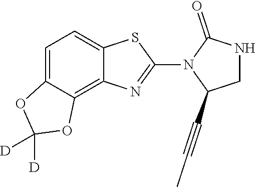 [SMILES: [2H]C1([2H])Oc2ccc3sc(N4C(=O)NC[C@H]4C#CC)nc3c2O1] | (R)-1- ([1,3]dioxolo[4′,5′:5,6]benz[1,2- d]thiazol-7-yl-2,2-d2)-5-(prop-1- yn-1-yl)imidazolidin-2-one |
| 63 | 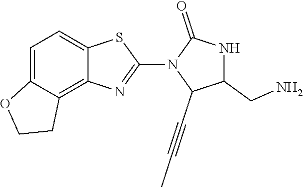 [SMILES: CC#CC1C(CN)NC(=O)N1c1nc2c3c(ccc2s1)OCC3] | 4-(aminomethyl)-1-(7,8- dihydrobenzofuro[4,5-d]thiazol-2- yl)-5-(prop-1-yn-1-yl)imidazolidin- 2-one |
| 64 | 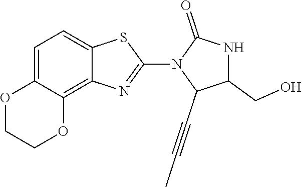 [SMILES: CC#CC1C(CO)NC(=O)N1c1nc2c3c(ccc2s1)OCCO3] | cis-1-(7,8-dihydro- [1,4]dioxino[2′,3′:5,6]benz[1,2- d]thiazol-2-yl)-4-(hydroxymethyl)- 5-(prop-1-yn-1-yl)imidazolidin-2- one |
| 65 (Enantiomer of 66) | 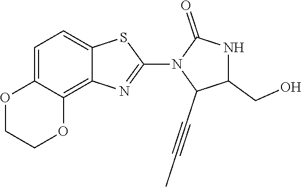 [SMILES: CC#CC1C(CO)NC(=O)N1c1nc2c3c(ccc2s1)OCCO3] | trans-1-(7,8-dihydro- [1,4]dioxino[2′,3′:5,6]benz[1,2- d]thiazol-2-yl)-4-(hydroxymethyl)- 5-(prop-1-yn-1-yl)imidazolidin-2- one |
| 66 (Enantiomer of 65) | 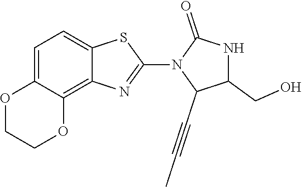 [SMILES: CC#CC1C(CO)NC(=O)N1c1nc2c3c(ccc2s1)OCCO3] | trans-1-(7,8-dihydro- [1,4]dioxino[2′,3′:5,6]benz[1,2- d]thiazol-2-yl)-4-(hydroxymethyl)- 5-(prop-1-yn-1-yl)imidazolidin-2- one |
| 67 | 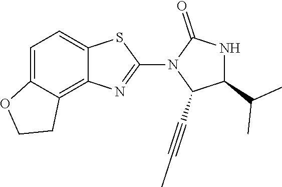 [SMILES: CC#C[C@H]1[C@H](C(C)C)NC(=O)N1c1nc2c3c(ccc2s1)OCC3] | (4S,5S)-1-(7,8- dihydrobenzofuro[4,5-d]thiazol-2- yl)-4-isopropyl-5-(prop-1-yn-1- yl)imidazolidin-2-one |
| 68 | 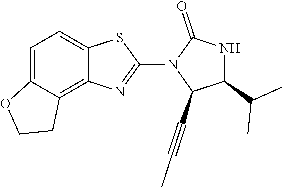 [SMILES: CC#C[C@@H]1[C@H](C(C)C)NC(=O)N1c1nc2c3c(ccc2s1)OCC3] | (4S,5R)-1-(7,8- dihydrobenzofuro[4,5-d]thiazol-2- yl)-4-isopropyl-5-(prop-1-yn-1- yl)imidazolidin-2-one |
| 69 (Enantiomer of 70) | 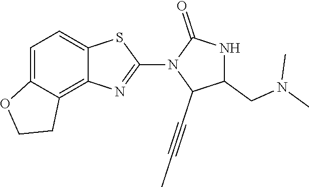 [SMILES: CC#CC1C(CN(C)C)NC(=O)N1c1nc2c3c(ccc2s1)OCC3] | trans-1-(7,8-dihydrobenzofuro[4,5- d]thiazol-2-yl)-4- [(dimethylamino)methyl]-5-(prop- 1-yn-1-yl)imidazolidin-2-one |
| 70 (Enantiomer of 69) | 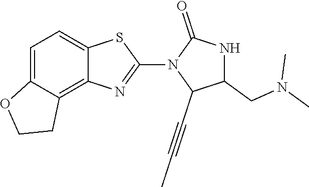 [SMILES: CC#CC1C(CN(C)C)NC(=O)N1c1nc2c3c(ccc2s1)OCC3] | trans-1-(7,8-dihydrobenzofuro[4,5- d]thiazol-2-yl)-4- [(dimethylamino)methyl]-5-(prop- 1-yn-1-yl)imidazolidin-2-one |
| 71 (Enantiomer of 72) | 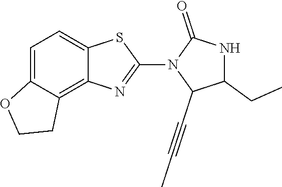 [SMILES: CC#CC1C(CC)NC(=O)N1c1nc2c3c(ccc2s1)OCC3] | trans-1-(7,8-dihydrobenzofuro[4,5- d]thiazol-2-yl)-4-ethyl-5-(prop-1- yn-1-yl)imidazolidin-2-one |
| 72 (Enantiomer of 71) | 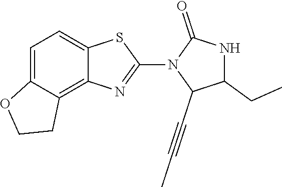 [SMILES: CC#CC1C(CC)NC(=O)N1c1nc2c3c(ccc2s1)OCC3] | trans-1-(7,8-dihydrobenzofuro[4,5- d]thiazol-2-yl)-4-ethyl-5-(prop-1- yn-1-yl)imidazolidin-2-one |
| 73 | 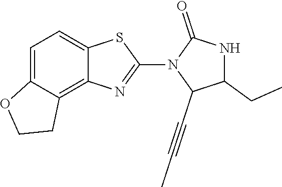 [SMILES: CC#CC1C(CC)NC(=O)N1c1nc2c3c(ccc2s1)OCC3] | cis-1-(7,8-dihydrobenzofuro[4,5- d]thiazol-2-yl)-4-ethyl-5-(prop-1- yn-1-yl)imidazolidin-2-one |
| 74 (Enantiomer of 75) | 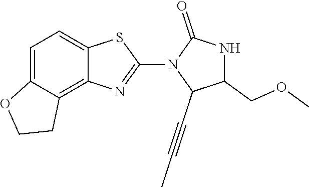 [SMILES: CC#CC1C(COC)NC(=O)N1c1nc2c3c(ccc2s1)OCC3] | cis-1-(7,8-dihydrobenzofuro[4,5- d]thiazol-2-yl)-4-(methoxymethyl)- 5-(prop-1-yn-1-yl)imidazolidin-2- one |
| 75 (Enantiomer of 74) | 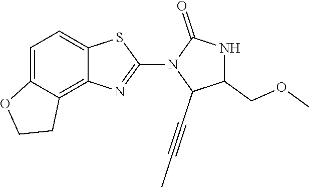 [SMILES: CC#CC1C(COC)NC(=O)N1c1nc2c3c(ccc2s1)OCC3] | cis-1-(7,8-dihydrobenzofuro[4,5- d]thiazol-2-yl)-4-(methoxymethyl)- 5-(prop-1-yn-1-yl)imidazolidin-2- one |
| 76 (Enantiomer of 77) | 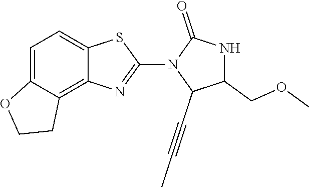 [SMILES: CC#CC1C(COC)NC(=O)N1c1nc2c3c(ccc2s1)OCC3] | trans-1-(7,8-dihydrobenzofuro[4,5- d]thiazol-2-yl)-4-(methoxymethyl)- 5-(prop-1-yn-1-yl)imidazolidin-2- one |
| 77 (Enantiomer of 76) | 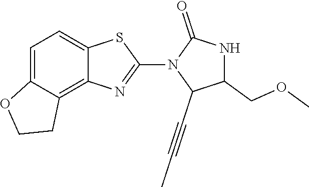 [SMILES: CC#CC1C(COC)NC(=O)N1c1nc2c3c(ccc2s1)OCC3] | trans-1-(7,8-dihydrobenzofuro[4,5- d]thiazol-2-yl)-4-(methoxymethyl)- 5-(prop-1-yn-1-yl)imidazolidin-2- one |
| 78 (Enantiomer of 79) | 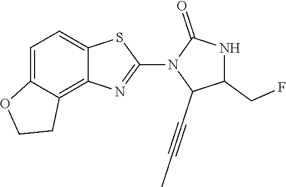 [SMILES: CC#CC1C(CF)NC(=O)N1c1nc2c3c(ccc2s1)OCC3] | trans-1-(7,8-dihydrobenzofuro[4,5- d]thiazol-2-yl)-4-(fluoromethyl)- 5-(prop-1-yn-1-yl)imidazolidin-2- one |
| 79 (Enantiomer of 78) | 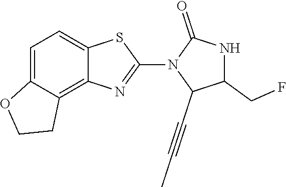 [SMILES: CC#CC1C(CF)NC(=O)N1c1nc2c3c(ccc2s1)OCC3] | trans-1-(7,8-dihydrobenzofuro[4,5- d]thiazol-2-yl)-4-(fluoromethyl)-5- (prop-1-yn-1-yl)imidazolidin-2-one |
| 80 | 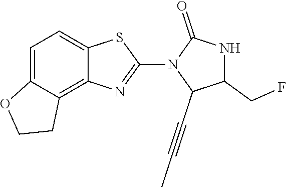 [SMILES: CC#CC1C(CF)NC(=O)N1c1nc2c3c(ccc2s1)OCC3] | cis-1-(7,8-dihydrobenzofuro[4,5- d]thiazol-2-yl)-4-(fluoromethyl)-5- (prop-1-yn-1-yl)imidazolidin-2-one |
| 81 | 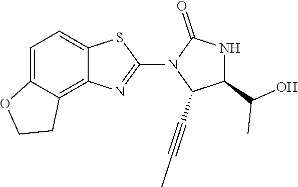 [SMILES: CC#C[C@H]1[C@H](C(C)O)NC(=O)N1c1nc2c3c(ccc2s1)OCC3] | (4R,5S)-1-(7,8- dihydrobenzofuro[4,5-d]thiazol-2- yl)-4-((R)-1-hydroxyethyl)-5-(prop- 1-yn-1-yl)imidazolidin-2-one |
| 82 | 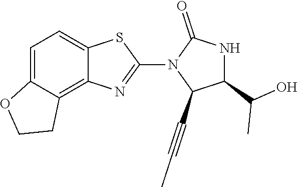 [SMILES: CC#C[C@@H]1[C@H](C(C)O)NC(=O)N1c1nc2c3c(ccc2s1)OCC3] | (4R,5R)-1-(7,8- dihydrobenzofuro[4,5-d]thiazol-2- yl)-4-((R)-1-hydroxyethyl)-5-(prop- 1-yn-1-yl)imidazolidin-2-one |
| 83 | 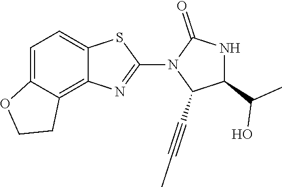 [SMILES: CC#C[C@H]1[C@H](C(C)O)NC(=O)N1c1nc2c3c(ccc2s1)OCC3] | (4R,5S)-1-(7,8- dihydrobenzofuro[4,5-d]thiazol-2- yl)-4-((S)-1-hydroxyethyl)-5-(prop- 1-yn-1-yl)imidazolidin-2-one |
| 84 | 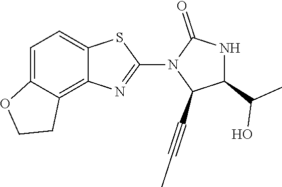 [SMILES: CC#C[C@@H]1[C@H](C(C)O)NC(=O)N1c1nc2c3c(ccc2s1)OCC3] | (4R,5R)-1-(7,8- dihydrobenzofuro[4,5-d]thiazol-2- yl)-4-((S)-1-hydroxyethyl)-5-(prop- 1-yn-1-yl)imidazolidin-2-one |

TABLE 4

| Example | | LCMS m/z |
|---|---|---|
| No. | ¹H NMR δ (ppm) | [M + H]⁻ |
| 8 | ¹H NMR (DMSO-d₆) δ (ppm) 8.00 (br. s, 1H), 7.61 | 300.3 |
| | (d, J = 8.4 Hz, 1H), 6.76 (d, J = 8.4 Hz, 1H), | |
| | 5.31 − 5.38 (m, 1H), 4.63 (t, J = 8.9 Hz, 2H), | |
| | 3.82 (t, J = 9.1 Hz, 1H), 3.28 − 3.46 (m, 3H), | |
| | 1.79 (d, J = 2.1 Hz, 3H) | |
| 9 | ¹H NMR (DMSO-d₆) δ = 7.98 (s, 1H), 7.61 (d, J = | 300.3 |
| | 8.3 Hz, 1H), 6.76 (d, J = 8.5 Hz, 1H), 5.34 (qd, | |
| | J = 2.2, 6.5 Hz, 1H), 4.63 (t, J = 8.8 Hz, 2H), | |
| | 3.82 (t, J = 9.0 Hz, 1 H), 3.44 − 3.35 (m, 3H), | |
| | 1.79 (d, J = 2.0 Hz, 3H). | |
| 10 | ¹H NMR (DMSO-d₆) δ = 7.99 (s, 1H), 7.61 (d, J = | 300.3 |
| | 8.5 Hz, 1H), 6.76 (d, J = 8.3 Hz, 1H), 5.37 − | |
| | 5.31 (m, 1H), 4.63 (t, J = 8. 8 Hz, 2H), 3.82 (t, | |
| | J = 9.0 Hz, 1H), 3.44 − 3.35 (m, 3H), 1.79 (d, | |
| | J = 2.2 Hz, 3H). | |
| 11 | ¹H NMR (DMSO-d₆) δ = 8.04 (s, 1H), 7.62 (d, J = | 358.1 |
| | 8.42 Hz, 1H), 6.77 (d, J = 8.40 Hz, 1H), 5.42 | |
| | (dd, J = 3.31, 9.16 Hz, 1H), 4.70 − 4.56 (m, 2H), | |
| | 3.90 − 3.80 (m, 1H), 3.51 − 3.26 (m, 3H), 0.11 (s, 911). | |
| 12 | 1H NMR (DMSO-d6) δ 8.06 (s, 1H), 7.66 − 7.58 (m, | 286.1 |
| | 1H), 6.77 (d, J = 8.38 Hz, 1H), 5.39 (dt, J = | |
| | 2.38, 8.93 Hz, 1H), 4.67 − 4.58 (m, 2H), 3.84 | |
| | (t, J = 9.01 Hz, 1H), 3.52 − 3.44 (m, 2H), 3.42 − 3.35 (m, 2H). | |
| 13 | ¹H NMR (DMSO-d₆) δ = 8.05 (s, 1H), 7.62 (d, J = | 286.2 |
| | 8.3 Hz, 1H), 6.77 (d, J = 8.6 Hz, 1H), 5.39 (td, | |
| | J = 2.2, 8.8 Hz, 1H), 4.62 (t, J = 8.9 Hz, 2H), | |
| | 3.85 (t, J = 9.2 Hz, 1H), 3.50 − 3.44 (m, 2H), | |
| | 3.41 − 3.35 (m, 2H). | |
| 14 | ¹H NMR (DMSO-d₆) δ = 8.05 (s, 1H), 7.62 (d, J = | 286.2 |
| | 8.6 Hz, 1H), 6.77 (d, J = 8.3 Hz, 1H), 5.39 (td, | |
| | J = 2.3, 8.9 Hz, 1H), 4.62 (t, J = 9.0 Hz, 2H), | |
| | 3.85 (t, J = 9. 2 Hz, 1H), 3.50 − 3.44 (m, 2H), | |
| | 3. 38 (t, J = 8. 7 Hz, 2H). | |
| 15 | ¹H NMR (Chloroform-d) δ = 7.50 (d, J = 8.4 Hz, | 362.2 |
| | 1H), 7.42 − 7.36 (m, 2H), 7.32 − 7.27 (m, 3H), | |
| | 6.80 (d, J = 8.4 Hz, 1H), 5.70 − 5.64 (m, 1H), | |
| | 5.03 (s, 1H), 4.72 − 4.65 (m, 2H), 4.06 − 3.99 | |
| | (m, 1H), 3.84 − 3.79 (m, 1H), 3.55 − 3.48 (m, 2H). | |
| 16 | ¹H NMR (500 MHz, DMSO-d₆) δ = 8.18 (br. s, 1H), | 314.1 |
| | 7.61 (d, J = 8.2 Hz, 1H), 6.76 (d, J = 8.5 Hz, | |
| | 1H), 4.91 − 4.86 (m, 1H), 4.62 (t, J = 9.3 Hz, | |
| | 2H), 3.81 − 3.79 (m, 1H), 3.38 (t, J = 8.9 Hz, | |
| | 2H), 1.80 (d, J = 2.1 Hz, 3H), 1.24 (d, J = 6.4 Hz, 3H). | |
| 17 | ¹H NMR (500 MHz, DMSO-d₆) δ = 8.00 (br. s, 1H), | 314.1 |
| | 7.60 (d, J = 8.5 Hz, 1H), 6.76 (d, J = 8.2 Hz, | |
| | 1H), 5.35 (qd, J = 2.0, 7.6 Hz, 1H), 4.62 (t, | |
| | J = 9.0 Hz, 2H), 4.10 − 4.02 (m, 1H), 3.38 (t, | |
| | J = 8.9 Hz, 2H), 1.81 (d, J = 2.4 Hz, 3H), 1.29 | |
| | (d, J = 6.4 Hz, 3H). | |
| 18 | ¹H NMR (500 MHz, DMSO-d₆) δ = 8.02 (m, 1H), 7.61 | 343.2 |
| | (d, J = 8.5 Hz, 1H), 6.76 (d, J = 8.5 Hz, 1H), | |
| | 5.46 − 5.40 (m, 1H), 4.62 (t, J = 9.2 Hz, 2H), | |
| | 3.86 (t, J = 9.2 Hz, 1H), 3.49 − 3.34 (m, 3H), | |
| | 3.22 (s, 2H), 2.10 (s, 6H). | |
| 19 | ¹H NMR (DMSO-d₆) δ = 8.04 (s, 1H), 7.61 (d, J = | 316.1 |
| | 8.4 Hz, 1H), 6.76 (d, J = 8.4 Hz, 1H), 5.48 − | |
| | 5.39 (m, 1H), 5.20 (t, J = 6.0 Hz, 1H), 4.63 (t, | |
| | J = 8.8 Hz, 2H), 4.11 − 4.03 (m, 2H), 3.89 − | |
| | 3.82 (m, 1H), 3.48 − 3.42 (m, 1H), 3.38 (t, J = 8.8 Hz, 2H). | |
| 20 | ¹H NMR (DMSO-d₆) δ = 7.63 (d, J = 8.4 Hz, 1H), | 330.1 |
| | 6.77 (d, J = 8.4 Hz, 1H), 5.42 − 5.36 (m, 1H), | |
| | 5.21 (t, J = 6.0 Hz, 1H), 4.63 (t, J = 8.8 Hz, | |
| | 2H), 4.09 − 4.04 (m, 2H), 3.87 (t, J = 9.0 Hz, | |
| | 1H), 3.58 − 3.51 (m, 1H), 3.38 (t, J = 8.8 Hz, | |
| | 2H), 2.87 (s, 3H). | |
| 21 | ¹H NMR (500 MHz, DMSO-d₆) δ = 8.53 (s, 2H), | 326.1 |
| | 7.99 (s, 1H), 7.61 (d, J = 8.5 Hz, 1H), 6.76 (d, J = | |
| | 8.2 Hz, 1H), 5.32 (d, J = 8.0 Hz, 1H), 4.63 | |
| | (t, J = 9.2 Hz, 2H), 3.80 (t, J = 9.0 Hz, 1H), | |
| | 3.42 − 3.37 (m, 1H), 1.34 − 1.27 (m, 1H), 0.78 − | |
| | 0.73 (m, 2H), 0.61 − 0.51 (m, 2H). | |
| 22 | ¹H NMR (500 MHz, DMSO-d₆) δ = 8.01 (br. s, 1H), | 344.1 |
| | 7.61 (d, J = 8.5 Hz, 1H), 6.76 (d, J = 8.5 Hz, | |
| | 1H), 5.44 − 5.38 (m, 1H), 5.31 (s, 1H), 4.63 (t, | |
| | J = 9.2 Hz, 2H), 3.85 (t, J = 9.0 Hz, 1H), 3.45 − 3.36 | |
| | (m, 3H), 1.33 (s, 3H), 1.30 (s, 3H). | |

TABLE 5

| Example | | LCMS m/z |
|---|---|---|
| No. | ¹H NMR δ (ppm) | [M + H]⁺ |
| 24 | ¹H NMR (DMSO-d₆) δ 9.03 (d, J = 1.9 Hz, 1H), 9.00 | 310.05 |
| | (d, J = 1.9 Hz, 1H), 8.41 (d, J = 8.8 Hz, 1H), | |
| | 8.12 (br. s, 1H), 7.94 (d, J = 8.8 Hz, 1H), | |
| | 5.45 − 5.53 (m, 1H), 3.90 (t, J = 8.9 Hz, 1H), | |
| | 3.46 − 3.53 (m, 1H), 1.78 (d, J = 2.1 Hz, 3H) | |
| 25 | ¹H NMR (DMSO-d₆) δ 7.91 (br. s, 1H), 7.19 (d, | 329.3 |
| | J = 8.5 Hz, 1H), 6.72 (d, J = 8.5 Hz, 1H), 5.24 − | |
| | 5.35 (m, 1H), 4.06 − 4.21 (m, 2H), 3.79 − 3.89 | |
| | (m, 1H), 3.38 − 3.46 (m, 1H), 3.28 (s, 3H), | |
| | 3.20 − 3.25 (m, 2H), 1.77 (d, J = 2.1 Hz, 3H) | |
| 26 | ¹H NMR (DMSO-d₆) δ 8.66 (s, 1H), 8.07 (s, 1H), | 312.1 |
| | 7.84 (d, J = 8.6 Hz, 1H), 7.61 (d, J = 8.7 Hz, | |
| | 1H), 5.51 − 5.29 (m, 1H), 4.29 (s, 3H), 3.96 − | |
| | 3.81 (m, 1H), 3.53 − 3.41 (m, 1H), 1.81 (d, J = | |
| | 2.1 Hz, 3H). | |
| 27 | ¹H NMR (DMSO-d₆) δ = 8.95 − 8.94 (m, 1H), 8.87 | 309.25 |
| | (d, J = 8.3 Hz, 1H), 8.28 (d, J = 9.0 Hz, 1H), | |
| | 8.06 (br, 1H), 7.88 (d, J = 9.0 Hz, 1H), 7.67 | |
| | (dd, J = 4.2, 8.3 Hz, 1H), 5.48 (d, J = 8.8 | |
| | Hz, 1H), 3.90 (t, J = 9.0 Hz, 1H), 3.49 (dd, J = | |
| | 2.6, 9.2 Hz, 1H), 1.79 (s, 3H). | |
| 28 | ¹H NMR (DMSO-d₆) δ = 8.09 (s, 1H). 7.93 − 7.86 | 313.05 |
| | (m, 2H), 5.44 − 5.42 (m, 1H), 4.63 (s, 3H), 3.90 | |
| | (t, J = 9.1 Hz, 1H), 3.49 (dd, J = 3.5, 9.2 | |
| | Hz, 1H), 1.81 (d, J = 1.8 Hz, 3H). | |
| 29 | ¹H NMR (DMSO-d₆) δ = 8.09 (br. s, 1H), 7.94 − | 313.1 |
| | 7.85 (m, 2H), 5.44 (dd, J = 2.0, 5.2 Hz, 1H), 4.63 | |
| | (s, 3H), 3.90 (t, J = 9.2 Hz, 1H), 3.49 (dd, | |
| | J = 3.6, 9.1 Hz, 1H), 1.81 (d, J = 2.0 Hz, 3H). | |
| 30 | ¹H NMR (DMSO-d₆) δ = 8.09 (br. s, 1H), 7.96 − | 313.14 |
| | 7.81 (m, 2H), 5.44 (dd, J = 2.0, 5.2 Hz, 1H), 4.63 | |
| | (s, 3H), 3.90 (t, J = 9.1 Hz, 1H), 3.49 (dd, | |
| | J = 3.7, 9.2 Hz, 1H), 1.81 (d, J = 1.8 Hz, 3H). | |
| 31 | ¹H NMR (DMSO-d₆) δ = 8.11 (s, 1H), 7.97 (s, 1H), | 342.22 |
| | 7.69 (d, J = 8.6 Hz, 1H), 7.56 (d, J = 8.6 Hz, | |
| | 1H), 5.38 − 5.36 (m, 1H), 4.83 − 4.70 (m, 2H), | |
| | 4.60 − 4.58 (m, 1H), 3.97 − 3.85 (m, 3H), 3.46 | |
| | (dd, J = 3.3, 9.0 Hz, 1H), 1.79 (d, J = 1.8 Hz, 3H). | |
| 32 | ¹H NMR (DMSO-d₆) δ = 8.36 (s, 1H), 7.99 (s, 1H), | 382.1 |
| | 7.71 (d, J = 8.8 Hz, 1H), 7.57 (d, J = 8.4 Hz, | |
| | 1H), 5.44 − 5.42 (m, 1H), 5.29 − 5.25 (m, 1H), | |
| | 4.12 − 4.06 (m, 2H), 3.92 − 3.87 (m, 1H), 3.64 − | |
| | 3.58 (m, 2H), 3.48 (dd, J = 2.9, 9.2 Hz, 1H), | |
| | 2.45 − 2.41 (m, 1H), 2.26 (d, J = 12.5 Hz, | |
| | 1H), 2.16 − 2.07 (m, 2H), 1.76 (d, J = 2.0 Hz, 3H). | |
| 33 | ¹H NMR (DMSO-d₆) δ 8.17 (s, 1H), 8.01 (s, 1H), | 356.2 |
| | 7.70 (d, J = 8.6 Hz, 1H), 7.56 (d, J = 8.6 Hz, | |
| | 1H), 5.44 − 5.30 (m, 1H), 4.90 − 4.77 (m, 1H), | |
| | 4.78 − 4.60 (m, 1H), 3.98 − 3.75 (m, 3H), 3.53 − | |
| | 3.41 (m, 1H), 3.23 (s, 3H), 1.80 (d, J = 2.1 Hz, 3H). | |
| 34 | ¹H NMR (DMSO-d₆) δ = 8.95 − 8.94 (m, 1H), 8.87 | 309.37 |
| | (dd, J = 0.8, 8.3 Hz, 1H), 8.28 (d, J = 8.8 Hz, | |
| | 1H), 8.06 (br. s, 1H), 7.88 (d, J = 9.2 Hz, 1H), | |
| | 7.67 (dd, J = 4.2, 8.3 Hz, 1H), 5.50 − 5.45 | |
| | (d, J = 8.8 Hz, 1H), 3.90 (t, J = 9.0 Hz, 1H), | |
| | 3.49 (dd, J = 2.8, 9.2 Hz, 1H), 1.80 (s, 3H). | |
| 35 | ¹H NMR (Methanol-d₄) δ 8.18 (s, 1H), 7.69 (d, | 297.85 |
| | J = 8.6 Hz, 1H), 7.65 − 7.47 (m, 1H), 5.47 (s, | |
| | 1H), 3.93 (t, J = 9.0 Hz, 1H), 3.58 (dd, J = 9.2, | |
| | 2.7 Hz, 1H), 1.80 (d, J = 2.1 Hz, 3H). | |
| 36 | ¹H NMR (500 MHz, DMSO-d₆) δ = 8.95 − 8.94 (m, | 309.33 |
| | 1H), 8.87 (d, J = 1.0, 8.5 Hz, 1H), 8.28 (d, J = | |
| | 9.0 Hz, 1H), 8.06 (br. s, 1H), 7.88 (d. J = 9.0 Hz, | |
| | 1H), 7.67 (dd, J = 4.0, 8.3 Hz, 1H), 5.48 | |
| | (d, J = 8.8 Hz, 1H), 3.90 (t, J = 8.8 Hz, 1H), | |
| | 3.49 (dd, J = 2.5, 9.0 Hz, 1H), 1.79 (s, 3H). | |
| 37 | ¹H NMR (DMSO-d₆) δ 8.60 (s, 1H), 8.09 (s, 1H), | 369.85 |
| | 7.87 (d, J = 8.6 Hz, 1H), 7.63 (d, J = 8.6 Hz, | |
| | 1H), 5.41 (ddd, J = 9.1, 3.4, 2.1 Hz, 1H), 4.99 | |
| | (d, J = 13.8 Hz, 1H), 4.92 (s, 1H), 4.56 (d, | |
| | J = 13.8 Hz, 1H), 3.98 − 3.77 (m, 1H), 3.56 − 3.42 | |
| | (m, 1H), 1.79 (d, J = 2.1 Hz, 3H), 1.25 (s, | |
| | 3H), 1.07 (s, 3H). | |
| 38 | ¹H NMR (DMSO-d₆) δ = 7.90 (s, 1H), 6.95 (d, J = | 315.42 |
| | 8.5 Hz, 1H), 6.66 (d, J = 8.5 Hz, 1H), 5.47 | |
| | (s, 1H), 5.30 − 5.27 (m, 1H), 4.17 (t, J = 4.4 Hz, | |
| | 2H), 3.84 (t, J = 9.0 Hz, 1H), 3.44 − 3.38 (m, | |
| | 3H), 1.79 (d, J = 2.0 Hz, 3H). | |
| 39 | ¹H NMR (DMSO-d₆) δ = 8.04 (s, 1H), 7.67 (d, J = | 357.34 |
| | 8.3 Hz, 1H), 6.89 (d, J = 8.8 Hz, 1H), 5.37 − | |
| | 5.30 (m, 1H), 4.84 − 4.76 (m, 1H), 4.47 − 4.43 | |
| | (m, 1H), 4.18 − 4.13 (m, 1H), 3.81 (t, J = 9.0 | |
| | Hz, 1H), 3.43 (dd, J = 2.7, 9.0 Hz, 1H), 2.97 − | |
| | 2.82 (m, 1H), 2.20 (s, 3H), 1.78 (d, J = 2.0 Hz, 3H). | |
| 40 | ¹H NMR (DMSO-d₆) δ 7.91 (s, 1H), 7.16 (d, J = | 343.1 |
| | 8.4 Hz, 1H), 6.73 (d, J = 8.4 Hz, 1H), 5.30 | |
| | (dt, J = 8.9, 2.8 Hz, 1H), 4.06 − 3.89 (m, 2H), | |
| | 3.89 − 3.77 (m, 1H), 3.49 − 3.36 (m, 1H), 3.37 − 3. 28 | |
| | (m, 1H), 3.27 (s, 3H), 1.79 (d, J = 2.1 Hz, 3H), 1.14 | |
| | (d, J = 6.6 Hz, 3H). | |
| 41 | ¹H NMR (500 MHz, DMSO-d₆) δ = 7.89 (s, 1H), 7.12 | 359.35 |
| | (d, J = 8.5 Hz, 1H), 6.71 (d, J = 8.5 Hz, 1H), | |
| | 5.28 − 5.23 (m, 1H), 4.55 − 4.52 (m, 1H), 4.14 − | |
| | 4.07 (m, 2H), 3.87 − 3.80 (m, 1H), 3.80 − | |
| | 3.67 (m, 4H), 3.47 − 3.34 (m, 3H), 1.78 (d, J = 2.1 Hz, 3H). | |
| 42 | ¹H NMR (DMSO-d₆) δ = 7.90 (s, 1H), 7.13 (d, J = | 373.43 |
| | 8.3 Hz, 1H), 6.72 (d, J = 8.3 Hz, 1H), 5.25 (d, | |
| | J = 8.8 Hz, 1H), 4.14 − 4.02 (m, 2H), 3.98 − | |
| | 3.75 (m, 310, 3.72 − 3.69 (m, 2H), 3.44 − 3.37 | |
| | (m, 2H), 3.36 − 3.34 (m. 1H), 3.27 (s, 3H), 1.79 | |
| | (d, J = 1.5 Hz, 3H). | |
| 43 | ¹H NMR (DMSO-d₆) δ = 6.76 (t, J = 8.4 Hz, 1H), | 426.2 |
| | 6.51 (t, J = 5.7 Hz, 1H), 6.39 (d, J = 8.5 Hz, | |
| | 1H), 4.37 − 4.31 (m, 2H), 3.93 − 3.83 (m, 3H), | |
| | 3.25 − 3.08 (m, 10H), 1.78 (d, J = 2.2 Hz, 3H), | |
| | 1.48 − 1.41 (m, 2H), 1.38 − 1.30 (m, 4H). | |
| 44 | ¹H NMR (DMSO-d₆) δ = 6.76 (d, J = 8.4 Hz, 1H), | 441.3 |
| | 6.59 (t, J = 5.6 Hz, 1H), 6.39 (d, J = 8.8 Hz, | |
| | 1H), 4.34 (dd, J = 3.1, 5.2 Hz, 2H), 3.92 − 3.81 | |
| | (m, 3H), 3.26 − 3.11 (m, 10H), 2.17 − 2.10 | |
| | (m, 4H), 2.05 (s, 3H), 1.78 (d, J = 2.0 Hz, 3H). | |
| 45 | ¹H NMR (500 MHz, DMSO-d₆) δ = 7.87 (s, 1H), 7.23 | 355.2 |
| | (d, J = 8.2 Hz, 1H), 6.72 (d, J = 8.5 Hz, 1H), | |
| | 5.33 − 5.26 (m, 1H), 4.17 (t, J = 4.6 Hz, 2H), | |
| | 3.82 (t, J = 9.0 Hz, 1H), 3.43 − 3.40 (m, 1H), | |
| | 3.25 − 3.23 (m, 1H), 3.17 (d, J = 5.5 Hz, 1H), | |
| | 2.97 − 2.93 (m, 1H), 1.77 (d, J = 2.1 Hz, 3H), | |
| | 1.13 − 1.05 (m, 1H), 0.86 - 0.78 (m, 1H), 0.74 − 0.67 (m, 2H). | |
| 46 | ¹H NMR (500 MHz, DMSO-d₆) δ = 8.05 (s, 1H), 7.58 | 343.44 |
| | (d, J = 8.5 Hz, 1H), 7.00 (d, J = 8.5 Hz, 1H), | |
| | 5.36 − 5.28 (m, 1H), 4.68 − 4.56 (m, 2H), 3.86 | |
| | (t, J = 9.2 Hz, 1H), 3.75 (s, 3H), 3.45 (dd, | |
| | J = 4.0, 9.2 Hz, 1H), 1.79 (d, J = 1.8 Hz, 3H). | |
| 47 | ¹H NMR (DMSO-d₆) δ = 8.03 (s, 1H), 7.62 (d, J = | 393.3 |
| | 8.3 Hz, 1H), 6.86 (d, J = 8.3 Hz, 1H), 5.32 − | |
| | 5.24 (m, 1H), 4.36 − 4.30 (m, 2H), 3.90 − 3.78 | |
| | (m, 2H), 3.75 (s, 3H), 3.72 − 3.63 (m, 1H), 3.43 | |
| | (dd, J = 2.4, 9.3 Hz, 1H), 1.80 (d, J = 2.0 Hz, 3H). | |
| 48 | ¹H NMR (500 MHz, DMSO-d₆) δ = 7.89 (br. s, 1H), | 342.97 |
| | 7.16 (d, J = 8.2 Hz, 1H), 6.73 (d, J = 8.2 Hz, | |
| | 1H), 5.33 − 5.26 (m, 1H), 4.03 − 3.92 (m, 2H), | |
| | 3.83 (t, J = 9.0 Hz, 1H), 3.41 (dd, J = 3.2, | |
| | 9.0 Hz, 1H), 3.34 − 3.33 (m, 1H), 3.27 (s, 3H), | |
| | 1.79 (d, J = 2.1 Hz, 3H), 1.14 (d, J = 6.7 Hz, 3H). | |
| 49 | ¹H NMR (500 MHz, DMSO-d₆) δ = 7.89 (br. s, 1H), | 342.97 |
| | 7.17 (d, J = 8.5 Hz, 1H), 6.73 (d, J = 8.2 Hz, | |
| | 1H), 5.33 − 5.27 (m, 1H), 4.02 − 3.92 (m, 2H), | |
| | 3.83 (t, J = 9.0 Hz, 1H), 3.41 (dd, J = 3.2, | |
| | 9.0 Hz, 1H), 3.34 − 3.33 (m, 1H), 3.27 (s, 3H), | |
| | 1.79 (d, J = 2.1 Hz, 3H), 1.13 (d, J = 6.5 Hz, 3H). | |
| 50 | ¹H NMR (500 MHz, DMSO-d₆) δ = 13.29 (br. s, 1H), | 298.46 |
| | 8.28 (s, 1H), 8.00 (br. s, 1H), 7.84 (d, J = | |
| | 8.5 Hz, 1H), 7.46 (d, J = 9.0 Hz, 1H), 5.47 − | |
| | 5.44 (m, 1H), 3.85 (t, J = 9.0 Hz, 1H), 3.45 (dd, | |
| | J = 2.5, 9.5 Hz, 1H), 1.78 (d, J = 2.0 Hz, 3H). | |
| 51 | ¹H NMR (500 MHz, DMSO-d₆) δ = 8.62 (s, 1H), 7.98 | 312.3 |
| | (s, 1H), 7.72 (d, J = 9.0 Hz, 1H), 7.48 (d, | |
| | J = 9.0 Hz, 1H), 5.40 (d, J = 9.0 Hz, 1H), 4.20 | |
| | (s, 3H), 3.85 (t, J = 9.0 Hz, 1H), 3.44 | |
| | (dd, J = 2.0, 9.0 Hz, 1H), 1.78 (d, J = 2.0 Hz, 3H). | |
| 52 | ¹H-NMR (DMSO-d₆) δ = 8.82 (s, 1H), 8.09 (s, 1H), | 299.09 |
| | 7.99 (d, J = 8.6 Hz, 1H), 7.71 (d. J = 8.6 Hz, | |
| | 1H), 5.48 − 5.42 (m, 1H), 3.88 (t, J = 9.0 Hz, | |
| | 1H), 3.48 (dd, J = 2.4, 9.3 Hz, 1H), 1.79 (d, J = 2.0 Hz, 3H). | |
| 53 | ¹H-NMR (DMSO-d₆) δ = 8.81 (s, 1H), 8.09 (s, 1H), | 299.05 |
| | 7.99 (d, J = 8.6 Hz, 1H), 7.71 (d, J = 8.8 | |
| | Hz, 1H), 5.47 − 5.43 (m, 1H), 3.88 (t, J = 8.9 Hz, | |
| | 1H), 3.48 (dd, J = 2.3, 9.2 Hz, 1H), 1.80 (d, J = 2.2 Hz, 3H). | |
| 54 | ¹H NMR (Chloroform-d) δ 7.54 - 7.43 (m, 1H), | 328.09 |
| | 6.84 − 6.73 (m, 1H), 5.09 − 4.97 (m, 2H), 4.75 − | |
| | 4.60 (m, 2H), 3.59 − 3.45 (m, 2H), 1.83 (d, J = | |
| | 2.1 Hz, 3H), 1.52 (s, 3H), 1.43 (s, 3H). | |
| 56 | ¹H-NMR (500 MHz, DMS0-d₆) δ = 7.97 (s, 1H), 7.30 | 330.09 |
| | (d, J = 8.5 Hz, 1H), 6.81 (d, J = 8.5 Hz, 1H), | |
| | 5.31 (dd, J = 1.8, 7.6 Hz, 1H), 4.43 − 4.27 | |
| | (m, 1H), 4.09 − 4.02 (m, 1H), 1.81 (d, J = 2.1 | |
| | Hz, 3H), 1.29 (d, J = 6.4 Hz, 3H). | |
| 57 | ¹H-NMR (500 MHz, DMSO-d₆) δ = 7.96 (s, 1H), 7.56 | 328.2 |
| | (d, J = 8.5 Hz, 1H), 6.70 (d. J = 8.9 Hz, 1H), | |
| | 5.33 (dd, J = 2.0, 7.5 Hz, 1H), 4.21 − 4.11 | |
| | (m, 2H), 4.10 − 4.02 (m, 1H), 3.03 − 2.91 (m, 2H), | |
| | 1.99 (quin, J = 5.8 Hz, 2H), 1.81 (d, J = 2.1 | |
| | Hz, 3H), 1.29 (d, J = 6.4 Hz, 3H). | |
| 58 | ¹H-NMR (DMSO-d₆) δ = 7.95 (s, 1H), 7.29 (d, J = | 316.2 |
| | 8.5 Hz, 1H), 6.81 (d, J = 8.5 Hz, 1H), 5.32 − 5.28 | |
| | (m, 1H), 4.41 − 4.27 (m, 4H), 3.80 (t, J = 9.2 | |
| | Hz, 1H), 3.42 − 3.38 (m, 1H), 1.78 (d, J = 2.4 Hz, 3H). | |
| 59 | ¹H-NMR (Chloroform-d) δ = 7.19 (d, J =8.5 Hz, | 316.2 |
| | 1H), 6.86 (d, J = 8.5 Hz, 1H), 5.52 − 5.46 (m, 1H), | |
| | 5.42 5.33 (m, 1H), 4.48 4.41 (m, 2H), 4.39 | |
| | 4.29 (m, 2H), 3.91 − 3.84 (m, 1H), 3.67 − 3.61 (m, 1H), | |
| | 1.80 (d. J = 1.8 Hz, 3H). | |
| 60 | ¹H NMR (DMSO-d₆) δ = 8.09 (s, 1H), 7.61 (d, J = | 330.18 |
| | 8.4 Hz, 1H), 6.76 (d, J = 8.5 Hz, 1H), 5.37 (dd, | |
| | J = 2.3, 8.0 Hz, 1H), 4.98 (brs, 1H), 4.62 | |
| | (t, J = 8.8 Hz, 2H), 4.01 − 3.93 (m, 1H), 3.76 | |
| | (dd, J = 5.8, 10.8 Hz, 1H), 3.63 (dd, J = 7.3, | |
| | 10.8 Hz, 1H), 3.38 (t, J = 8.8 Hz, 2H), 1.79 (d, J = 2.0 Hz, 3H). | |
| 61 | ¹H NMR (DMSO-d₆) δ = 8.16 (brs, 1H), 7.60 (d, | 330.18 |
| | J = 8.5 Hz, 1H), 6.76 (d, J = 8.3 Hz, 1H), 5.19 | |
| | (br. s, 1H), 5.13 − 5.06 (m, 1H), 4.62 (t, J = | |
| | 8.8 Hz, 2H), 3.75 − 3.66 (m, 1H), 3.49 − 3.43 | |
| | (m, 1H), 3.42 − 3.35 (m, 3H), 1.80 (d, J = 2.0 Hz, 3H). | |
| 62 | ¹H-NMR (DMSO-d₆) δ = 8.03 (s, 1H), 7.36 (d, J = | 304.2 |
| | 8.3 Hz, 1H), 6.93 (d, J = 8.3 Hz, 1H), 5.33 − 5.29 | |
| | (m, 1H), 3.85 − 3.79 (m, 1H), 3.46 − 3.38 (m, 1H), | |
| | 1.79 (d, J = 1.8 Hz, 3H). | |
| 63 | ¹H-NMR (DMSO-d₆) δ = 8.17 (br. s, 1H), 7.60 (d, J = | 329 |
| | 8.4 Hz, 1H), 6.76 (d, J = 8.5 Hz, 1H), 5.12 | |
| | (br. s, 1H), 4.62 (t, J = 8.8 Hz, 2H), 3.64 − | |
| | 3.59 (m, 1H), 3.38 (t, J = 8.8 Hz, 2H), 3.29 − | |
| | 3.28 (m, 1H), 2.68 − 2.55 (m, 2H), 1.80 (d, J = 2.3 Hz, 3H). | |
| 64 | ¹H-NMR (DMSO-d₆) δ = 8.12 (br. s, 1H), 7.30 (d, | 346.18 |
| | J = 8.8 Hz, 1H), 6.82 (d, J = 8.5 Hz, 1H), 5.33 | |
| | (d, J = 6.0 Hz, 1H), 5.12 − 5.00 (m, 1H), 4.43 − | |
| | 4.26 (m, 4H), 4.01 − 3.93 (m, 1H), 3.76 (dd, | |
| | J = 5.6, 10.4 Hz, 1H), 3.67 − 3.60 (m, 1H), 1.80 (s, 3H). | |
| 65 | ¹H-NMR (DMSO-d₆) δ = 8.13 (br. s, 1H), 7.30 (d, | 346.14 |
| | J = 8.6 Hz, 1H), 6.81 (d, J = 8.6 Hz, 1H), 5.18 | |
| | (t, J = 5.6 Hz, 1H), 5.11 (t, J = 2.0 Hz, 1H), | |
| | 4.44 − 4.31 (m, 1H), 4.31 − 4.23 (m, 3H), 3.73 − | |
| | 3.63 (m, 1H), 3.51 − 3.41 (m, 1H), 3.39 − | |
| | 3.33 (m, 1H), 1.81 (d, J = 2.0 Hz, 3H). | |
| 66 | ¹H-NMR (DMSO-d₆) δ = 8.13 (br. s, 1H), 7.30 (d, | 346.22 |
| | J = 8.6 Hz, 1H), 6.81 (d, J = 8.6 Hz, 1H), 5.18 | |
| | (br. s, 1H), 5.11 (t, J = 2.1 Hz, 1H), 4.44 − | |
| | 4.38 (m, 1H), 4.35 − 4.26 (m, 3H), 3.70 − 3.67 | |
| | (m, 1H), 3. 16 (d, J = 9.3 Hz, 1H), 3.39 − 3.34 | |
| | (m, 1H), 1.81 (d, J = 2.9 Hz, 3H). | |
| 67 | ¹H-NMR (DMSO-d₆) δ = 8.37 (br. s, 1H), 7.60 (d, | 342.25 |
| | J = 8.3 Hz, 1H), 6.76 (d, J = 8.3 Hz, 1H), 4.94 | |
| | (br. s, 1H), 4.62 (t, J = 8.8 Hz, 2H), 3.54 | |
| | (dd, J = 2.8, 5.3 Hz, 1H), 3.45 − 3.36 (m, 2H), | |
| | 1.85 − 1.76 (m, 4H), 0.87 (dd, J = 6.8, 19.3 Hz, 6H). | |
| 68 | ¹H-NMR (DMSO-d₆) δ = 8.12 (br. s, 1H), 7.61 (d, | 342.25 |
| | J = 8.3 Hz, 1H), 6.76 (d, J = 8.3 Hz, 1H), 5.34 − | |
| | 5.27 (m, 1H), 4.63 (t, J = 8.8 Hz, 2H), 3.52 | |
| | (dd, J = 7.2, 9.9 Hz, 1H), 3.39 (t, J = 8.9 | |
| | Hz, 2H), 2.05 − 1.93 (m, 1H), 1.79 (d, J = 2.0 | |
| | Hz, 3H), 1.02 (d, J = 6. 6 Hz, 3H), 0.97 ( d, J = 6.6 Hz, 3H). | |
| 69 | ¹H-NMR (DMSO-d₆) δ = 8.24 (br. s, 1H), 7.61 (d, | 357.3 |
| | J = 8.3 Hz, 1H), 6.76 (d, J = 8.5 Hz, 1H), 5.05 | |
| | (br. s, 1H), 4.62 (t, J = 8.8 Hz, 2H), 3.81 | |
| | (t, J = 5.9 Hz, 1H), 3.42 − 3.36 (m, 2H), 2.43 − | |
| | 2.35 (m, 1H), 2.28 (dd, J = 5.8, 12.3 Hz, 1H), | |
| | 2.21 (s, 6H), 1.80 (d, J = 1.8 Hz, 3H). | |
| 70 | ¹H-NMR (DMSO-d₆) δ = 8.23 (br. s, 1H), 7.61 (d, | 357.27 |
| | J = 8.5 Hz, 1H), 6.76 (d, J = 8.5 Hz, 1H), 5.05 | |
| | (br. s, 1H), 4.63 (t, J = 8.8 Hz, 2H), 3.84 − | |
| | 3.78 (m, 1H), 3.39 (dt, J = 2.5, 8.8 Hz, 2H), | |
| | 2.43 − 2.35 (m, 1H), 2.28 (dd, J = 5.6, 12.4 Hz, | |
| | 1H), 2.21 (s, 6H) 1.80 (d, J = 1.8 Hz, 3H). | |
| 71 | ¹H-NMR (DMSO-d₆) δ = 8.31 (br. s, 1H), 7.59 (d, | 328.74 |
| | J = 8.3 Hz, 1H), 6.74 (d, J = 8.3 Hz, 1H), 4.93 − | |
| | 4.86 (m, 1H), 4.60 (t, J = 8.8 Hz, 2H), 3.62 | |
| | (dt, J = 2.8, 6.3 Hz, 1H), 3.40 − 3.33 (m, 2H), | |
| | 1.78 (d, J = 1.8 Hz, 3H), 1.67 − 1.46 (m, 2H), | |
| | 0.88 (t, J = 7.4 Hz, 3H). | |
| 72 | ¹H-NMR (DMSO-d₆) δ = 8.33 (br. s, 1H), 7.60 (d, | 328.31 |
| | J = 8.4 Hz, 1H), 6.76 (d, J = 8.5 Hz, 1H), 4.94 − | |
| | 4.89 (m, 1H), 4.62 (t, J = 8.9 Hz, 2H), 3.64 | |
| | (dt, J = 2.8, 6.3 Hz, 1H), 3.42 − 3.35 (m, 2H), | |
| | 1.80 (d, J = 2.0 Hz, 3H), 1.67 − 1.47 (m, 2H), | |
| | 0.90 (t, J = 7.4 Hz, 3H). | |
| 73 | ¹H-NMR (DMSO-d₆) δ = 8.12 (br. s, 1H), 7.60 (d, | 328.31 |
| | J = 2.4 Hz, 1H), 6.76 (d, J = 8.5 Hz, 1H), 5.35 | |
| | (dd, J = 2.0, 7.5 Hz, 1H), 4.63 (t, J = 8.8 | |
| | Hz, 2H), 3.83 (q, J = 7.0 Hz, 1H), 3.38 (t, J = | |
| | 8.8 Hz, 2H), 1.80 (d, J = 2.0 Hz, 3H), 1.71 (qd, | |
| | J = 6.9, 17.6 Hz. 2H), 0.98 (t, J = 7.4 Hz, 3H). | |
| 74 | ¹H-NMR (DMSO-d₆) δ = 8.20 (br. s, 1H), 7.61 (d, | 344.30 |
| | J = 8.3 Hz, 1H), 6.76 (d, J = 8.3 Hz, 1H), 5.39 − | |
| | 5.37 (m, 1H), 4.63 (t, J = 8.8 Hz, 2H), 4.16 − | |
| | 4.06 (m, 1H), 3.66 (dd, J = 5.5, 9.7 Hz, 1H), | |
| | 3.59 − 3.53 (m, 1H), 3.40 − 3.33 (m, 5H), | |
| | 1.81 (d, J = 2.0 Hz, 3H). | |
| 75 | ¹H-NMR (DMSO-d₆) δ = 8.22 (br. s, 1H), 7.61 (d, | 344.27 |
| | J = 8.4 Hz. 1H), 6.76 (d, J = 8.3 Hz, 1H), 5.38 | |
| | (dd, J = 2.2, 8.1 Hz. 1H), 4.63 (t, J = 8.8 | |
| | Hz, 2H), 4.11 (dt, J = 5.7, 7.4 Hz, 1H), 3.69 − | |
| | 3.63 (m, 1H), 3.60 − 3.54 (m, 1H), 3.41 − 3.37 | |
| | (m, 3H), 2.04 − 1.99 (m, 1H), 1.81 (d, J = 2.2 | |
| | Hz, 3H), 1.57 − 1.52 (m, 1H). | |
| 76 | ¹H-NMR (DMSO-d₆) δ = 8.27 (br. s, 1H), 7.61 (d, | 344.34 |
| | J = 8.6 Hz, 1H), 6.76 (d, J = 8.6 Hz, 1H), | |
| | 5.05 (t, J = 2.2 Hz, 1H), 4.62 (t, J = 8.9 Hz, 2H), | |
| | 3.87 − 3.84 (m, 1H), 3.46 − 3.37 (m, 4H), 3.28 | |
| | (s, 3H), 1.80 (d, J = 2.0 Hz, 3H). | |
| 77 | ¹H-NMR (DMSO-d₆) δ = 8.27 (br. s, 1H), 7.61 (d, | 344.30 |
| | J = 8.4 Hz, 1H), 6.76 (d, J = 8.3 Hz, 1H), 5.05 | |
| | (t, J = 2.2 Hz, 1H), 4.62 (t, J = 8.8 Hz, 2H), | |
| | 3.87 − 3.84 (m, 1H), 3.46 − 3.37 (m, 4H), 3.28 | |
| | (s, 3H), 1.80 (d, J = 2.2 Hz, 3H). | |
| 78 | ¹H NMR (DMSO-d₆) δ = 8.43 (br. s, 1H), 7.62 (d, | 331.9 |
| | J = 8.5 Hz, 1H), 6.77 (d, J = 8.5 Hz, 1H), | |
| | 5.19 (br. s, 1H), 4.66 − 4.36 (m, 4H), 4.05 − 3.96 | |
| | (m, 1H), 3.39 (t, J = 8.4 Hz, 2H), 1.81 (d, J = 1.5 Hz, 3H). | |
| 79 | ¹H NMR (DMSO-d₆) δ = 8.42 (br. s, 1H), 7.62 (d, | 332.16 |
| | J = 8.4 Hz, 1H), 6.77 (d, J = 8.3 Hz, 1H), | |
| | 5.21 − 5.15 (m, 1H), 4.66 − 4.39 (m, 4H), 4.07 − | |
| | 3.96 (m, 1H), 3.39 (t, J = 8.8 Hz, 2H), 1.81 (d, J = 2.0 Hz, 3H). | |
| 80 | ¹H NMR (DMSO-d₆) δ = 8.40 (br. s, 1H), 7.62 (d, | 332.16 |
| | J = 8.5 Hz, 1H), 6.77 (d, J = 8.5 Hz, 1H), 5.47 | |
| | (d, J = 6.8 Hz, 1H), 4.86 − 4.53 (m, 4H), 4.34 − | |
| | 4.19 (m, 1H), 3.41 − 3.35 (m, 2H), 1.81 (d, J = 1.5 Hz, 3H). | |
| 81 | ¹H NMR (DMSO-d₆) δ = 8.28 (br. s, 1H), 7.60 (d, | 344.16 |
| | J = 8.5 Hz, 1H), 6.75 (d, J = 8.3 Hz, 1H), 5.19 − | |
| | 5.00 (m, 2H), 4.62 (t, J = 8.8 Hz, 2H), 3.76 − | |
| | 3.67 (m, 1H), 3.61 (t, J = 3.0 Hz, 1H), 3.38 | |
| | (dd, J = 8.0, 9.8 Hz, 2H), 1.80 (d, J = 2.0 | |
| | Hz, 3H), 1.05 (d, J = 6.5 Hz, 3H). | |
| 82 | ¹H NMR (DMSO-d₆) δ = 8.01 (br. s, 1H), 7.61 (d, | 344.16 |
| | J = 8.3 Hz, 1H), 6.76 (d, J = 8.3 Hz, 1H), 5.33 − | |
| | 5.26 (m, 1H), 5.00 (br. s, 1H), 4.63 (t, J = | |
| | 8.9 Hz, 2H), 3.93 − 3.81 (m, 1H), 3.66 (dd, | |
| | J = 7.6, 8.9 Hz, 1H), 3.38 (t, J = 8.8 Hz, 2H), | |
| | 1.78 (d, J = 2.0 Hz, 3H), 1.19 (d, J = 6.3 Hz, 3H). | |
| 83 | ¹H NMR (DMSO-d₆) δ = 8.27 (br. s, 1H), 7.60 (d, | 344.2 |
| | J = 8. 5 Hz, 1H), 6.76 (d, J = 8.3 Hz, 1H), 5.17 − | |
| | 5.15 (m, 2H), 4.62 (t, J = 8.8 Hz, 2H), 3.54 − | |
| | 3.46 (m, 2H), 3.39 (dd, J = 8.1, 10.1 Hz, | |
| | 2H), 1.79 (d, J = 1.8 Hz, 3H), 1.10 (d, J = 6.0 Hz, 3H). | |
| 84 | ¹H NMR (DMSO-d₆) δ = 7.99 (br. s, 1H), 7.61 (d, | 344.23 |
| | J = 8.3 Hz, 1H), 6.76 (d, J = 8.5 Hz, 1H), 5.31 − | |
| | 5.25 (m, 1H), 4.92 (br. s, 1H), 4.63 (t, J = | |
| | 8.8 Hz, 2H), 4.00 − 3.90 (m, 1H), 3.67 − 3.60 | |
| | (m, 1H), 3.39 (t, J = 8.8 Hz, 2H), 1.79 (d, J = | |
| | 1.8 Hz, 3H), 1.20 (d, J = 6.4 Hz, 3H). | |

TABLE 6

| | | Retention |
|---|---|---|
| Example | | Time |
| No. | Analytical Condition | (min.) |
| 9 | Supercritical fluid chromatography | 2.299 |
| | Column: Chiralpak IG-3 | |
| | Mobile Phase: Carbon dioxide/methanol | |
| 10 | Supercritical fluid chromatography | 3.443 |
| | Column: Chiralpak IG-3 | |
| | Mobile Phase: Carbon dioxide/methanol | |
| 13 | Supercritical fluid chromatography | 3.393 |
| | Column: Chiralpak IG-3 | |
| | Mobile Phase: Carbon dioxide/methanol | |
| 14 | Supercritical fluid chromatography | 4.294 |
| | Column: Chiralpak IG-3 | |
| | Mobile Phase: Carbon dioxide/methanol | |
| 16 | Supercritical fluid chromatography | 2.552 |
| | Column: Chiralpak IE-3 | |
| | Mobile Phase: Carbon dioxide/methanol | |
| 17 | Supercritical fluid chromatography | 3.819 |
| | Column: Chiralpak IE-3 | |
| | Mobile Phase: Carbon dioxide/methanol | |
| 29 | Supercritical fluid chromatography | 4.612 |
| | Column: Chiralpak IG-3 | |
| | Mobile Phase: Carbon dioxide/isopropanol | |
| 30 | Supercritical fluid chromatography | 6.063 |
| | Column: Chiralpak IG-3 | |
| | Mobile Phase: Carbon dioxide/isopropanol | |
| 34 | Supercritical fluid chromatography | 4.81 |
| | Column: Lux Amylose-2 | |
| | Mobile Phase: Carbon dioxide/methanol | |
| 36 | Supercritical fluid chromatography | 5.73 |
| | Column: Lux Amylose-2 | |
| | Mobile Phase: Carbon dioxide/methanol | |
| 48 | Supercritical fluid chromatography | 3.135 |
| | Column: Chiralpak IC-3 | |
| | Mobile Phase: Carbon dioxide/ | |
| | 0.5% diethyamine-methanol | |
| 49 | Supercritical fluid chromatography | 4.091 |
| | Column: Chiralpak IC-3 | |
| | Mobile Phase: Carbon dioxide/ | |
| | 0.5% diethyamine-methanol | |
| 52 | Supercritical fluid chromatography | 3.526 |
| | Column: Chiralpak AD-3 | |
| | Mobile Phase: Carbon dioxide/methanol | |
| 53 | Supercritical fluid chromatography | 5.924 |
| | Column: Chiralpak AD-3 | |
| | Mobile Phase: Carbon dioxide/methanol | |
| 56 | Supercritical fluid chromatography | 5.428 |
| | Column: Chiralcel OD-3 | |
| | Mobile Phase: Carbon dioxide/ | |
| | 0.5% diethyamine-methanol | |
| 57 | Supercritical fluid chromatography | 4.176 |
| | Column: Chiralpak IC-3 | |
| | Mobile Phase: Carbon dioxide/ | |
| | 0.5% diethyamine-methanol | |
| 58 | HPLC | 8.28 |
| | Column: Chiralpak IC | |
| | Mobile Phase: 0.1% diethylamine- | |
| | Chloroform/0.1% diethylamine-methanol | |
| 59 | HPLC | 12.4 |
| | Column: Chiralpak IC | |
| | Mobile Phase: 0.1% diethylamine- | |
| | Chloroform/0.1% diethylamine-methanol | |
| 60 | Supercritical fluid chromatography | 5.31 |
| | Column: DCPAK SFC-B | |
| | Mobile Phase: Carbon dioxide/methanol | |
| 61 | Supercritical fluid chromatography | 6.447 |
| | Column: DCPAK SFC-B | |
| | Mobile Phase: Carbon dioxide/methanol | |
| 64 | Supercritical fluid chromatography | 4.328 |
| | Column: Chiralpak IG | |
| | Mobile Phase: Carbon dioxide/ | |
| | 0.5% diethyamine-methanol | |
| 65 | Supercritical fluid chromatography | 2.818 |
| | Column: Chiralpak IG | |
| | Mobile Phase: Carbon dioxide/ | |
| | 0.5% diethyamine-methanol | |
| 66 | Supercritical fluid chromatography | 3.384 |
| | Column: Chiralpak IG | |
| | Mobile Phase: Carbon dioxide/ | |
| | 0.5% diethyamine-methanol | |
| 67 | Supercritical fluid chromatography | 4.64 |
| | Column: Chiralcel OX-H | |
| | Mobile Phase: Carbon dioxide/methanol | |
| 68 | Supercritical fluid chromatography | 10.79 |
| | Column: Chiralcel OX-H | |
| | Mobile Phase: Carbon dioxide/methanol | |
| 69 | Supercritical fluid chromatography | 3.68 |
| | Column: Chiralpak IG | |
| | Mobile Phase: Carbon dioxide/methanol | |
| 70 | Supercritical fluid chromatography | 4.91 |
| | Column: Chiralpak IG | |
| | Mobile Phase: Carbon dioxide/methanol | |
| 71 | Supercritical fluid chromatography | 4.94 |
| | Column: Chiralpak IF | |
| | Mobile Phase: Carbon dioxide/methanol | |
| 72 | Supercritical fluid chromatography | 5.83 |
| | Column: Chiralpak IF | |
| | Mobile Phase: Carbon dioxide/methanol | |
| 73 | Supercritical fluid chromatography | 6.96 |
| | Column: Chiralpak IF | |
| | Mobile Phase: Carbon dioxide/methanol | |
| 74 | Supercritical fluid chromatography | 3.218 |
| | Column: Chiralpak IG | |
| | Mobile Phase: Carbon dioxide/methanol | |
| 75 | Supercritical fluid chromatography | 4.638 |
| | Column: Chiralpak IG | |
| | Mobile Phase: Carbon dioxide/methanol | |
| 76 | Supercritical fluid chromatography | 1.752 |
| | Column: Chiralpak IG | |
| | Mobile Phase: Carbon dioxide/methanol | |
| 77 | Supercritical fluid chromatography | 2.748 |
| | Column: Chiralpak IG | |
| | Mobile Phase: Carbon dioxide/methanol | |
| 78 | Supercritical fluid chromatography | 2.976 |
| | Column: Chiralpak IE-3 | |
| | Mobile Phase: Carbon dioxide/ | |
| | 0.5% diethyamine-methanol | |
| 79 | Supercritical fluid chromatography | 3.537 |
| | Column: Chiralpak IE-3 | |
| | Mobile Phase: Carbon dioxide/ | |
| | 0.5% diethyamine-methanol | |
| 80 | Supercritical fluid chromatography | 4.94 |
| | Column: Chiralpak IE-3 | |
| | Mobile Phase: Carbon dioxide/ | |
| | 0.5% diethyamine-methanol | |
| 81 | Supercritical fluid chromatography | 3.13 |
| | Column: Chiralpak AD-H | |
| | Mobile Phase: Carbon dioxide/ | |
| | 0.5% diethyamine-methanol | |
| 82 | Supercritical fluid chromatography | 2.17 |
| | Column: Chiralpak AD-H | |
| | Mobile Phase: Carbon dioxide/ | |
| | 0.5% diethyamine-methanol | |
| 83 | Supercritical fluid chromatography | 1.92 |
| | Column: Chiralpak IC | |
| | Mobile Phase: Carbon dioxide/ | |
| | 0.5% diethyamine-methanol | |
| 84 | Supercritical fluid chromatography | 2.47 |
| | Column: Chiralpak IC | |
| | Mobile Phase: Carbon dioxide/ | |
| | 0.5% diethyamine-methanol | |

### Test Example 1

[Inhibition Test on Activity of DYRK Family (DYRK1A, DYRK1B, DYRK2, and DYRK2)]

(Method for Measuring Kinase Activity)

The kinase activity was measured by mobility shift assay (MSA) method using QuickScout Screening Assist™ MSA (commercially available kit manufactured by Carna Biosciences, Inc.). The substrate of the kinase reaction used was an FITC-labeled DYRKtide peptide included in the kit. An assay buffer [20 mM HEPES, 0.01% Triton X-100™, 2 mM dithiothreitol, pH 7.5] was used to create a substrate mixture solution with a substrate (4 μM), MgCl₂ (20 mM), and ATP (DYRK1A: 100 μM; DYRK1B: 200 μM; DYRK2: 40 μM; and DYRK3: 20 μM). In addition, kinases (DYRK1A: manufactured by Carna Biosciences, Inc., Cat. No. 04-130; DYRK1B: manufactured by Carna Biosciences, Inc., Cat. No. 04-131; DYRK2; manufactured by Carna Biosciences, Inc., Cat. No. 04-132; and DYRK3; manufactured by Carna Biosciences, Inc., Cat. No. 04-133) were diluted with the assay buffer to prepare enzyme solutions (DYRK1A: 0.2 ng/μL; DYRK1B: 0.08 ng/μL; DYRK2: 0.04 ng/μL; and DYRK3: 0.25 ng/μL). The 10 mM solution of the test compound in DMSO was further diluted with DMSO to 10 levels of the concentration (0.00003 mM, 0.0001 mM, 0.0003 mM, 0.001 mM, 0.003 mM, 0.01 mM, 0.03 mM, 0.1 mM, 0.3 mM, and 1 mM), each of which was subjected to 25-fold dilution with the assay buffer to obtain a drug solution (4% DMSO solution). 5 μL of the drug solution or a control solution (4% DMSO-assay buffer), 5 μL of the substrate mixture solution, and 10 μL of the enzyme solution were mixed in the wells of a polypropylene 384-well plate and allowed to react at room temperature for 1 hour, and then the reaction was quenched by adding 60 μL of the termination buffer included in the kit. Subsequently, the quantities of the substrates (S) and the phosphorylated substrate (P) in the reaction solution were measured using LabChip EZ Reader II system (manufactured by Caliper Life Sciences) according to the protocol of the assay kit.

### (Method for Evaluating Inhibitory Activity)

The heights of the peaks of the “substrate” and the “phosphorylated substrate” were expressed as S and P, respectively, and a blank containing the assay buffer instead of the enzyme solution was also measured.

The inhibition rate (%) of the test compound was calculated according to the following equation:

Inhibition rate (%)=(1−(C−A)/(B−A))×100

wherein, A, B, and C represent P/(P+S) of the blank well, P/(P+S) of the control solution well, and P/(P+S) of the compound-containing well, respectively.

The IC₅₀ value was calculated via a regression analysis of the inhibition rate and the test compound concentration (logarithmic value).

(Evaluation Result)

The inhibiting activities of representative compounds of the present invention are shown against DYRK1A, DYRK1B, DYRK2, and DYRK3 in Tables 7 and 8. The kinase activity inhibitory effect was indicated with the mark *** at an IC₅₀ value of less than 0.01 μM; the mark ** at 0.01 μM or more and less than 0.1 μM; the mark * at 0.1 μM or more and less than 1 μM; and the mark — at 1 μM or more (N.D. indicates not measured).

TABLE 7

| | |
|---|---|
| Test Compound | Inhibitory Activity |

| Example Number | 1a | 1b | 2 | 3 |
|---|---|---|---|---|
| 1 | * * * | * * * | * | * * |
| 2 | * * * | * * * | * * | * * |
| 3 | * * | * * | — | — |
| 4 | — | N.D. | — | — |
| 5 | * * * | * * * | * | * |
| 6 | * * * | N.D. | * | * |
| 7 | * * * | N.D. | — | — |
| 8 | * * * | * * * | * | * * |
| 9 | * * * | N.D. | * * | * * |
| 10 | * | N.D. | — | — |
| 11 | * * | N.D. | * | * |
| 12 | * * * | N.D. | * * | * * |
| 13 | * * * | N.D. | * * | * * |
| 14 | * * | N.D. | * | * |
| 15 | * | N.D. | — | — |
| 16 | * * * | N.D. | — | * |
| 17 | * | N.D. | — | — |
| 18 | * * | N.D. | — | * |
| 19 | * * * | N.D. | * | * |
| 20 | * | N.D. | — | — |
| 21 | * * * | N.D. | — | * |
| 22 | * * | N.D. | — | — |

TABLE 8

| | |
|---|---|
| Test Compound | Inhibitory Activity |

| Example No. | 1a | 1b | 2 | 3 |
|---|---|---|---|---|
| 23 | * * * | N.D. | * | * |
| 24 | * * * | * * * | * | * |
| 25 | * * * | * * * | * | * |
| 26 | * * * | * * * | * | * * |
| 27 | * ** | * * * | * | * * |
| 28 | * * * | * * * | — | * |
| 29 | * | * | — | — |
| 30 | * * * | * * * | * | * |
| 31 | * * * | * * * | * | * |
| 32 | * * * | * * * | * * | * * |
| 33 | * * | * * | * | * |
| 34 | * * * | N.D. | * * | * * |
| 35 | ** * | N.D. | * | * |
| 36 | * * | N.D. | * | * |
| 37 | * * | N.D. | * | * * |
| 38 | * * * | * * * | * | * |
| 39 | * | — | — | — |
| 40 | * * * | N.D. | * | * |
| 41 | * * * | N.D. | * | * |
| 42 | * * * | N.D. | — | * |
| 43 | * | N.D. | — | — |
| 44 | — | N.D. | — | — |
| 45 | * * | N.D. | * | * |
| 46 | * * | N.D. | — | * |
| 47 | * * | N.D. | — | * |
| 48 | * | N.D. | — | — |
| 49 | * * * | N.D. | — | * |
| 50 | * * | N.D. | — | — |
| 51 | * * * | N.D. | * | * |
| 52 | * * * | N.D. | — | * |
| 53 | * | N.D. | — | — |
| 54 | * * | N.D. | — | — |
| 55 | * * * | N.D. | * | * |
| 56 | * * * | N.D. | * | * |
| 57 | * * * | N.D. | * | * |
| 58 | * * * | N.D. | * | * * |
| 59 | — | N.D. | — | — |
| 60 | * * * | N.D. | * | * * |
| 61 | * * * | N.D. | * | * |
| 62 | * * * | N.D. | * | * * |
| 63 | * * * | N.D. | * | * * |
| 64 | * * * | N.D. | * | * |
| 65 | * * * | N.D. | * | * |
| 66 | * | N.D. | — | — |
| 67 | — | N.D. | — | — |
| 68 | * * * | N.D. | — | * |
| 69 | * * | N.D. | — | * |
| 70 | * | N.D. | — | — |
| 71 | * * * | N.D. | — | * |
| 72 | * | N.D. | — | — |
| 73 | * * * | N.D. | * | * |
| 74 | * * * | N.D. | * | * |
| 75 | * | N.D. | — | — |
| 76 | * * | N.D. | — | — |
| 77 | * * | N.D. | — | — |
| 78 | *** | N.D. | * * | * |
| 79 | * | N.D. | — | — |
| 80 | *** | N.D. | — | * |
| 81 | * | N.D. | — | — |
| 82 | * * * | N.D. | * | * * |
| 83 | ** | N.D. | — | — |
| 84 | * * * | N.D. | * | * |

These results have shown that the test compounds (the compounds of the present invention) have potent DYRK-inhibitory activities.

### INDUSTRIAL APPLICABILITY

The compound provided by the present invention is useful as a prophylactic or therapeutic agent for disease which is known to be involved in abnormal cell response through DYRK1A, for example, Alzheimer's disease, Parkinson's disease, Down's syndrome, mental retardation, memory impairment, memory loss, neuropsychiatric disorder such as depression, and cancers such as brain tumors. The compound is a DYRK1B inhibitor also useful as a prophylactic or therapeutic pharmaceutical (pharmaceutical composition) for cancers such as pancreatic cancer. Since DYRK2 controls p53 to induce apoptosis in response to DNA damages, the compound provided by the present invention is further useful as a prophylactic or therapeutic pharmaceutical (pharmaceutical composition) for bone resorption disease and osteoporosis. The compound provided by the present invention is a DYRK3 inhibitor also useful as a prophylactic or therapeutic pharmaceutical (pharmaceutical composition) for sickle-cell anemia, bone resorption disease in chronic kidney disease, and osteoporosis. The compound is also useful, as a compound inhibiting DYRK, for reagents to be used in pathological imaging and for reagents for basic experiments and research regarding the above diseases.

1. An alkyne derivative of the following formula (I): 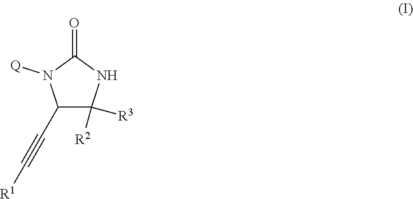
wherein R¹ represents a hydrogen atom, a halogen atom, a trimethylsilyl group, an optionally substituted aryl group, an optionally substituted heteroaryl group, an optionally substituted lower alkyl group, and an optionally substituted cycloalkyl group;
R² and R³ represent each independently a hydrogen atom, a halogen atom, an optionally substituted lower alkyl group, an optionally substituted cycloalkyl group, an optionally substituted aryl group, an optionally substituted heteroaryl group, an optionally substituted saturated heterocyclic group, an optionally substituted heterocyclic fused ring, an optionally substituted alkoxy group, an optionally substituted amino group, an optionally substituted alkynyl group, an optionally substituted alkenyl group, an optionally substituted alkylcarbonyl group, a carboxy group, an alkoxycarbonyl group, an azido group, a nitrile group, an optionally substituted carbamoyl group, an optionally substituted thioether group, an optionally substituted alkylsulfonyl group, an optionally substituted sulfonamide group, a nitro group, or a formyl group; and
Q indicates a structure selected from the following structures (a) to (o): 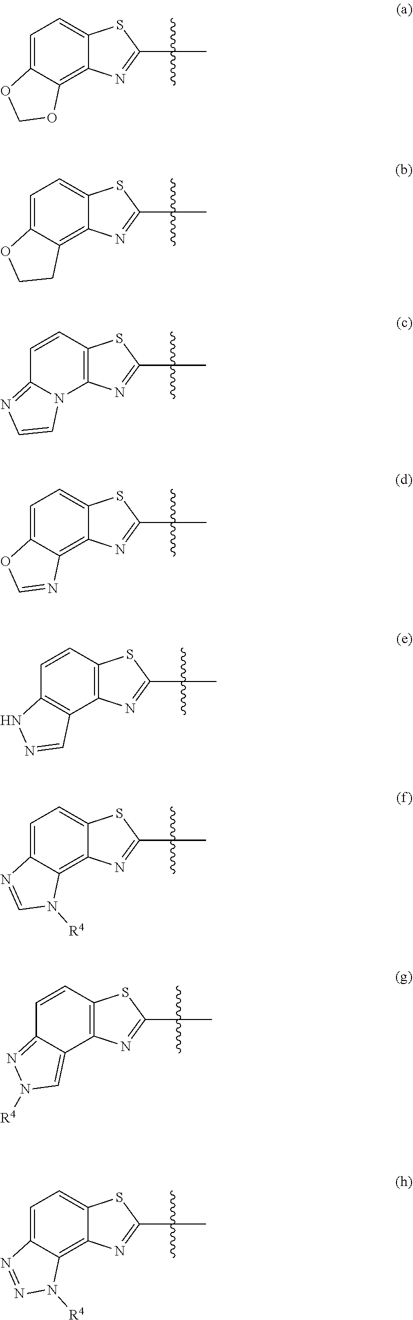 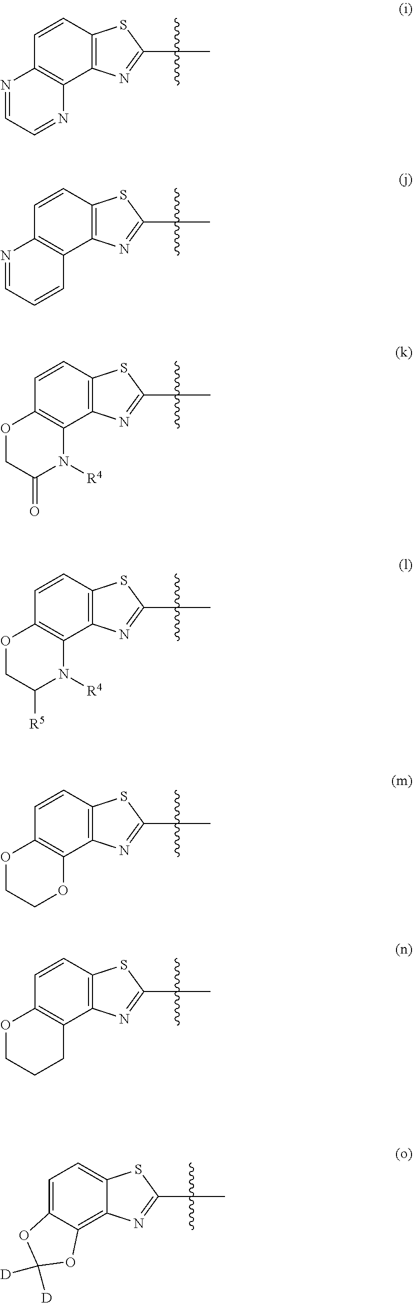
wherein R⁴ represents a hydrogen atom, an optionally substituted lower alkyl group, an optionally substituted cycloalkyl group, an optionally substituted alkylcarbonyl group, an optionally substituted alkylsulfonyl group, or an optionally substituted saturated heterocyclic group; and
R⁵ represents a hydrogen atom or an optionally substituted lower alkyl group, or a pharmaceutically acceptable salt thereof.

2. The alkyne derivative according to claim 1 above or a pharmaceutically acceptable salt thereof, wherein Q is selected from the structures (a) to (d) and (m) in formula (I) above.

3. The alkyne derivative according to claim 2 above or a pharmaceutically acceptable salt thereof, wherein Q is structure (a) in formula (I) above.

4. The alkyne derivative according to claim 2 above or a pharmaceutically acceptable salt thereof, wherein Q is structure (b) in formula (I) above.

5. The alkyne derivative according to claim 2 above or a pharmaceutically acceptable salt thereof, wherein Q is structure (c) in formula (I) above.

6. The alkyne derivative according to claim 2 above or a pharmaceutically acceptable salt thereof, wherein Q is structure (d) in formula (I) above.

7. The alkyne derivative according to claim 2 above or a pharmaceutically acceptable salt thereof, wherein Q is structure (m) in formula (I) above.

8. An alkyne derivative according to Examples 1 to 84 or a pharmaceutically acceptable salt thereof.

9. The alkyne derivative according to claim 1 above or a pharmaceutically acceptable salt thereof, which is selected from the group consisting of the following compounds:
(R)-1-([1,3]dioxolo[4′,5′:5,6]benzo[1,2-d]thiazol-7-yl)-5-(1-propyn-1-yl)imidazolidin-2-one (Example 2);
(4S,5R)-1-(7,8-dihydrobenzofuro[4,5-d]thiazol-2-yl)-4-methyl-5-(prop-1-yn-1-yl)imidazolidin-2-one (Example 5);
1-(7,8-dihydrobenzofuro[4,5-d]thiazol-2-yl)-5-(prop-1-yn-1-yl)imidazolidin-2-one (Example 9);
(4S,5R)-1-([1,3]dioxolo[4′,5′:5,6]benzo[1,2-d]thiazol-7-yl)-4-methyl-5-(prop-1-yn-1-yl)imidazolidin-2-one (Example 23);
(4S,5R)-1-(7,8-dihydro-[1,4]dioxyno[2′,3′:5,6]benzo[1,2-d]thiazol-2-yl)-4-methyl-5-(prop-1-yn-1-yl)imidazolidin-2-one (Example 56);
(4S,5R)-1-(8,9-dihydro-7H-chromeno[5,6-d]thiazol-2-yl)-4-methyl-5-(prop-1-yn-1-yl)imidazolidin-2-one (Example 57);
1-(7,8-dihydro-[1,4]dioxyno[2′,3′:5,6]benzo[1,2-d]thiazol-2-yl)-5-(prop-1-yn-1-yl)imidazolidin-2-one (Example 58);
(R)-1-([1,3]dioxolo[4′,5′:5,6]benzo[1,2-d]thiazol-7-yl-2,2-d2)-5-(prop-1-yn-1-yl)imidazolidin one (Example 62);
cis-1-(7,8-dihydro-[1,4]dioxyno[2′,3′:5,6]benzo[1,2-d]thiazol-2-yl)-4-(hydroxymethyl)-5-(prop-1-yn-1-yl)imidazolidin-2-one (Example 64);
cis-1-(7,8-dihydrobenzofuro[4,5-d]thiazol-2-yl)-4-ethyl-5-(prop-1-yn-1-yl)imidazolidin-2-one (Example 73);
cis-1-(7,8-dihydrobenzofuro[4,5-d]thiazol-2-yl)-4-(methoxymethyl)-5-(prop-1-yn-1-yl)imidazolidin-2-one (Example 74);
(4R,5R)-1-(7,8-dihydrobenzofuro[4,5-d]thiazol-2-yl)-4-((R)-1-hydroxyethyl)-5-(prop-1-yn-1-yl)imidazolidin-2-one (Example 82); and
(4R,5R)-1-(7,8-dihydrobenzofuro[4,5-d]thiazol-2-yl)-4-((S)-1-hydroxyethyl)-5-(prop-1-yn-1-yl)imidazolidin-2-one (Example 84).

10. A medicament comprising the alkyne derivative according to claim 1 or a pharmaceutically acceptable salt thereof as an active ingredient.

11. A pharmaceutical composition comprising the alkyne derivative according to claim 1 or a pharmaceutically acceptable salt thereof as an active ingredient.

12. A therapeutic agent and/or a prophylactic agent for a disease involving DYRK, comprising the alkyne derivative according to claim 1 or a pharmaceutically acceptable salt thereof as an active ingredient.

13. The therapeutic agent and/or the prophylactic agent according to claim 12 above, wherein the disease involving DYRK is frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and mental retardation associated therewith, memory impairment, memory loss, learning disability, intellectual disability, cognitive impairment, mild cognitive impairment, treatment for progression of dementia symptoms or prevention of dementia onset, or brain tumor, pancreatic cancer, ovarian cancer, osteosarcoma, colorectal cancer, lung cancer, bone resorption disease, osteoporosis, sickle cell anemia, or bone resorption disease in chronic kidney disease.

14. A method for treating and/or preventing a disease involving DYRK, comprising administering to a patient in need of the treatment a therapeutically effective amount of the alkyne derivative according to claim 1 or a pharmaceutically acceptable salt thereof.

15. Use of the alkyne derivative according to claim 1 or a pharmaceutically acceptable salt thereof, for producing a therapeutic agent and/or a prophylactic agent for a disease involving DYRK.

16. The alkyne derivative according to claim 1 or a pharmaceutically acceptable salt thereof, for use in the treatment and/or prevention of a disease involving DYRK.

17. A medicament comprising the medicament according to claim 10 above in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.

18. The medicament according to claim 17 above, for treating frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and a complication associated therewith, mental retardation, memory impairment, memory loss, learning disability, intellectual disability, cognitive impairment, mild cognitive impairment, or treatment for progression of dementia symptoms or prevention of dementia onset; or treating brain tumor, pancreatic cancer, ovarian cancer, osteosarcoma, colorectal cancer, lung cancer, bone resorption disease, osteoporosis, sickle cell anemia, or bone resorption disease in chronic kidney disease in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.
